(19) United States
(12) Patent Application Publication
(10) Pub. No.: US 2025/0388899 A1
(43) Pub. Date: Dec. 25, 2025

(54) RNA INTERFERENCE OLIGONUCLEOTIDES FOR INHIBITING PERINEURONAL NETWORK FORMATION

(71) Applicant: Nurexone Biologic Ltd., Haifa (IL)

(72) Inventors: Lyora AHARONOV, Haifa (IL); Nisim PERETS, Tel Aviv (IL); Lior SHALTIEL, Modi'in (IL)

(21) Appl. No.: 18/992,509

(22) PCT Filed: Jul. 9, 2023
(86) PCT No.: PCT/IL2023/050712

§ 371 Date: Jan. 8, 2025

Related U.S. Application Data
(60) Provisional application No. 63/359,842, filed on Jul. 10, 2022.

(51) Int. Cl. C12N 15/113; A61K 9/50; A61K 45/06; A61P 25/28

(52) U.S. Cl. CPC C12N 15/113; A61K 9/5068; A61K 45/06; A61P 25/28; C12N 2310/11; C12N 2310/14; C12N 2310/3515; C12N 2310/531

(57) ABSTRACT

The present invention discloses to RNA interference (RNAi) oligonucleotides such as siRNA and shRNA inhibiting the expression of proteins involved in the formation of a Perineuronal Network. Specifically provided RNAi oligonucleotides inhibiting expression of Neurocan or Tenascin-R. Also provided extracellular vesicles comprising said RNAi molecules and pharmaceutical compositions comprising said RNAi oligonucleotides or extracellular vesicles. The invention further discloses the use of such pharmaceutical compositions in the treatment of neural damage or injury.

### FIELD OF THE INVENTION

The present invention relates to RNA interference (RNAi) oligonucleotides inhibiting expression of proteins involved in the formation of a perineural network, to extracellular vesicles and, compositions comprising said RNAi oligonucleotides or extracellular vesicles and uses thereof.

### BACKGROUND OF THE INVENTION

Spinal cord injury is considered to be a chronic, irreversible condition. To date, there is no effective treatment that can lead to functional recovery after severe spinal cord injury. There are several neurological mechanisms underlying the neurons' limited ability to regenerate after transection. One of these mechanisms is the Perineuronal Network (PNN), an extracellular matrix that inhibits the ability of the neurons for plasticity in adulthood, thus also limiting their ability to regenerate.

The PNN is composed of several proteins of the lectican family of chondroitin sulfate proteoglycans (CsPGs): Neurocan (NCAN), Tenascin-R (TNR), Versican (VCAN) and Brevican (BCAN) and Aggrecan (ACAN), which are highly organized in a ternary stable structure. These proteins are being synthesized by the neurons to maintain the synaptic stabilization in the adult brain. PNNs are dynamic scaffolds that are involved in plasticity modulation. Pathological studies have shown that following spinal cord injuries (SCI) there is a noticeable increase in the expression of CsPGs at the lesion area which most likely hinders axon regeneration and plasticity following SCI.

The use of siRNA for protein inhibition in the Central Nervous System (CNS) requires a smart delivery method that can carry the siRNA into the target cells and keep its functionality. Exosomes are small lipid nano-vesicles that are naturally used as a cell-to-cell communication. Several studies demonstrated that exosomes may be loaded and used as a carrier for therapeutic agents.

Guo et al., (ACS Nano. 2019; 13 (9): 10015-10028. doi: 10.1021/acsnano.9b01892; Perets et al., Nano Lett. 2019; 19 (6): 3422-3431. doi: 10.1021/acs.nanolett.8b04148) have previously shown that exosomes derived from mesenchymal stem cells (MSC-exo) can specifically accumulate in inflammatory areas in the CNS after intranasal administration. Specifically, WO 2019186558 demonstrates that MSC-exo can be loaded with siRNA against phosphatase and tensin homolog (PTEN), for delivery and regeneration promotion of the spinal cord after complete transection. Since siRNA inhibits the expression of a specific protein within the cell, the delivery method needs to preserve this ability and enable the siRNA uptake by the target cell. Exosomes are an excellent delivery system in this aspect since they naturally undergo uptake by their target cells.

Several methods have been demonstrated to decompose the PNN in order to study its function. The most common strategy of PNN decomposition includes the use of enzymatic digestion of the PNN with chondroitinase ABC (O'Dell D E, Schreurs B G, Smith-Bell C. Wang D. Neurobiol Learn Mem. 2021; 177 (December 2020): 107358. doi: 10.1016/j.nlm.2020.107358). Chondroitinase ABC (ChABC) is an enzyme obtained from bacteria named Proteus vulgaris and it acts by degrading the glycosaminoglycan side chains of CSPGs. It has been shown that the use of ChABC can temporarily degrade the PNN thus allowing neuronal regeneration of the spinal cord post-injury. However, ChABC does not selectively degrade a specific PNN protein and may have a range influence on several other mechanism such immune regulation response by influencing IL-10. Currently, no efficient methods and moreover therapies for destabilization of PNN are currently available. Such methods may be an efficient treatment in case of spinal cord injury and therefore are highly needed.

### SUMMARY OF THE INVENTION

The present invention is based on the development of novel RNA interference (RNAi) oligonucleotide, such as siRNA molecules that are capable of inhibiting the expression of several proteins that play an important role in the formation of perineuronal nets (PNN). Specifically, inhibition of Neurocan (NCAN), Tenascin-R (TNR) or both is achieved.

As described earlier, the PNN was mainly observed in mature neurons and takes a part in plasticity restriction. Therefore, interfering with the formation of PNN allows greater neural plasticity and subsequently neural regeneration. Unlike enzymatic degradation, RNA-based inhibition of protein expression is more specific and controllable. Since PNN is a protein-based structure, its structure can be disrupted by inhibiting the expression of one of the proteins forming it. Extracellular Vesicles (EVs) derived from mesenchymal stem cells (MSC-exo) are used as a delivery system. These EVs previously demonstrated the natural ability to accumulate in the inflammatory areas and also convey natural therapeutic ability. Thus, additive and even synergistic effect between the short-term inhibition of the PNN formation and the MSC-exo was anticipated. The present invention relates to RNAi oligonucleotides capable of inhibiting expression of proteins that are part of the PNN matrix, specifically RNAi inhibiting expression of a protein selected from Neurocan (NCAN), Tenascin-R (TNR), Aggrecan (ACAN), Versican (VCAN) and Brevican (BCAN), to isolated EVs comprising said RNAi molecules or combination thereof, as well as to pharmaceutical composition comprising said EVs and their use in treating neurological conditions.

According to one aspect, the present invention provides an RNAi oligonucleotide, selected from siRNA and shRNA comprising a guide strand, inhibiting the expression of a protein of a perineuronal network. According to some examples, the protein of a perineuronal network selected from Neurocan (NCAN) and Tenascin-R (TNR). According to examples, the present invention provides an RNA interference (RNAi) oligonucleotide selected from siRNA and shRNA comprising a guide strand comprising a nucleic acid sequence selected from SEQ ID NO: 1-5 and 30-43. In some examples RNAi oligonucleotide comprising a guide strand comprising a nucleic acid sequence selected from SEQ ID NO: 1-5 is for inhibiting expression of Neurocan (NCAN). In some examples, RNAi oligonucleotide comprising a guide strand comprising a nucleic acid sequence selected from SEQ ID NO: 30-43 is for inhibiting expression of Tenascin-R (TNR). Thus, in some examples, the present invention provides an RNA interference (RNAi) oligonucleotide selected from siRNA and shRNA comprising a guide strand comprising a nucleic acid sequence selected from SEQ ID NO: 1-5 and 30-43 for inhibiting expression of a protein of a perineuronal network selected from Neurocan (NCAN) and Tenascin-R (TNR).

According to some examples, the present invention provides an RNA interference (RNAi) oligonucleotide comprising a guide strand comprising a nucleic acid sequence selected from SEQ ID NO: 1-5, 11-15 and 86-95, wherein the RNAi oligonucleotide inhibits the expression of Neurocan. According to some examples, the RNAi oligonucleotide is selected from siRNA and shRNA. According to some examples, the RNAi oligonucleotide is siRNA and the guide strand consists of a nucleic acid sequence selected from SEQ ID NO: 1-5, 11-15, and 86-95. According to some examples, the RNAi oligonucleotide comprises a strand complementary to said guide strand, wherein the complementary strand is complementary to at least 14, 15, 16, 17, 18, 19, 20 or 21 contiguous nucleotides of the guide strand. According to some examples, the complementary strand comprises from 14 to 19 nucleotides. According to some examples, the complementary strand comprises a nucleic acid sequence selected from SEQ ID NO: 6-10, 16-20 and 96-105.

According to other examples, the present invention provides an RNA interference (RNAi) oligonucleotide comprising a guide strand comprising a nucleic acid sequence selected from SEQ ID NO: 30-43 and 58-71, wherein the RNAi oligonucleotide inhibits the expression of Tenascin-R (TNR). According to some examples, the RNAi oligonucleotide is selected from siRNA and shRNA. According to some examples, the RNAi oligonucleotide is siRNA and wherein the guide strand consists of a nucleic acid sequence selected from SEQ ID NO: 58-71. According to some examples, the RNAi comprises a strand complementary to said guide strand, wherein the complementary strand is complementary to at least 14, 15, 16, 17, 18, 19, 20 or 21 contiguous nucleotides of the guide strand.

According to some examples, the complementary strand comprises from 14 to 19 nucleotides. According to some examples, the complementary strand comprises a nucleic acid sequence selected from SEQ ID NO: 44-57 and 72-85.

According to some examples, the present invention provides a conjugate of the RNAi as defined above with another moiety. According to some examples, the RNAi is conjugated with a hydrophobic molecule, e.g., selected from the group consisting of a sterol, a ganglioside, a lipid, a vitamin, a fatty acid, a hydrophobic peptide, and a combination thereof. According to some examples, the RNAi is conjugated with a hydrophilic moiety. In some examples, the hydrophilic moiety is a carbohydrate. In some examples, the carbohydrate is selected from glucose and sucrose.

According to another aspect, the present invention provides isolated extracellular vesicles (EVs) comprising RNA interference (RNAi) oligonucleotides inhibiting the expression of a protein of a perineuronal network. In some examples the RNAi oligonucleotides are selected from siRNA and shRNA. In some examples, the protein of a perineuronal network is selected from Neurocan (NCAN), Tenascin-R. Aggrecan (ACAN), Versican (VCAN), Brevican (BCAN) and a combination thereof. According to some examples, the EVs are selected from exosomes, microvesicles, and a combination thereof. According to some examples, the isolated EVs comprise RNAi oligonucleotides inhibiting the expression of NCAN as described in the application. According to some embodiments, the isolated EVs comprise RNAi oligonucleotides inhibiting the expression of Tenascin-R as described in the application.

According to another aspect, the present invention provides a pharmaceutical composition comprising the RNAi oligonucleotides or the isolated EVs as defined in the examples and embodiments of the present application, and a pharmaceutically acceptable carrier. According to some embodiments, the pharmaceutical composition is formulated for administration via an administration route selected from intranasal, intra-lesion, intrathecal, intravenous, intramuscular, subcutaneous, sublingual, oral, and intracerebral administration routes. According to some embodiments, the pharmaceutical composition is for use in treating a neuronal injury or damage in a subject. According to some embodiments, the pharmaceutical composition is for use in increasing neural plasticity and/or neural regeneration, optionally after neuronal injury or damage. According to one embodiment, the neuronal injury or damage is a spinal cord injury (SCI). According to some particular embodiments, the use comprises intranasal administration of the composition.

According to another aspect, the present invention provides a method of treating a neuronal injury or damage in a subject in need thereof, comprising administering to the subject a therapeutically effective amount of isolated extracellular vesicles comprising an inhibitor of the expression of a protein selected from Neurocan (NCAN), Tenascin-R (TNR), Aggrecan (ACAN), Versican (VCAN), Brevican (BCAN) and a combination thereof. According to some embodiments, the method comprises administering EVs comprising the RNAi oligonucleotides of the present invention inhibiting the expression of NCAN and/or the TNR. According to some embodiments, the method comprises intranasal administering of the EVs.

### BRIEF DESCRIPTION OF THE DRAWINGS

FIG. 1 shows the effect of different anti-TNR siRNAs (20 nM) on the expression of TNR gene in SK—N-SH cells transfected using Lipofectamine™ 3000 reagent. Relative expression was evaluated with TaqMan probes by qRT-PCR. Expression of GAPDH gene was used as an internal control.

FIG. 2 shows the effect of TNR1, TNR2, TNR7 and TNR8 siRNAs (20 nM) on the expression of TNR in SK—N-SH cells transfected using Lipofectamine™ 3000 reagent for 24 hours. Relative expression was evaluated with TaqMan probes by qRT-PCR. Expression of GAPDH gene was used as internal control.

FIG. 3 shows the effect of different anti-NCAN siRNAs (20 nM) on the expression of NCAN gene in SK—N-SH cells transfected using Lipofectamine™ 3000 reagent. NCAN1 siRNA is denoted in the Figure as duplex 1, NCAN2 as duplex 2, NCAN5=duplex5; NCAN6=duplex6, NCAN7=duplex7, and NCAN8=duplex8.

FIG. 4 shows the effect of a 48 hours transfection of SK—N-SH cells with 10 or 20 nm of NCAN5 or NCAN6 siRNAs on NCAN gene expression.

FIG. 5 shows the efficacy of NCAN5 and NCAN6 siRNAs on NCAN gene expression in comparison to commercially available anti-NCAN siRNAs IDT1, IDT2 and IDT3.

FIGS. 6A and 6B show NCAN gene (FIG. 6A) and protein (FIG. 6B) expression following a 48 hours transfection with 20 nm of SK—N-SH cells with NCAN5 and NCAN6 siRNAs. 48 hr post-transfection RNA was isolated and the media was collected. Protein levels were evaluated with NCAN ELISA kit. Relative expression was evaluated with TaqMan probes by qRT-PCR.

### DETAILED DESCRIPTION OF THE INVENTION

Unless otherwise defined, all technical and/or scientific terms used herein have the same meaning as commonly understood by one of ordinary skill in the art to which the invention pertains. In case of conflict, the patent specification, including definitions, will control.

The present invention provides RNA interference oligonucleotides such as siRNA or shRNA capable of inhibiting the expression of at least one protein forming a perineuronal network (PNN) and subsequently capable of inhibiting the formation of said PNN. Such oligonucleotides may be useful in allowing neuroregeneration after neuronal injury or damage, which is commonly inhibited or prevented by PNN formation.

### NCAN

In one aspect, the present invention provides RNA silencing oligonucleotides for inhibiting the expression of Neurocan. According to some embodiments, RNA silencing oligonucleotides are RNA interference (RNAi) oligonucleotides. According to some embodiments, the RNAi oligonucleotides, i.e. siRNA or shRNA are designed to bind to a sequence of NCAN mRNA within a region of bases numbers 1000 to 1300, preferably within a region of bases numbers 1100 to 1200 in the sequence SEQ ID NO: 21. According to some embodiments, the RNAi oligonucleotides, i.e. siRNA or shRNA are designed to bind to a sequence of NCAN mRNA within a region of bases numbers 3700 to 4000, preferably within a region of bases numbers 3800 to 3900 in the sequence SEQ ID NO: 21. According to some embodiments, the RNAi oligonucleotides, i.e. siRNA or shRNA are designed to bind to a sequence of NCAN mRNA within a region of bases numbers 500 to 800, preferably within a region of bases numbers 600 to 700 in the sequence SEQ ID NO: 21. According to some embodiments, the RNAi comprises a guide strand comprising a nucleic acid sequence selected from SEQ ID NO: 1-5. According to some embodiments, the RNAi is selected from siRNA and shRNA. Thus, according to some embodiments, the present invention provides an RNA interference (RNAi) oligonucleotide selected from siRNA and shRNA comprising a guide strand comprising a nucleic acid sequence selected from SEQ ID NO: 1-5. According to some embodiments, the RNAi oligonucleotide is for inhibiting the expression of Neurocan. The sequences of the present invention are summarized in Table 1. According to some embodiments, the RNAi, such as siRNA and shRNA, comprises a guide strand comprising a nucleic acid sequence selected from SEQ ID NO: 11-15.

The term “polynucleotide” as used herein refers to a long nucleic acid comprising more than 150 nucleotides. The term “oligonucleotide” as used herein refers to a short single-stranded or double-stranded sequence of nucleic acids such as ribonucleic acid (RNA), deoxyribonucleic acid (DNA) or mimetics thereof, said nucleic acid has typically less than or equal to 150 nucleotides. According to some embodiments, the oligonucleotide consists of 2 to 150, 10 to 100, or 14 to 50 nucleotides. According to other embodiments, the oligonucleotide consists of from 15 to 40, from 17 to 35, or from 18 to 30 nucleic acids.

As used herein, the terms “RNA silencing agent”, “RNA silencing molecule” and “RNA silencing oligonucleotide” are used herein interchangeably and refer to an RNA that is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism, e.g. by degradation of mRNA via RNA interference. RNA silencing agents include noncoding RNA molecules, for example, RNA duplexes comprising paired strands, as well as precursor RNAs from which such small non-coding RNAs can be generated. Exemplary RNA silencing agents, referred also as RNA interference oligonucleotides, include dsRNAs such as siRNAs, miRNAs, and shRNAs. In one embodiment, the RNA silencing agent is capable of inducing RNA interference. In another embodiment, the RNA silencing agent is capable of mediating translational repression.

The term “RNA interference” refers to the process of sequence-specific post-transcriptional gene silencing in animals mediated by RNA interference oligonucleotides such as short interfering RNAs (siRNAs) and shRNAs. The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

The term “small interfering RNA” and “siRNA” refer to small inhibitory RNA duplexes (generally between 18-30 base-pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC. Shorter siRNA such as including 19 to 20 nucleotides (nt) in the mRNA binding strand. Typically, artificial siRNA appears as a guide (antisense) strand oligonucleotide 21mer that interact with mRNA and a complementary shorter strand (sense, usually 19mer) complementary to said guide strand. As used herein, the term “complementary” refers to the ability of a first polynucleotide to hybridize to a second polynucleotide under certain conditions.

It has been found that position of the 3′-overhang influences the potency of a siRNA and asymmetric duplexes having a 3′-overhang on the antisense strand are generally more potent than those with the 3′-overhang on the sense strand. This can be attributed to asymmetrical strand loading into RISC, as the opposite efficacy patterns are observed when targeting the antisense transcript.

According to some embodiments, the RNAi is siRNA. According to some embodiments, the siRNA inhibiting expression of NCAN comprises a guide strand comprising a nucleic acid sequence selected from SEQ ID NO: 1-5. According to some embodiments, the siRNA comprises a guide strand consisting of a nucleic acid sequence selected from SEQ ID NO: 1-5. According to some embodiments, the siRNA inhibiting expression of NCAN comprises a guide strand comprising a nucleic acid sequence selected from SEQ ID NO: 11-15 and 86-95. According to some embodiments, the siRNA comprises a guide strand consisting of a nucleic acid sequence selected from SEQ ID NO: 11-15 and 86-95.

The terms “guide strand”, “antisense strand” and “guide strand oligonucleotide” are used herein interchangeably and refer to a strand of siRNA or shRNA that directs binding to mRNA molecule, and therefore, complementary to it.

As used herein the term “inhibiting expression of X” has the meaning of inhibiting the expression of the gene X and inhibiting the production of X protein.

According to other embodiments, the RNAi is shRNA. According to some embodiments, the shRNA inhibiting expression of NCAN comprises a nucleic acid sequence selected from SEQ ID NO: 1-5. According to some embodiments, the shRNA inhibiting expression of NCAN comprises a nucleic acid sequence selected from SEQ ID NO: 11-15 and 86-95.

The term “shRNA”, as used herein, refers to an RNA agent having a stem-loop structure, comprising a first and second region of the complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is as known in the art and may vary e.g. including 3 to 23, or 5 to 15, or 7 to 13, or 4 to 9, or 9 to 11 nt. Some of the nucleotides in the loop can be involved in base-pair interactions with other nucleotides in the loop. Typically the shRNA molecule has less than approximately 400 to 500 nucleotides (nt), or less than 100 to 200 nt, in which at least one stretch of at least 14 to 100 nucleotides (e.g., 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity.

According to some embodiments, the RNAi oligonucleotide, such as siRNA or shRNA, is not natural RNAi, i.e., does not exist in nature and is being artificially designed, chemically modified and manufactured. According to some embodiments, the siRNA is an artificial siRNA. According to some embodiments, the shRNA is an artificial shRNA.

The term “Neurocan” refers to human chondroitin sulfate proteoglycan protein having UniProtKB ID of 014594.

According to some embodiments, the RNAi oligonucleotide, e.g., siRNA or shRNA comprises a complementary strand, i.e., a strand complementary to said guide strand. According to some embodiments, the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises from 14 to 19 nucleotides. According to some embodiments, the complementary strand comprises 14, 15, 16, 17, 18 or 19 nucleotides. According to some embodiments, the complementary strand comprises a nucleic acid sequence selected from SEQ ID NO: 6-10, wherein the complementary strand comprises at positions 1 and 19 nucleic acids that are complementary to the nucleic acids at the corresponding positions in the sequence of the guide strand. According to some embodiments, the complementary strand comprises a nucleic acid sequence selected from SEQ ID NO: 16-20 and 96-105. As shown in Table 1, SEQ ID NOs: 1-10 comprise N that may be any nucleotide. Here and in any embodiments of the present application where applicable, N at position 1 of the guide strand is complementary to N at position 19 of the corresponding complementary (sense) strand and N at position 19 of the guide strand is complementary to N at position 1 of the corresponding sense strand, the position is counted from the 5′ to the sequence of the oligonucleotides.

According to some embodiments, the RNAi oligonucleotide inhibiting expression of NCAN is siRNA comprising a guide strand comprising or consisting of a nucleic acid sequence selected from SEQ ID NO: 1-5, 11-15 and 86-95. According to another embodiment, the siRNA inhibiting expression of NCAN comprises a complementary strand comprising a nucleic acid sequence selected from SEQ ID NO: 6-10 16-20 and 96-105, wherein the complementary strand comprises at positions 1 and 19 nucleic acids that are complementary to the nucleic acids at the corresponding positions in the sequence of the guide strand. According to some embodiments, the siRNA inhibiting expression of NCAN comprises a pair of oligonucleotides comprising or consisting of nucleic acid sequences (i) SEQ ID NO: 1 and 5; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the siRNA inhibiting expression of NCAN comprises a pair of oligonucleotides comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (xv) SEQ ID NOs: 95 and 105. According to some embodiments, the present invention provides a siRNA comprising a guide strand comprising the nucleic acid sequences SEQ ID NOs: 11 and a complementary strand comprising the nucleic acid sequences SEQ ID NO: 16. According to some embodiments, the present invention provides a siRNA comprising a guide strand comprising the nucleic acid sequences SEQ ID NOs: 12 and a complementary strand comprising the nucleic acid sequences SEQ ID NO: 17. According to some embodiments, the present invention provides a siRNA comprising a guide strand comprising the nucleic acid sequences SEQ ID NOs: 13 and a complementary strand comprising the nucleic acid sequences SEQ ID NO: 18.

According to some embodiments, the present invention provides a siRNA comprising a guide strand comprising the nucleic acid sequences SEQ ID NOs: 86 and a complementary strand comprising the nucleic acid sequences SEQ ID NO: 96. According to some embodiments, the present invention provides a siRNA comprising a guide strand comprising the nucleic acid sequences SEQ ID NOs: 87 and a complementary strand comprising the nucleic acid sequences SEQ ID NO: 97. According to some embodiments, the present invention provides a siRNA comprising a guide strand comprising the nucleic acid sequences SEQ ID NOs: 88 and a complementary strand comprising the nucleic acid sequences SEQ ID NO: 98. According to some embodiments, the present invention provides a siRNA comprising a guide strand comprising the nucleic acid sequences SEQ ID NOs: 89 and a complementary strand comprising the nucleic acid sequences SEQ ID NO: 99.

According to some embodiments, the present invention provides a siRNA comprising a guide strand comprising the nucleic acid sequences SEQ ID NOs: 90 and a complementary strand comprising the nucleic acid sequences SEQ ID NO: 100. According to some embodiments, the present invention provides a siRNA comprising a guide strand comprising the nucleic acid sequences SEQ ID NOs: 91 and a complementary strand comprising the nucleic acid sequences SEQ ID NO: 101. According to some embodiments, the present invention provides a siRNA comprising a guide strand comprising the nucleic acid sequences SEQ ID NOs: 92 and a complementary strand comprising the nucleic acid sequences SEQ ID NO: 102. According to some embodiments, the present invention provides a siRNA comprising a guide strand comprising the nucleic acid sequences SEQ ID NOs: 93 and a complementary strand comprising the nucleic acid sequences SEQ ID NO: 103. According to some embodiments, the present invention provides a siRNA comprising a guide strand comprising the nucleic acid sequences SEQ ID NOs: 94 and a complementary strand comprising the nucleic acid sequences SEQ ID NO: 104. According to some embodiments, the present invention provides a siRNA comprising a guide strand comprising the nucleic acid sequences SEQ ID NOs: 95 and a complementary strand comprising the nucleic acid sequences SEQ ID NO: 105. According to some embodiments, the RNAi oligonucleotide inhibiting expression of NCAN is shRNA comprising a guide strand comprising or consisting of a nucleic acid sequence selected from SEQ ID NO: 1-5, 11-15 and 86-95. According to another embodiment, the shRNA inhibiting expression of NCAN comprises a complementary strand comprising a nucleic acid sequence selected from SEQ ID NO: 6-10, and 96-105 wherein the complementary strand comprises nucleic acids at positions 1 and 19 that are complementary to the nucleic acids at the corresponding positions in the sequence of said guide strand. According to some embodiments, the shRNA inhibiting expression of NCAN comprises a pair of oligonucleotides comprising or consisting of nucleic acid sequences (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N at position 1 of the guide strand is complementary to N at position 19 of the corresponding sense strand and N at position 19 of the guide strand is complementary to N at position 1 of the corresponding sense strand, the position is counted from the 5′ to the sequence of the oligonucleotide. According to some embodiments, the shRNA inhibiting expression of NCAN comprises a pair of oligonucleotides comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (xv) SEQ ID NOs: 95 and 105. According to some embodiments, the present invention provides a shRNA comprising a guide strand comprising the nucleic acid sequences SEQ ID NOs: 11 and a complementary strand comprising the nucleic acid sequences SEQ ID NO: 16. According to some embodiments, the present invention provides a shRNA comprising a guide strand comprising the nucleic acid sequences SEQ ID NOs: 12 and a complementary strand comprising the nucleic acid sequences SEQ ID NO: 17. According to some embodiments, the present invention provides a shRNA comprising a guide strand comprising the nucleic acid sequences SEQ ID NOs: 13 and a complementary strand comprising the nucleic acid sequences SEQ ID NO: 18.

According to some embodiments, the RNAi oligonucleotide of the present invention is conjugated with another moiety. According to some embodiments, the RNAi oligonucleotide of the present invention is conjugated with a hydrophilic moiety. According to some embodiments, the RNAi oligonucleotide of the present invention is conjugated with a hydrophobic moiety. Thus, according to some embodiments, the siRNA or the shRNA oligonucleotide of the present invention is conjugated with a hydrophobic moiety. According to some embodiments, the hydrophobic molecule is bound to the guide strand. According to some embodiments, the hydrophobic molecule is bound to the complementary strand. According to some embodiments, the moiety is a loading moiety. The term “loading moiety” refers to a moiety allowing or enhancing the loading of molecules into EVs.

According to one embodiment, the said hydrophobic moiety is selected from the group consisting of a sterol, a ganglioside, a lipid, a vitamin, a fatty acid, a peptide, and a combination thereof. According to one embodiment, the RNA interference oligonucleotide is conjugated with a sterol. In exemplary embodiments, the moiety is a sterol cholesterol molecule, therefore according to such embodiments, the RNA interference oligonucleotide is conjugated with cholesterol. According to some embodiments, one of the strands of the double-stranded RNAi is conjugated with a hydrophobic molecule such as cholesterol.

According to other embodiments, two strands of the double-stranded RNAi are conjugated with a hydrophobic molecule such as cholesterol. According to other embodiments, the RNA interference oligonucleotide is conjugated with a molecule selected from monosialotetrahexosylganglioside (GM1), a lipid, a vitamin, a small molecule, a peptide, or a combination thereof. In some embodiments, the moiety is a lipid. For example, in certain embodiments, the moiety is palmitoyl. In some embodiments, the moiety is a sterol, e.g., cholesterol. Additional hydrophobic moieties include, for example, phospholipids, vitamin D, vitamin E, squalene, and fatty acids. In another exemplary embodiment, the RNAi oligonucleotide is conjugated to myristic acid, or a derivative thereof (e.g., myristoylated oligonucleotide cargo). In some embodiments, the hydrophobic moiety is conjugated at the termini of the oligonucleotide cargo (i.e., “terminal modification”). In other embodiments, the hydrophobic moiety is conjugated to other portions of the oligonucleotide molecule.

According to some embodiments, the RNAi oligonucleotide of the present invention is conjugated with a hydrophobic moiety selected from the group consisting of a sterol, a ganglioside, a lipid, a vitamin, a fatty acid, a hydrophobic peptide, and a combination thereof.

According to some embodiments, the siRNA is conjugated with cholesterol. According to some embodiments, the cholesterol is conjugated to the guide strand of siRNA. According to other embodiments, the cholesterol is conjugated to the complementary strand of siRNA. According to some embodiments, the cholesterol is conjugated to shRNA.

According to some embodiments, the present invention provides a siRNA comprising a guide strand comprising the nucleic acid sequences SEQ ID NOs: 11 and a complementary strand comprising the nucleic acid sequences SEQ ID NO: 16, wherein the siRNA is conjugated with cholesterol. According to some embodiments, the present invention provides a siRNA comprising a guide strand comprising the nucleic acid sequences SEQ ID NOs: 12 and a complementary strand comprising the nucleic acid sequences SEQ ID NO: 17, wherein the siRNA is conjugated with cholesterol. According to some embodiments, the present invention provides a siRNA comprising a guide strand comprising the nucleic acid sequences SEQ ID NOs: 13 and a complementary strand comprising the nucleic acid sequences SEQ ID NO: 18, wherein the siRNA is conjugated with cholesterol.

According to some embodiments, the siRNA or the shRNA oligonucleotide of the present invention for inhibiting the expression of Tenascin-R is conjugated with a hydrophilic moiety. According to some embodiments, the hydrophilic molecule is bound to the guide strand. According to some embodiments, the hydrophilic molecule is bound to the complementary strand. According to some embodiments, the hydrophilic moiety is a loading moiety. In some embodiments, the hydrophilic loading moiety is a carbohydrate or derivative thereof. In some embodiments, the hydrophilic loading moiety is a carbohydrate. According to some embodiments, the carbohydrate derivative is a conjugate of a carbohydrate with a lipid. According to some embodiments, the carbohydrate derivative comprises a carbohydrate linked with a lipid. According to some embodiments, the lipid is selected from phospholipids, fatty acids, triglycerides and amino alcohol such as serine and hydroxyproline. According to some embodiments, the carbohydrate is selected from a monosaccharide, disaccharide, trisaccharide, tetrasaccharide and oligosaccharide.

According to some embodiments, the carbohydrate is a monosaccharide. According to some embodiments, the monosaccharide is selected from glucose, fructose ribose, arabinose, galactose, mannose and xylose. According to some embodiments, the monosaccharide is glucose. According to some embodiments, the monosaccharide is fructose. According to some embodiments, the monosaccharide is arabinose. According to some embodiments, the carbohydrate is a disaccharide. According to some embodiments, the disaccharide is selected from sucrose, lactose and maltose. According to some embodiments, the disaccharide is sucrose. According to some embodiments, the carbohydrate is a trisaccharide. According to some embodiments, the trisaccharide is selected from maltotriose and raffinose. According to some embodiments, the carbohydrate is a tetrasaccharide. According to some embodiments, the carbohydrate is an oligosaccharide. According to some embodiments, the saccharide is selected from glucose, ribose, arabinose, galactose, mannose, sucrose and maltotriose. According to some embodiments, the hydrophilic loading moiety is glucose.

According to some embodiments, the siRNA is conjugated with glucose. According to some embodiments, the glucose is conjugated to the guide strand of siRNA. According to other embodiments, the glucose is conjugated to the complementary strand of siRNA. According to some embodiments, the glucose is conjugated to shRNA.

According to some embodiments, the loading moiety is bound to the siRNA or shRNA via a linker. According to some embodiments, the linker is selected from hydrophilic, hydrophobic, and amphiphilic linkers. According to some embodiments, the linker is a DBCO-C6-azide.

According to some embodiments, the present invention provides a siRNA comprising a guide strand and a complementary strand comprising the nucleic acid sequences SEQ ID NOs: 11 and SEQ ID NO: 16, respectively wherein the siRNA is conjugated with glucose, optionally via a linker. According to some embodiments, the present invention provides a siRNA comprising a guide strand and a complementary strand comprising the nucleic acid sequences SEQ ID NOs: 12 and SEQ ID NO: 17, respectively wherein the siRNA is conjugated with glucose, optionally via a linker. According to some embodiments, the present invention provides a siRNA comprising a guide strand and a complementary strand comprising the nucleic acid sequences SEQ ID NOs: 13 and SEQ ID NO: 18, respectively wherein the siRNA is conjugated with glucose, optionally via a linker.

The siRNA and shRNA molecules promote sequence-specific degradation of mRNA by RNAi to achieve inhibition of the expression of NCAN protein, or reduction of the expression level of the NCAN gene, e.g., by 95%, 90%, 85%, 80%, 75%, 70%, 60%, 50%, 40%, 30%, 20%, 10%, or 5%.

According to another aspect, the present invention provides a composition comprising the RNAi molecules of the present invention and a carrier. Any one of the above definitions, terms and embodiments are encompassed and apply herein as well. The term “carrier” as used herein refers to as a class any compound or composition useful in facilitating storage, stability, administration, cell targeting and/or delivery of the topical composition, including, without limitation, suitable vehicles, skin conditioning agents, skin protectants, diluents, emollients, solvents, excipients, pH modifiers, salts, colorants, rheology modifiers, thickeners, lubricants, humectants, antifoaming agents, crodible polymers, hydrogels, surfactants, emulsifiers, emulsion stabilizers, adjuvants, surfactants, preservatives, chelating agents, fatty acids, mono-di- and tri-glycerides and derivates thereof, waxes, oils and water. According to some embodiments, the present invention provides a composition comprising siRNA molecules comprising a pair of a guide strand and a complementary strand, wherein the pair comprises or consists of nucleic acid sequences SEQ ID NOs: 11 and 16; SEQ ID NOs: 12 and 17; or SEQ ID NO: 13 and 18, optionally wherein the siRNA is conjugated with cholesterol or glucose. According to some embodiments, the present invention provides a composition comprising siRNA molecules comprising a pair of a guide strand and a complementary strand, wherein the pair comprises or consists of nucleic acid sequences SEQ ID NOs: 86 and 96; SEQ ID NOs: 87 and 77; SEQ ID NO: 88 and 88; SEQ ID NOs: 89 and 99; SEQ ID NOs: 90 and 100; SEQ ID NOs: 91 and 101; SEQ ID NOs: 92 and 102; SEQ ID NOs: 93 and 103; SEQ ID NOs: 94 and 104; or SEQ ID NOs: 95 and 105, optionally wherein the siRNA is conjugated with cholesterol or glucose. In some embodiments, the carrier is a pharmaceutically acceptable carrier and the composition is a pharmaceutical composition.

### TNR

In one aspect, the present invention provides an RNA silencing oligonucleotide for inhibiting the expression of Tenascin-R (TNR). According to some embodiments, the RNA silencing oligonucleotides are RNA interference oligonucleotides. According to some embodiments, the RNAi oligonucleotides, i.e. siRNA or shRNA are designed to bind to a sequence of TNR mRNA within a region of bases numbers 1600 to 2000, preferably within a region of bases numbers 1700 to 1850 in the sequence SEQ ID NO: 22. According to some embodiments, the RNAi oligonucleotides, i.e. siRNA or shRNA are designed to bind to a sequence of TNR mRNA within a region of bases numbers 4300 to 4700, preferably within a region of bases numbers 4400 to 4500 in the sequence SEQ ID NO: 22. According to some embodiments, the RNAi inhibiting the expression of TNR comprises a guide strand comprising a nucleic acid sequence selected from SEQ ID NO: 30-43. According to some embodiments, the RNAi inhibiting the expression of TNR comprises a guide strand comprising a nucleic acid sequence selected from SEQ ID NO: 58-71. According to some embodiments, the RNAi is selected from siRNA and shRNA. Thus, according to some embodiments, the present invention provides an RNAi oligonucleotide selected from siRNA and shRNA comprising a guide strand comprising a nucleic acid sequence selected from SEQ ID NO: 30-43 and 58-71. According to some embodiments, the RNAi oligonucleotide is for inhibiting the expression of TNR.

According to some embodiments, the oligonucleotide consists of 2 to 150, 10 to 100, or 14 to 50 nucleotides. According to other embodiments, the oligonucleotide consists of from 15 to 40, from 17 to 35, or from 18 to 30 nucleic acids.

According to some embodiments, RNAi is selected from siRNAs, miRNAs and shRNAs.

According to some embodiments, the RNAi oligonucleotide is siRNA. According to some embodiments, the siRNA inhibiting the expression of TNR comprises a guide strand comprising a nucleic acid sequence selected from SEQ ID NO: 30-43. According to some embodiments, the siRNA comprises a guide strand consisting of a nucleic acid sequence selected from SEQ ID NO: 30-43. According to some embodiments, the siRNA inhibiting the expression of TNR comprises a guide strand comprising a nucleic acid sequence selected from SEQ ID NO: 58-71. According to some embodiments, the siRNA comprises a guide strand consisting of a nucleic acid sequence selected from SEQ ID NO: 58-71.

According to other embodiments, the RNAi is shRNA. According to some embodiments, the shRNA inhibiting the expression of TNR comprises a nucleic acid sequence selected from SEQ ID NO: 30-43 and 58-71.

The term “Tenascin-R” refers to a human extracellular matrix glycoprotein belonging to a family of Tenascins and having UniProtKB of Q92752.

According to some embodiments, the RNAi oligonucleotide, e.g., siRNA or shRNA comprises a complementary strand, i.e., a strand complementary to said guide strand. According to some embodiments, wherein the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises from 14 to 19 nucleotides. According to some embodiments, the complementary strand comprises 14, 15, 16, 17, 18 or 19 nucleotides.

According to some embodiments, the complementary strand comprises a nucleic acid sequence selected from SEQ ID NO: 44-57 wherein the complementary strand comprises nucleic acids at positions 1 and 19 that are complementary to the nucleic acids at the corresponding positions in the sequence of said guide strand. As shown in Table 1, SEQ ID NOs: 30-57 comprise N in which N may be any nucleotide, wherein N at position 1 of the guide strand is complementary to N at position 19 of the corresponding sense strand, and N at position 19 of the guide strand is complementary to N at position 1 of the corresponding sense strand, the position is counted from the 5′ to the sequence. In the sequence listing file, T represents U in the sequences of RNA molecules. According to some embodiments, the complementary strand comprises a nucleic acid sequence selected from SEQ ID NO: 72-85.

According to some embodiments, the RNAi oligonucleotide is siRNA inhibiting the expression of TNR comprising a guide strand comprising or consisting of a nucleic acid sequence selected from SEQ ID NO: 30-43 and 58-71. According to another embodiment, the siRNA comprises a complementary strand comprising a nucleic acid sequence selected from SEQ ID NO: 44-57 and 72-85 wherein the complementary strand comprises nucleic acids at positions 1 and 19 that are complementary to the nucleic acids at the corresponding positions in the sequence of said guide strand. According to some embodiments, the present invention provides siRNA comprising a pair of oligonucleotides comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the siRNA comprises a pair of oligonucleotides comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (v) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides a siRNA comprising a guide strand comprising the nucleic acid sequences SEQ ID NOs: 58 and a complementary strand comprising the nucleic acid sequences SEQ ID NO: 72. According to some embodiments, the present invention provides a siRNA comprising a guide strand comprising the nucleic acid sequences SEQ ID NOs: 61 and a complementary strand comprising the nucleic acid sequences SEQ ID NO: 75. According to some embodiments, the present invention provides a siRNA comprising a guide strand comprising the nucleic acid sequences SEQ ID NOs: 62 and a complementary strand comprising the nucleic acid sequences SEQ ID NO: 76.

According to some embodiments, the RNAi oligonucleotide is shRNA for inhibiting the expression of Tenascin-R comprises a guide strand comprising or consisting of a nucleic acid sequence selected from SEQ ID NO: 30-43 and 58-71. According to another embodiment, the shRNA comprises a complementary strand comprising a nucleic acid sequence selected from SEQ ID NO: 44-57 and 82-85. According to some embodiments, the shRNA comprises a pair of oligonucleotides comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the shRNA comprises a pair of oligonucleotides comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (vi) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides a shRNA comprising a guide strand comprising the nucleic acid sequences SEQ ID NOs: 58 and a complementary strand comprising the nucleic acid sequences SEQ ID NO: 72. According to some embodiments, the present invention provides a shRNA comprising a guide strand comprising the nucleic acid sequences SEQ ID NOs: 61 and a complementary strand comprising the nucleic acid sequences SEQ ID NO: 75. According to some embodiments, the present invention provides a shRNA comprising a guide strand comprising the nucleic acid sequences SEQ ID NOs: 62 and a complementary strand comprising the nucleic acid sequences SEQ ID NO: 76.

According to some embodiments, the RNAi oligonucleotide for inhibiting the expression of Tenascin-R of the present invention is conjugated with another moiety. According to some embodiments, the RNAi oligonucleotide of the present invention is conjugated with a hydrophobic moiety. According to some embodiments, the RNAi oligonucleotide of the present invention is conjugated with a hydrophobic molecule/moiety. According to some embodiments, the siRNA or the shRNA oligonucleotide of the present invention is conjugated with a hydrophobic molecule. According to some embodiments, the hydrophobic molecule is bound to the guide strand. According to some embodiments, the hydrophobic molecule is bound to the complementary strand.

According to one embodiment, the said hydrophobic moiety is selected from the group consisting of a sterol, a ganglioside, a lipid, a vitamin, a fatty acid, a peptide, and a combination thereof. According to one embodiment, the RNA interference oligonucleotide is conjugated with a sterol. In exemplary embodiments, the moiety is a sterol cholesterol molecule, therefore according to such embodiments, the RNA interference oligonucleotide is conjugated with a cholesterol. According to some embodiments, one of the strands of the double-stranded RNAi is conjugated with a hydrophobic molecule such as cholesterol. According to other embodiments, two strands of the double-stranded RNAi are conjugated with a hydrophobic molecule such as cholesterol. According to other embodiments, the RNA interference oligonucleotide is conjugated with a molecule selected from monosialotetrahexosylganglioside (GM1), a lipid, a vitamin, a small molecule, a peptide, or a combination thereof. In some embodiments, the moiety is a lipid. For example, in certain embodiments, the moiety is palmitoyl. In some embodiments, the moiety is a sterol, e.g., cholesterol. Additional hydrophobic moieties include, for example, phospholipids, vitamin D, vitamin E, squalene, and fatty acids. In another exemplary embodiment, the RNAi oligonucleotide is conjugated to myristic acid, or a derivative thereof (e.g., myristoylated oligonucleotide cargo). In some embodiments, the hydrophobic moiety is conjugated at the termini of the oligonucleotide cargo (i.e., “terminal modification”). In other embodiments, the hydrophobic moiety is conjugated to other portions of the oligonucleotide molecule.

According to some embodiments, the RNAi oligonucleotide of the present invention is conjugated with a hydrophobic moiety selected from the group consisting of a sterol, a ganglioside, a lipid, a vitamin, a fatty acid, a hydrophobic peptide, and a combination thereof.

According to some embodiments, the siRNA for inhibiting the expression of Tenascin-R is conjugated with cholesterol. According to some embodiments, the cholesterol is conjugated to the guide strand of siRNA. According to other embodiments, the cholesterol is conjugated to the complementary strand of siRNA. According to some embodiments, the cholesterol is conjugated to shRNA. According to some embodiments, the present invention provides a siRNA comprising a guide strand comprising the nucleic acid sequences SEQ ID NOs: 58 and a complementary strand comprising the nucleic acid sequences SEQ ID NO: 72, wherein the siRNA is conjugated with cholesterol. According to some embodiments, the present invention provides a siRNA comprising a guide strand comprising the nucleic acid sequences SEQ ID NOs: 61 and a complementary strand comprising the nucleic acid sequences SEQ ID NO: 75, wherein the siRNA is conjugated with cholesterol. According to some embodiments, the present invention provides a siRNA comprising a guide strand comprising the nucleic acid sequences SEQ ID NOs: 62 and a complementary strand comprising the nucleic acid sequences SEQ ID NO: 76, wherein the siRNA is conjugated with cholesterol.

According to some embodiments, the siRNA or the shRNA oligonucleotide of the present invention for inhibiting the expression of Tenascin-R is conjugated with a hydrophilic moiety. According to some embodiments, the hydrophilic molecule is bound to the guide strand. According to some embodiments, the hydrophilic molecule is bound to the complementary strand. According to some embodiments, the hydrophilic moiety is a loading moiety. In some embodiments, the hydrophilic loading moiety is a carbohydrate or derivative thereof. In some embodiments, the hydrophilic loading moiety is a carbohydrate. According to some embodiments, the carbohydrate is selected from a monosaccharide, disaccharide, trisaccharide, tetrasaccharide and oligosaccharide. According to some embodiments, the carbohydrate derivative is a conjugate of a carbohydrate with a lipid. According to some embodiments, the carbohydrate derivative comprises a carbohydrate linked with a lipid. According to some embodiments, the lipid is selected from phospholipids, fatty acids, triglycerides and amino alcohol such as serine and hydroxyproline. According to some embodiments, the carbohydrate is a monosaccharide. According to some embodiments, the monosaccharide is selected from glucose, fructose ribose, arabinose, galactose, mannose and xylose. According to some embodiments, the monosaccharide is glucose. According to some embodiments, the monosaccharide is fructose. According to some embodiments, the monosaccharide is arabinose. According to some embodiments, the carbohydrate is a disaccharide. According to some embodiments, the disaccharide is selected from sucrose, lactose and maltose. According to some embodiments, the disaccharide is sucrose. According to some embodiments, the carbohydrate is a trisaccharide. According to some embodiments, the trisaccharide is selected from maltotriose and raffinose. According to some embodiments, the carbohydrate is a tetrasaccharide. According to some embodiments, the carbohydrate is an oligosaccharide. According to some embodiments, the saccharide is selected from glucose, ribose, arabinose, galactose, mannose, sucrose and maltotriose. According to some embodiments, the hydrophilic loading moiety is glucose.

According to some embodiments, the siRNA is conjugated with glucose. According to some embodiments, the glucose is conjugated to the guide strand of siRNA. According to other embodiments, the glucose is conjugated to the complementary strand of siRNA. According to some embodiments, the glucose is conjugated to shRNA.

According to some embodiments, the loading moiety is bound to the siRNA or shRNA via a linker. According to some embodiments, the linker is selected from hydrophilic, hydrophobic, and amphiphilic linkers. According to some embodiments, the linker is a DBCO-C6-azide.

According to some embodiments, the present invention provides a siRNA comprising a guide strand and a complementary strand comprising the nucleic acid sequences SEQ ID NOs: 58 and SEQ ID NO: 72, respectively wherein the siRNA is conjugated with glucose, optionally via a linker. According to some embodiments, the present invention provides a siRNA comprising a guide strand and a complementary strand comprising the nucleic acid sequences SEQ ID NOs: 61 and SEQ ID NO: 75, respectively wherein the siRNA is conjugated with glucose, optionally via a linker. According to some embodiments, the present invention provides a siRNA comprising a guide strand and a complementary strand comprising the nucleic acid sequences SEQ ID NOs: 62 and SEQ ID NO: 76, respectively wherein the siRNA is conjugated with glucose, optionally via a linker.

According to another aspect, the present invention provides a composition comprising the RNAi oligonucleotide inhibiting TNR expression of the present invention and a carrier. Any one of the above definitions, terms and embodiments are encompassed and apply herein as well. The term “carrier” as used herein refers to as a class any compound or composition useful in facilitating storage, stability, administration, cell targeting and/or delivery of the topical composition, including, without limitation, suitable vehicles, skin conditioning agents, skin protectants, diluents, emollients, solvents, excipients, pH modifiers, salts, colorants, rheology modifiers, thickeners, lubricants, humectants, antifoaming agents, erodeable polymers, hydrogels, surfactants, emulsifiers, emulsion stabilizers, adjuvants, surfactants, preservatives, chelating agents, fatty acids, mono-di- and tri-glycerides and derivates thereof, waxes, oils and water.

According to some embodiments, the present invention provides a composition comprising siRNA molecules comprising a pair of a guide strand and a complementary strand, wherein the pair comprises or consists of nucleic acid sequences SEQ ID NOs: 58 and 72; SEQ ID NOs: 61 and 75; or SEQ ID NO: 62 and 76, optionally wherein the siRNA is conjugated with cholesterol or glucose. In some embodiments, the carrier is a pharmaceutically acceptable carrier and the composition is a pharmaceutical composition.

The siRNA and shRNA molecules promote sequence-specific degradation of mRNA by RNAi to achieve inhibition of the expression of the TNR protein or reduction of the expression level of the TNR gene, e.g., by 95%, 90%, 85%, 80%, 75%, 70%, 60%, 50%, 40%, 30%, 20%, 10%, or 5%.

### EVs

According to another aspect, the present invention provides isolated EVs comprising RNA interference (RNAi) oligonucleotides inhibiting the expression of a protein selected from Neurocan (NCAN), Tenascin-R (TNR) and a combination thereof, the present invention provides isolated EVs loaded with RNA interference (RNAi) oligonucleotides inhibiting the expression of a protein selected from Neurocan (NCAN), Tenascin-R (TNR) and a combination thereof. Any one of the above definitions, terms and embodiments are encompassed and apply herein as well. With respect to EVs, the term “loaded with” and “comprising” when referring to RNAi oligonucleotides may be used interchangeably.

According to some embodiments, the present invention provides isolated EVs comprising RNAi oligonucleotides inhibiting the expression of Neurocan. According to some embodiments, the present invention provides isolated EVs comprising RNAi oligonucleotides inhibiting the expression of Tenascin-R. According to some embodiments, the present invention provides isolated EVs comprising RNAi oligonucleotides inhibiting the expression of Neurocan and Tenascin-R.

According to some embodiments, the RNAi oligonucleotides are exogenous. The term “exogenous” as used herein refers to a molecule or substance (e.g., a compound, nucleic acid or protein) that originates from outside of a given membrane vesicle such as EVs, and is not naturally present in the vesicle. With respect to EVs, the term refers to molecules or substances that are not naturally present in the vesicle and not in the cells from which the EVs are derived. According to some embodiments, the term “exogenous” refers to synthetic (artificially synthesized) non-natural molecules. According to some embodiments, the substance is artificially loaded to the EVs or to cells from which the EVs are derived. With respect to peptides, proteins and nucleic acids the term means that the compound is artificially loaded to the EVs or to cells from which the vesicles are derived or artificially expressed within cells from which the vesicles are derived, however, the compound is not naturally expressed in the parent cells.

The terms “extracellular vesicles” and “EVs” are used herein interchangeably and refer to cell-derived vesicles comprising a membrane that encloses an internal space. Generally, EVs range in diameter from 30 nm to 1500 nm, more frequently from 40 to 1200 nm, and may comprise various cargo molecules either within the internal space, displayed on the external surface of the extracellular vesicle, and/or spanning the membrane. Said cargo molecules may comprise nucleic acids, proteins, carbohydrates, lipids, small molecules, and/or combinations thereof. EVs can be divided into three subpopulations: (I) exosomes: having a diameter of 30-150 nm in diameter and derived from endosomal compartments; (II) microvesicles: having a diameter of 100 nm-1 μm which are released from the cell surface via “vesiculation”; and (III) apoptotic bodies: having a diameter of 1-5 μm and which are released from apoptotic cells. The term EVs comprises also the terms “exosome” and “microvesicles”. The terms “exosomes” and “nanovesicle” are used herein interchangeably and refer to EVs having a size of between 30 to 150 nm in diameter. In some references, exosomes refer to EVs having a size of between 30 to 100 nm in diameter. The term “microvesicles” as used herein refers to EVs having a size of between 150 to 1000 nm in diameter. Generally, the EVs may comprise at least a part of the molecular contents of the cells from which they originated, e.g. lipids, fatty acids, polypeptides, polynucleotides, proteins and/or saccharides.

The EVs of the present invention are mostly spherical and the terms “size”, “particle size”, “average particle size” and “particle diameter size” used herein interchangeably refer to the diameter of the EVs or to the longest dimension of the EVs. Any known method for measurement of particle size may be used to determine the size of the EVs of the present invention. A non-limiting example is nanoparticle-tracking analysis (NTA).

According to some embodiments, the isolated EVs are exosomes. According to one embodiment, the exosomes have a diameter of from 30 to 150 nm, from 40 to 120 nm, from 50 to 100 nm, from 30 to 100 nm, from 30 nm to 80 nm, or from 60 nm to 80 nm.

According to another embodiment, the EVs are microvesicles. According to one embodiment, the microvesicles have a diameter of from 100 to 1000 nm, from 120 to 800 nm, from 150 to 600 nm or from 200 to 400 nm. According to another embodiment, the microvesicles have a size of 100 to 300 nm or 150 to 250 nm.

According to some embodiments, the EVs have a diameter of from 30 to 250 nm or from 50 to 200 nm. According to some embodiments, the EVs have a diameter of from 70 to 170 nm or from 80 to 150 nm.

The EVs may have a range of sizes, such as between 2 nm to 20 nm, 2 nm to 50 nm, 2 nm to 100 nm, 2 nm to 150 nm or 2 nm to 200 nm. The EVs may have a size between 20 nm to 50 nm, 20 nm to 100 nm, 20 nm to 150 nm or 20 nm to 200 nm. The EVs may have a size between 50 nm to 100 nm, 50 nm to 150 nm or 50 nm to 200 nm. The EVs may have a size between 100 nm to 150 nm or 100 nm to 200 nm. The EVs may have a size between 150 nm to 200 nm. The EVs may have a size of 100 to 600 nm, 150 to 500 nm, or 200 to 400 nm.

The size may be determined by various means. In principle, the size may be determined by size fractionation and filtration through a membrane with the relevant size cut-off.

According to a further embodiment, the isolated EVs are a combination of small and large vesicles, e.g., of microvesicles and exosomes.

As described hereinabove, the EVs are derived from cells. The terms “derived from” and “originated from” are used herein interchangeably and refer to vesicles that are produced within, by, or from, a specified cell, cell type, or population of cells. As used herein, the terms “parent cell”, “producer cell” and “original cell” include any cell from which the extracellular vesicle is derived and isolated. The term also encompasses a cell that shares a protein, lipid, sugar, or nucleic acid component of the extracellular vesicle. For example, a “parent cell” or “producer cell” includes a cell that serves as a source for the extracellular vesicle. According to some embodiments, the cells are eukaryotic cells.

The extracellular vesicles (EVs) may be derived from biological cells by any of several means, for example by secretion, budding or dispersal from the biological cells. The EVs may be something that is isolatable from a mesenchymal stem cell (MSC), neural crest cell (NCC), mesenchymal stem cell conditioned medium (MSC-CM) or neural crest cell conditioned medium. The EVs may be responsible for or have at least an activity of the parent cells such as of MSC, NCC, NCC-CM or MSC-CM. The EVs may be responsible for, and carry out, substantially most or all of the functions of the activity of the parent cells such as of MSC, NCC. NCC-CM or MSC-CM. For example, the EVs may be a substitute (or biological substitute) for the MSC, NCC, NCC-CM or MSC-CM. For example, the EVs may be produced, exuded, emitted or shed from the biological cells. Where the biological cell is in cell culture, the particle may be secreted into the cell culture medium.

Examples of biological cells from which the EVs may be derived include adherent cells which express mesenchymal markers such as mesenchymal stem cells, oral mucosa stem cells or olfactory ensheathing cells, astrocytes, and neural crest cells. Thus, according to some embodiments, the present invention provides a pharmaceutical composition comprising EVs loaded with an RNAi of the present invention, wherein the EVs are derived from adherent cells expressing mesenchymal markers. According to one embodiment, the adherent cells expressing mesenchymal markers are selected from mesenchymal stem cells (MSC), oral mucosa stem cells and olfactory ensheathing cells. According to one embodiment, the cells are mesenchymal stem cells (MSC). According to one embodiment, the EVs are derived from mesenchymal stem cells (MSC).

The term “mesenchymal stem cells” refers to multipotent stromal cells that can differentiate into a variety of cell types, as well known in the art, including to: osteoblasts (bone cells), chondrocytes (cartilage cells), myocytes (muscle cells) and adipocytes (fat cells).

In their pluripotent state, mesenchymal stem cells typically express the following markers: CD105, CD166, CD29, CD90, and CD73, and do not express CD34, CD45 and CD133.

Mesenchymal stem cells may be isolated from a variety of tissues including but not limited to bone marrow, adipose tissue, dental pulp, oral mucosa, peripheral blood and amniotic fluid. According to one embodiment, the mesenchymal stem cells are isolated from to bone marrow. According to one embodiment, the mesenchymal stem cells are originated from a site selected from bone marrow, adipose tissue, umbilical cord, dental pulp, oral mucosa, peripheral blood and amniotic fluid. According to some embodiments, the EVs are derived from bone marrow originated MSC. According to other embodiments, the EVs are derived from the adipose tissue originated MSC. According to some such embodiments, the EVs are selected from exosomes, microvesicles and a combination thereof. According to some embodiments, the cells express CD105, CD166, CD29, CD90, and CD73 markers. According to a further embodiment, the cells express CD105, CD166, CD29, CD90, and CD73, and do not express CD34, CD45 and CD133. According to some embodiments, the cells are selected from dental pulp stem cells (DPSCs), exfoliated deciduous teeth stem cells (SHED), periodontal ligament stem cells (PDLSCs), apical papilla stem cells (SCAP) and dental follicle progenitor cells (DFPCs).

According to some such embodiments, the EVs comprise or express at least a fraction of the markers expressed by the cell from which EVs are derived.

The EVs may comprise one or more proteins, oligonucleotides or polynucleotides secreted by a particular cell type e.g. mesenchymal stem cell or neural crest cell. The EVs may comprise one or more proteins or polynucleotides present in mesenchymal stem cell conditioned medium (MSC-CM). In a particular embodiment, the EVs may comprise miRNAs which are derived from MSCs or neural crest cells. For example, the EVs may comprise 10% or more, 20% or more, 30% or more, 40% or more, 50% or more, 60% or more or 70% or more of these proteins and/or polynucleotides. The EVs may comprise substantially about 75% of these proteins and/or polynucleotides. The proteins may be defined by reference to a list of proteins or gene products of a list of genes.

The EVs may have at least one property of a mesenchymal stem cell. The particle may have a biological property, such as biological activity. The particle may have any of the biological activities of an MSC. The particle may for example have a therapeutic or restorative activity of an MSC.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46 (12): 3349-60.

Mesenchymal stem cell cultures may be generated by diluting BM aspirates (usually 20 ml) with equal volumes of Hank's balanced salt solution (HBSS; GIBCO Laboratories, Grand Island, NY, USA) and layering the diluted cells over about 10 ml of a Ficoll column (Ficoll-Paque; Pharmacia, Piscataway, NJ, USA). Following 30 minutes of centrifugation at 2,500×g. the mononuclear cell layer is removed from the interface and suspended in HBSS. Cells are then centrifuged at 1,500×g for 15 minutes and resuspended in a complete medium (MEM, α medium without deoxyribonucleotides or ribonucleotides; GIBCO); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, NY) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, PA). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the mesenchymal stem cell fraction, frozen cells are thawed at 37° C. diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold (Colter D C., et al., Proc Natl Acad Sci USA. 97:3213-3218, 2000).

MSC cultures utilized by some embodiments of the invention include three groups of cells which are defined by their morphological features: small and agranular cells (referred to as RS-1, hereinbelow), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

According to a particular embodiment, the EVs are derived from cells expressing markers from neural crest cells. According to a particular embodiment, the EVs are derived from neural crest cells. According to another embodiment, the neural crest cells are cranial neural crest cells. According to some embodiments, the cranial neural crest cells include, but are not limited to dental pulp stem cells (DPSCs), exfoliated deciduous teeth stem cells (SHED), periodontal ligament stem cells (PDLSCs), apical papilla stem cells (SCAP) and dental follicle progenitor cells (DFPCs). According to some embodiments, such cells express mesenchymal markers, as defined above.

The EVs may be produced or isolated in a number of ways. Such a method may comprise isolating the EVs from mesenchymal stem cells (MSC) or from neural crest cells (NCC).

Therefore, the EVs of the present invention are isolated EVs.

The terms “purify.” “purified,” “purifying”, “isolate”, “isolated,” and “isolating” are used herein interchangeably and refer to the state of a population (e.g., a plurality of known or unknown amount and/or concentration) of EVs, that have undergone one or more processes of purification/isolation, e.g., a selection of the desired EVs, or alternatively a removal or reduction of residual biological products and/or removal of undesirable EVs, e.g. removing EVs having a particular size. According to one embodiment, the ratio of EVs to residual parent cells is at least 2, 3, 4, 5, 6, 8 or 10 times higher, or in certain advantageous embodiments at least 50, 100, 1000, or 2000 times higher than in the initial material. According to some embodiments, the ratio is the weight ratio. In some advantageous embodiments, the term “isolated” has the meaning of substantially cell-free or cell-free and may be substituted by it.

According to some embodiments, the EVs, e.g., exosomes are derived from adherent cells expressing mesenchymal markers. According to some embodiments, the adherent cells expressing mesenchymal markers are selected from mesenchymal stem cells (MSC) and olfactory ensheathing cells.

According to some embodiments, the present invention provides isolated EVs loaded with RNAi oligonucleotides inhibiting the expression of a protein NCAN. According to some embodiments, the RNAi is as defined in any one of the above embodiments and aspects. According to some embodiments, the RNAi is selected from siRNA and shRNA. According to some embodiments, the RNAi oligonucleotides are siRNA. According to some embodiments, the siRNA or shRNA inhibiting the expression of NCAN comprises a guide strand comprising a nucleic acid sequence selected from SEQ ID NO: 1-5, 11-15 and 86-95. Thus, in some embodiments, the present invention provides isolated EVs comprising siRNA or shRNA comprising a guide strand comprising a nucleic acid sequence selected from SEQ ID NO: 1-5, 11-15 and 86-95. According to some embodiments, the siRNA or shRNA comprises a guide strand consisting of a nucleic acid sequence selected from SEQ ID NO: 1-5, 11-15 and 86-95. According to some embodiments, the siRNA comprises a complementary strand. According to some embodiments, wherein the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises 14, 15, 16, 17, 18 or 19 nucleotides. According to some embodiments, the complementary strand comprises a nucleic acid sequence selected from SEQ ID NO: 6-10, 16-20, and 96-105 wherein the complementary strand comprises at positions 1 and 19 nucleic acids that are complementary to the nucleic acids at the corresponding positions in the sequence of the guide strand. According to one embodiment, siRNA or siRNA is conjugated with a hydrophobic moiety. According to some embodiments, the hydrophobic moiety is selected from the group consisting of a sterol, a ganglioside, a lipid, a vitamin, a fatty acid, a peptide, and a combination thereof. According to one embodiment, the siRNA or shRNA is conjugated with a sterol. In exemplary embodiments, the moiety is a sterol cholesterol molecule, therefore according to such embodiments, the siRNA or shRNA is conjugated with a cholesterol. According to one embodiment, siRNA or siRNA is conjugated with a hydrophilic moiety. According to some embodiments, the hydrophilic moiety is a carbohydrate. According to some embodiments, the carbohydrate is selected from a monosaccharide, disaccharide, trisaccharide, tetrasaccharide and oligosaccharide. According to some embodiments, the saccharide is selected from glucose, ribose, arabinose, galactose, mannose, sucrose and maltotriose. According to one embodiment, the siRNA or shRNA is conjugated with a carbohydrate. In exemplary embodiments, the moiety is a glucose molecule, therefore according to such embodiments, the siRNA or shRNA is conjugated with glucose.

According to some embodiments, the present invention provides isolated EVs loaded with siRNA molecules comprising a pair of oligonucleotides comprising or consisting of nucleic acid sequences (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the present invention provides isolated EVs loaded with siRNA molecules comprising a pair of oligonucleotides comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (xv) SEQ ID NOs: 95 and 105. According to some embodiments, the present invention provides isolated EVs loaded with siRNA molecules comprising a guide strand comprising the nucleic acid sequences SEQ ID NOs: 11 and a complementary strand comprising the nucleic acid sequences SEQ ID NO: 16. According to some embodiments, the present invention provides isolated EVs loaded with siRNA molecules comprising a guide strand comprising the nucleic acid sequences SEQ ID NOs: 12 and a complementary strand comprising the nucleic acid sequences SEQ ID NO: 17. According to some embodiments, the present invention provides isolated EVs loaded with siRNA molecules comprising a guide strand comprising the nucleic acid sequences SEQ ID NOs: 13 and a complementary strand comprising the nucleic acid sequences SEQ ID NO: 18. According to some embodiments, the present invention provides isolated EVs loaded with siRNA molecules comprising a guide strand and a complementary strand comprising the nucleic acid sequences SEQ ID NOs: 86 and 96, respectively. According to some embodiments, the present invention provides isolated EVs loaded with siRNA molecules comprising a guide strand and a complementary strand comprising the nucleic acid sequences SEQ ID NOs: 87 and 97, respectively. According to some embodiments, the present invention provides isolated EVs loaded with siRNA molecules comprising a guide strand and a complementary strand comprising the nucleic acid sequences SEQ ID NOs: 88 and 98, respectively. According to some embodiments, the present invention provides isolated EVs loaded with siRNA molecules comprising a guide strand and a complementary strand comprising the nucleic acid sequences SEQ ID NOs: 89 and 99, respectively. According to some embodiments, the present invention provides isolated EVs loaded with siRNA molecules comprising a guide strand and a complementary strand comprising the nucleic acid sequences SEQ ID NOs: 90 and 100, respectively. According to some embodiments, the present invention provides isolated EVs loaded with siRNA molecules comprising a guide strand and a complementary strand comprising the nucleic acid sequences SEQ ID NOs: 91 and 101, respectively. According to some embodiments, the present invention provides isolated EVs loaded with siRNA molecules comprising a guide strand and a complementary strand comprising the nucleic acid sequences SEQ ID NOs: 92 and 102, respectively. According to some embodiments, the present invention provides isolated EVs loaded with siRNA molecules comprising a guide strand and a complementary strand comprising the nucleic acid sequences SEQ ID NOs: 93 and 103, respectively. According to some embodiments, the present invention provides isolated EVs loaded with siRNA molecules comprising a guide strand and a complementary strand comprising the nucleic acid sequences SEQ ID NOs: 94 and 104, respectively. According to some embodiments, the present invention provides isolated EVs loaded with siRNA molecules comprising a guide strand and a complementary strand comprising the nucleic acid sequences SEQ ID NOs: 95 and 105, respectively. According to some embodiments, the siRNA is conjugated with cholesterol. According to some embodiments, the siRNA is conjugated with glucose. According to some embodiments, the EVs are exosomes, microvesicles or a combination thereof. According to some embodiments, the EVs are exosomes. According to some embodiments, the EVs are derived from mesenchymal stem cells. According to some embodiments, the EVs are derived from bone marrow MSCs. According to some embodiments, the EVs are exosomes.

According to some embodiments, the RNAi oligonucleotide inhibiting the expression of a protein NCAN is shRNA. According to some embodiments, the present invention provides isolated EVs loaded with shRNA inhibiting the expression of NCAN comprising a guide strand comprising or consisting of a nucleic acid sequence selected from SEQ ID NO: 1-5, 11-15, and 86-95. According to another embodiment, the shRNA comprises a complementary strand comprising a nucleic acid sequence selected from SEQ ID NO: 6-10, 16-20, and 96-105 wherein the complementary strand comprises nucleic acids at positions 1 and 19 that are complementary to the nucleic acids at the corresponding positions in the sequence of said guide strand. According to some embodiments, the shRNA inhibiting the expression of NCAN comprises a pair of oligonucleotides comprising or consisting of nucleic acid sequences (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the shRNA inhibiting expression of NCAN comprises a pair of oligonucleotides comprising or consisting of nucleic acid sequences (i) SEQ ID NO: 11 and 16; (ii) SEQ ID NO: 12 and 17; (iii) SEQ ID NO: 13 and 18; (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (xv) SEQ ID NOs: 95 and 105 According to some embodiments, the present invention provides isolated EVs loaded with shRNA molecules comprising a guide strand comprising the nucleic acid sequences SEQ ID NOs: 11 and a complementary strand comprising the nucleic acid sequences SEQ ID NO: 16. According to some embodiments, the present invention provides isolated EVs loaded with shRNA molecules comprising a guide strand comprising the nucleic acid sequences SEQ ID NOs: 12 and a complementary strand comprising the nucleic acid sequences SEQ ID NO: 17. According to some embodiments, the present invention provides isolated EVs loaded with shRNA molecules comprising a guide strand comprising the nucleic acid sequences SEQ ID NOs: 13 and a complementary strand comprising the nucleic acid sequences SEQ ID NO: 18. According to some embodiments, the present invention provides isolated EVs loaded with shRNA molecules comprising a guide strand and a complementary strand comprising the nucleic acid sequences SEQ ID NOs: 86 and 96, respectively. According to some embodiments, the present invention provides isolated EVs loaded with shRNA molecules comprising a guide strand and a complementary strand comprising the nucleic acid sequences SEQ ID NOs: 87 and 97, respectively. According to some embodiments, the present invention provides isolated EVs loaded with shRNA molecules comprising a guide strand and a complementary strand comprising the nucleic acid sequences SEQ ID NOs: 88 and 98, respectively. According to some embodiments, the present invention provides isolated EVs loaded with shRNA molecules comprising a guide strand and a complementary strand comprising the nucleic acid sequences SEQ ID NOs: 89 and 99, respectively. According to some embodiments, the present invention provides isolated EVs loaded with shRNA molecules comprising a guide strand and a complementary strand comprising the nucleic acid sequences SEQ ID NOs: 90 and 100, respectively. According to some embodiments, the present invention provides isolated EVs loaded with shRNA molecules comprising a guide strand and a complementary strand comprising the nucleic acid sequences SEQ ID NOs: 91 and 101, respectively. According to some embodiments, the present invention provides isolated EVs loaded with shRNA molecules comprising a guide strand and a complementary strand comprising the nucleic acid sequences SEQ ID NOs: 92 and 102, respectively. According to some embodiments, the present invention provides isolated EVs loaded with shRNA molecules comprising a guide strand and a complementary strand comprising the nucleic acid sequences SEQ ID NOs: 93 and 103, respectively. According to some embodiments, the present invention provides isolated EVs loaded with shRNA molecules comprising a guide strand and a complementary strand comprising the nucleic acid sequences SEQ ID NOs: 94 and 104, respectively. According to some embodiments, the present invention provides isolated EVs loaded with shRNA molecules comprising a guide strand and a complementary strand comprising the nucleic acid sequences SEQ ID NOs: 95 and 105, respectively. According to some embodiments, the shRNA is conjugated with cholesterol. According to some embodiments, the shRNA is conjugated with glucose. According to some embodiments, the EVs are exosomes, macrovesicles or a combination thereof. According to some embodiments, the EVs are exosomes. According to some embodiments, the EVs are derived from mesenchymal stem cells. According to some embodiments, the EVs are derived from bone marrow mesenchymal stem cells. According to some embodiments, the EVs are exosomes

According to some embodiments, the present invention provides isolated EVs loaded with RNA interference (RNAi) oligonucleotides, such as siRNA or shRNA, inhibiting the expression of Tenascin-R. According to some embodiments, the RNAi oligonucleotides are as defined in any one of the above embodiments. According to some embodiments, the RNAi oligonucleotides are selected from siRNA and shRNA. According to some embodiments, the RNAi oligonucleotides are siRNA. According to some embodiments, the siRNA or shRNA inhibiting the expression of TNR comprises a guide strand comprising a nucleic acid sequence selected from SEQ ID NO: 30-43 and 58-71. According to some embodiments, the present invention provides siRNA or shRNA comprising a guide strand comprising a nucleic acid sequence selected from SEQ ID NO: 30-43 and 58-71. According to some embodiments, the siRNA or shRNA comprises a guide strand consisting of a nucleic acid sequence selected from SEQ ID NO: 30-43 and 58-71. According to some embodiments, the siRNA comprises a complementary strand. According to some embodiments, wherein the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises 14, 15, 16, 17, 18 or 19 nucleotides. According to some embodiments, the complementary strand comprises a nucleic acid sequence selected from SEQ ID NO: 44-57 and 72-85, wherein the complementary strand comprises nucleic acids at positions 1 and 19 that are complementary to the nucleic acids at the corresponding positions in the sequence of said guide strand. According to one embodiment, siRNA or siRNA is conjugated with a hydrophobic moiety. According to some embodiments, the hydrophobic moiety is selected from the group consisting of a sterol, a ganglioside, a lipid, a vitamin, a fatty acid, a peptide, and a combination thereof. According to one embodiment, the siRNA or shRNA is conjugated with a sterol. In exemplary embodiments, the moiety is a sterol cholesterol molecule, therefore according to such embodiments, the siRNA or shRNA is conjugated with cholesterol. According to one embodiment, siRNA or siRNA is conjugated with a hydrophilic moiety. According to some embodiments, the hydrophilic moiety is a carbohydrate. According to some embodiments, the carbohydrate is selected from a monosaccharide, disaccharide, trisaccharide, tetrasaccharide and oligosaccharide. According to some embodiments, the saccharide is selected from glucose, ribose, arabinose, galactose, mannose, sucrose and maltotriose. According to one embodiment, the siRNA or shRNA is conjugated with a carbohydrate. In exemplary embodiments, the moiety is a glucose molecule, therefore according to such embodiments, the siRNA or shRNA is conjugated with glucose. According to some embodiments, the present invention provides isolated EVs loaded with siRNA molecules comprising a pair of oligonucleotides comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the present invention provides isolated EVs loaded with siRNA molecules comprising a pair of oligonucleotides comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (v) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides isolated EVs loaded with siRNA molecules comprising a guide strand comprising the nucleic acid sequences SEQ ID NOs: 58 and a complementary strand comprising the nucleic acid sequences SEQ ID NO: 72. According to some embodiments, the present invention provides isolated EVs loaded with siRNA molecules comprising a guide strand comprising the nucleic acid sequences SEQ ID NOs: 61 and a complementary strand comprising the nucleic acid sequences SEQ ID NO: 75. According to some embodiments, the present invention provides isolated EVs loaded with siRNA molecules comprising a guide strand comprising the nucleic acid sequences SEQ ID NOs: 62 and a complementary strand comprising the nucleic acid sequences SEQ ID NO: 76. According to some embodiments, the siRNA is conjugated with cholesterol. According to some embodiments, the siRNA is conjugated with glucose. According to some embodiments, the EVs are exosomes, macrovesicles or a combination thereof. According to some embodiments, the EVs are exosomes. According to some embodiments, the EVs are derived from mesenchymal stem cells. According to some embodiments, the EVs are derived from bone marrow mesenchymal stem cells.

According to some embodiments, the RNAi oligonucleotide inhibiting the expression of Tenascin-R is shRNA. According to some embodiments, the present invention provides isolated EVs loaded with shRNA inhibiting the expression of TNR comprising a guide strand comprising or consisting of a nucleic acid sequence selected from SEQ ID NO: 30-43 and 58-71. According to another embodiment, the shRNA comprises a complementary strand comprising a nucleic acid sequence selected from SEQ ID NO: 44-57 and 72-85 wherein the complementary strand comprises nucleic acids at positions 1 and 19 that are complementary to the nucleic acids at the corresponding positions in the sequence of said guide strand. According to some embodiments, the present invention provides isolated EVs loaded with shRNA molecules comprising a pair of oligonucleotides comprising nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the present invention provides isolated EVs loaded with shRNA molecules comprising a pair of oligonucleotides comprising nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (vi) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides isolated EVs loaded with shRNA molecules comprising a guide strand comprising the nucleic acid sequences SEQ ID NOs: 58 and a complementary strand comprising the nucleic acid sequences SEQ ID NO: 72. According to some embodiments, the present invention provides isolated EVs loaded with shRNA molecules comprising a guide strand comprising the nucleic acid sequences SEQ ID NOs: 61 and a complementary strand comprising the nucleic acid sequences SEQ ID NO: 75. According to some embodiments, the present invention provides isolated EVs loaded with shRNA molecules comprising a guide strand comprising the nucleic acid sequences SEQ ID NOs: 62 and a complementary strand comprising the nucleic acid sequences SEQ ID NO: 76. According to some embodiments, the shRNA is conjugated with cholesterol. According to some embodiments, the shRNA is conjugated with glucose. According to some embodiments, the EVs are exosomes, macrovesicles or a combination thereof. According to some embodiments, the EVs are derived from mesenchymal stem cells. According to some embodiments, the EVs are derived from bone marrow mesenchymal stem cells. According to some embodiments, the EVs are exosomes.

According to some embodiments, the present invention provides isolated EVs loaded with RNA interference (RNAi) oligonucleotides, such as siRNA or shRNA, inhibiting the expression of NCAN and TNR. According to some embodiments, the siRNA and/or shRNA molecules inhibiting the expression of NCAN and of TNR are as described hereinabove.

According to some embodiments, the isolated EVs of the present invention further comprise chondroitinase ABC or a nucleic acid molecule encoding thereof. According to some embodiments, the isolated EVs of the present invention further comprise a compound selected from, matrix metalloproteinases (MMPs), a disintegrin and metalloproteinase with thrombospondin motifs (ADAMTS), a nucleic acid molecule encoding and hyaluronic acid (HA).

Mesenchymal stem cell derived EVs may be produced by culturing mesenchymal stem cells in a medium to condition it.

The cells may be cultured for about 1, 2, 3, 4, 5, 6, 7, 8, 9, 10 days or more, for example, 3 days. The conditioned medium may be obtained by separating the cells from the medium. The conditioned medium may be centrifuged, for example at 500 xg. it may be concentrated by filtration through a membrane. The membrane may comprise a >1000 kDa membrane. The conditioned medium may be concentrated about 50 times or more.

It will be appreciated that polynucleotides or oligonucleotides such as siRNA or shRNA may also be loaded directly into the EVs. In one embodiment, direct loading of RNAi oligonucleotide to the EVs is carried out by electroporation and/or with the use of transfection agents. In alternative embodiments, the loading is carried out in the absence of electroporation and/or in the absence of transfection agents.

According to one embodiment, the EVs are incubated with the RNAi oligonucleotide inhibitor for a period of time sufficient to permit loading of the particles with the nucleic acid-based inhibitor. The duration of time sufficient to permit loading of the EVs with the nucleic acid-based inhibitor cargo can be optimized for the particular type of cargo and if modified to comprise a hydrophobic modification, then the type of modification. Generally, an incubation of about 1 hour or less is sufficient to permit efficient loading of particles with nucleic acid cargo. In many instances, hydrophobically modified cargo is efficiently loaded into exosomes in a very rapid period of time, for example, within 5 minutes. Accordingly, in some embodiments, efficient loading takes place during an incubation period of 5 minutes or less, e.g., from 1-5 minutes. In exemplary embodiments, efficient loading takes place during an incubation period of 5 minutes, 10 minutes, 15 minutes, 20 minutes, 30 minutes, etc. In other embodiments, efficient loading may take place within 1 hour, within 2 hours, within 3 hours, within 4 hours, within 5 hours, within 6 hours, within 7 hours, within 8 hours, within 9 hours, within 10 hours, within 12 hours, within 24 hours, 48 hours, etc.

Loading of EVs with oligonucleotides is not highly temperature dependent. In exemplary embodiments, exosomes are loaded at or around 37° C. In other embodiments, EVs (e.g. exosomes) can be loaded at or around room temperature. In other embodiments, exosomes can be loaded at or around 4° C.

According to some embodiments, the EVs can be loaded without the use of ultracentrifugation. According to other embodiment, the loading further comprises ultracentrifugation. According to some embodiment, the method of preparation further comprises a step of purification or isolation of the loaded EVs. According to one embodiment, the isolations are effected by centrifugation, e.g., ultracentrifugation. According to another embodiment, the isolations are effected via filtration. According to one embodiment, the ratio of EVs to residual parent cells following purification is at least 2, 3, 4, 5, 6, 8 or 10 times higher, or in certain advantageous embodiments at least 50, 100 or, 1000 times higher than in the initial material. According to some embodiment, the EVs are cell-free EVs.

According to some embodiments, the present invention provides a method of preparation of EVs, e.g., exosomes, the method comprises incubating EVs with conjugated RNAi oligonucleotides such as siRNA or shRNA for 0.5 to 5 hours at a temperature of 25 to 42° C. According to some embodiments, the conjugates of the siRNA or shRNA are conjugates with cholesterol. According to some embodiments, the conjugates of the siRNA or shRNA are conjugates with glucose.

According to one embodiment, the method further comprises a step of isolation of the loaded EVs using centrifugation, e.g., ultracentrifugation. According to some embodiments, another hydrophobic moiety may be used instead of cholesterol. According to one embodiment, the RNAi oligonucleotide is siRNA.

According to other embodiment, the EVs loaded with RNAi oligonucleotides of the present invention may be obtained from cells artificially loaded with a RNAi oligonucleotide or with a polynucleotide encoding and capable of expressing or generating of said RNAi inhibitor within a cell. In this case, the polynucleotide/oligonucleotide agent is ligated in a nucleic acid construct under the control of a cis-acting regulatory element (e.g. promoter) capable of directing an expression of the agent in a constitutive or inducible manner.

The nucleic acid agent may be delivered using an appropriate gene delivery vehicle/method (transfection, transduction, etc.). Optionally an appropriate expression system is used. Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co.

The expression construct may also be a virus. Examples of viral constructs include but are not limited to adenoviral vectors, retroviral vectors, vaccinia viral vectors, adeno-associated viral vectors, polyoma viral vectors, alphaviral vectors, rhabdoviral vectors, lentiviral vectors and herpesviral vectors.

A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction site and a translation termination sequence. By way of example, such constructs will typically include a 5′ LTR, a tRNA binding site, a packaging signal, an origin of second-strand DNA synthesis, and a 3′ LTR or a portion thereof.

The viral dose for infection may be at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles.

Double-stranded RNA may be synthesized by adding two opposing promoters to the ends of the gene segments, wherein one promoter is placed immediately 5′ to the gene and the opposing promoter is placed immediately 3′ to the gene segment. The dsRNA may then be transcribed with the appropriate polymerase.

In another embodiment, polynucleotide or oligonucleotide agents can be incubated with cells in culture, resulting in efficient uptake of the nucleic acid by cells. For such an embodiment, preferably the nucleic acid agents are hydrophobically modified, as further described herein below.

Irrespective of the method used to load the particles with the nucleic acid agents described herein, the cells are then incubated for a period of time sufficient for EVs, e.g. exosome, production. Exosomes isolated from the culture media contain exosomes loaded with the nucleic acid molecule taken up, produced or expressed by the cells. Accordingly, in one embodiment, a method of loading EVs with oligonucleotide cargo is provided, comprising incubating cells capable of EVs production (e.g. exosome production) with an oligonucleotide for a period of time sufficient for the oligonucleotide to be internalized by the cells, culturing the cells for a period of time sufficient for exosome secretion, and isolating exosomes loaded with the oligonucleotide from the culture medium.

According to some embodiments, the present invention provides isolated EVs prepared according to any one of the above embodiments.

According to another aspect, the present invention provides a composition comprising a plurality of the EVs according to any one of the above embodiments, and a carrier. According to some embodiments, the carrier is a pharmaceutically acceptable carrier and the composition is a pharmaceutical composition.

According to another aspect, the present invention provides a pharmaceutical composition comprising (i) the RNAi oligonucleotides of any one of the above aspects and embodiments, (ii) the isolated EVs of any one of the above aspects and embodiments, or (iii) the combination of (i) and (ii), and a pharmaceutically acceptable carrier. Any one of the above definitions, terms and embodiments are encompassed and apply herein as well. According to some embodiments, the present invention provides a pharmaceutical composition comprising isolated EVs loaded with RNA interference (RNAi) oligonucleotides inhibiting the expression of a protein selected from Neurocan (NCAN), Tenascin-R (TNR), Aggrecan (ACAN), Versican (VCAN), Brevican (BCAN) and a combination thereof. According to one embodiment, the present invention provides a pharmaceutical composition comprising isolated EVs loaded with RNA interference (RNAi) oligonucleotides inhibiting the expression of a protein selected from Neurocan, Tenascin-R and a combination thereof.

According to some embodiments, the present invention provides a pharmaceutical composition comprising anti-NCAN siRNA or shRNA as described in any one of the above aspects and embodiments, and a pharmaceutically acceptable carrier. According to some embodiments, the present invention provides a pharmaceutical composition comprising anti-TNR siRNA or shRNA as described in any one of the above aspects and embodiments, and a pharmaceutically acceptable carrier.

According to some embodiments, the present invention provides a pharmaceutical composition comprising isolated EVs loaded with RNAi oligonucleotides inhibiting the expression of the protein NCAN. According to some embodiments, the RNAi oligonucleotides are as defined in any one of the above embodiments. According to some embodiments, the RNAi oligonucleotides are selected from siRNA and shRNA. According to some embodiments, the RNAi oligonucleotides are siRNA. According to some embodiments, the siRNA or shRNA inhibiting the expression of NCAN comprises a guide strand comprising a nucleic acid sequence selected from SEQ ID NO: 1-5, 11-15, and 86-95. According to some embodiments, the siRNA or shRNA comprises a guide strand consisting of a nucleic acid sequence selected from SEQ ID NO: 1-5, 11-15, and 86-95. According to some embodiments, the siRNA comprises a complementary strand. According to some embodiments, the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises 14, 15, 16, 17, 18 or 19 nucleotides. According to some embodiments, the complementary strand comprises a nucleic acid sequence selected from SEQ ID NO: 6-10, 16-20 and 96-105, wherein the complementary strand comprises nucleic acids at positions 1 and 19 that are complementary to the nucleic acids at the corresponding positions in the sequence of said guide strand. According to one embodiment, siRNA or siRNA is conjugated with a hydrophobic moiety. According to some embodiments, the hydrophobic moiety is selected from the group consisting of a sterol, a ganglioside, a lipid, a vitamin, a fatty acid, a peptide, and a combination thereof. According to one embodiment, the siRNA or shRNA is conjugated with a sterol. In exemplary embodiments, the moiety is a sterol cholesterol molecule, therefore according to such embodiments, the siRNA or shRNA is conjugated with a cholesterol. According to one embodiment, siRNA or siRNA is conjugated with a hydrophilic moiety. According to some embodiments, the hydrophilic moiety is a carbohydrate. According to some embodiments, the carbohydrate is selected from a monosaccharide, disaccharide, trisaccharide, tetrasaccharide and oligosaccharide. According to some embodiments, the saccharide is selected from glucose, ribose, arabinose, galactose, mannose, sucrose and maltotriose. According to one embodiment, the siRNA or shRNA is conjugated with a carbohydrate. In exemplary embodiments, the moiety is a glucose molecule, therefore according to such embodiments, the siRNA or shRNA is conjugated with glucose.

According to some embodiments, the present invention provides a pharmaceutical composition comprising isolated EVs loaded with siRNA molecules comprising a pair of oligonucleotides comprising or consisting of nucleic acid sequences (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the present invention provides a pharmaceutical composition comprising isolated EVs loaded with siRNA molecules comprising a pair of oligonucleotides comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (xv) SEQ ID NOs: 95 and 105. According to some embodiments, the present invention provides a pharmaceutical composition comprising isolated EVs loaded with siRNA molecules comprising a guide strand comprising the nucleic acid sequences SEQ ID NOs: 11 and a complementary strand comprising the nucleic acid sequences SEQ ID NO: 16. According to some embodiments, the present invention provides a pharmaceutical composition comprising isolated EVs loaded with siRNA molecules comprising a guide strand comprising the nucleic acid sequences SEQ ID NOs: 12 and a complementary strand comprising the nucleic acid sequences SEQ ID NO: 17. According to some embodiments, the present invention provides a pharmaceutical composition comprising isolated EVs loaded with siRNA molecules comprising a guide strand comprising the nucleic acid sequences SEQ ID NOs: 13 and a complementary strand comprising the nucleic acid sequences SEQ ID NO: 18. According to some embodiments, the present invention provides a pharmaceutical composition comprising isolated EVs loaded with siRNA molecules comprising a guide strand and a complementary strand comprising the nucleic acid sequences SEQ ID NOs: 86 and 96, respectively. According to some embodiments, the present invention provides a pharmaceutical composition comprising isolated EVs loaded with siRNA molecules comprising a guide strand and a complementary strand comprising the nucleic acid sequences SEQ ID NOs: 87 and 97, respectively. According to some embodiments, the present invention provides a pharmaceutical composition comprising isolated EVs loaded with siRNA molecules comprising a guide strand and a complementary strand comprising the nucleic acid sequences SEQ ID NOs: 88 and 98, respectively. According to some embodiments, the present invention provides a pharmaceutical composition comprising isolated EVs loaded with siRNA molecules comprising a guide strand and a complementary strand comprising the nucleic acid sequences SEQ ID NOs: 89 and 99, respectively. According to some embodiments, the present invention provides a pharmaceutical composition comprising isolated EVs loaded with siRNA molecules comprising a guide strand and a complementary strand comprising the nucleic acid sequences SEQ ID NOs: 90 and 100, respectively. According to some embodiments, the present invention provides a pharmaceutical composition comprising isolated EVs loaded with siRNA molecules comprising a guide strand and a complementary strand comprising the nucleic acid sequences SEQ ID NOs: 91 and 101, respectively. According to some embodiments, the present invention provides a pharmaceutical composition comprising isolated EVs loaded with siRNA molecules comprising a guide strand and a complementary strand comprising the nucleic acid sequences SEQ ID NOs: 92 and 102, respectively. According to some embodiments, the present invention provides a pharmaceutical composition comprising isolated EVs loaded with siRNA molecules comprising a guide strand and a complementary strand comprising the nucleic acid sequences SEQ ID NOs: 93 and 103, respectively. According to some embodiments, the present invention provides a pharmaceutical composition comprising isolated EVs loaded with siRNA molecules comprising a guide strand and a complementary strand comprising the nucleic acid sequences SEQ ID NOs: 94 and 104, respectively. According to some embodiments, the present invention provides a pharmaceutical composition comprising isolated EVs loaded with siRNA molecules comprising a guide strand and a complementary strand comprising the nucleic acid sequences SEQ ID NOs: 95 and 105, respectively. According to some embodiments, the siRNA is conjugated with cholesterol. According to some embodiments, the siRNA is conjugated with glucose. According to some embodiments, the EVs are exosomes, macrovesicles or a combination thereof. According to some embodiments, the EVs are derived from mesenchymal stem cells.

According to some embodiments, the present invention provides a pharmaceutical composition comprising isolated EVs loaded with shRNA molecules comprising a pair of oligonucleotides comprising or consisting of nucleic acid sequences (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the present invention provides a pharmaceutical composition comprising isolated EVs loaded with shRNA molecules comprising a pair of oligonucleotides comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOS: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOS: 92 and 102; (xiii) SEQ ID NOs: 93 and 103; (xiv) SEQ ID NOs: 94 and 104; or (xv) SEQ ID NOs: 95 and 105. According to some embodiments, the shRNA is conjugated with cholesterol. According to some embodiments, the shRNA is conjugated with glucose. According to some embodiments, the EVs are exosomes, macrovesicles or a combination thereof. According to some embodiments, the EVs are derived from mesenchymal stem cells.

According to some embodiments, the present invention provides a pharmaceutical composition comprising isolated EVs loaded with RNAi oligonucleotides inhibiting the expression of the protein TNR. According to some embodiments, the RNAi oligonucleotides are as defined in any one of the above embodiments. According to some embodiments, the RNAi is selected from siRNA and shRNA. According to some embodiments, the RNAi oligonucleotides are siRNA. According to some embodiments, the siRNA or shRNA inhibiting the expression of TNR comprises a guide strand comprising a nucleic acid sequence selected from SEQ ID NO: 30-43 and 58-71. According to some embodiments, the siRNA or shRNA comprises a guide strand consisting of a nucleic acid sequence selected from SEQ ID NO: 30-43 and 58-71. According to some embodiments, the siRNA comprises a complementary strand, i.e. a strand complementary to said guide strand. According to some embodiments, wherein the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises 14, 15, 16, 17, 18 or 19 nucleotides. According to some embodiments, the complementary strand comprises a nucleic acid sequence selected from SEQ ID NO: 44-57 and 72-85, wherein the complementary strand comprises nucleic acids at positions 1 and 19 that are complementary to the nucleic acids at the corresponding positions in the sequence of said guide strand. According to one embodiment, siRNA or siRNA inhibiting the expression of TNR is conjugated with a hydrophobic moiety. According to some embodiments, the hydrophobic moiety is selected from the group consisting of a sterol, a ganglioside, a lipid, a vitamin, a fatty acid, a peptide, and a combination thereof. According to one embodiment, the siRNA or shRNA is conjugated with a sterol. In exemplary embodiments, the moiety is a sterol cholesterol molecule, therefore according to such embodiments, the siRNA or shRNA is conjugated with a cholesterol. According to one embodiment, siRNA or siRNA is conjugated with a hydrophilic moiety. According to some embodiments, the hydrophilic moiety is a carbohydrate. According to some embodiments, the carbohydrate is selected from a monosaccharide, disaccharide, trisaccharide, tetrasaccharide and oligosaccharide. According to some embodiments, the saccharide is selected from glucose, ribose, arabinose, galactose, mannose, sucrose and maltotriose. According to one embodiment, the siRNA or shRNA is conjugated with a carbohydrate. In exemplary embodiments, the moiety is a glucose molecule, therefore according to such embodiments, the siRNA or shRNA is conjugated with glucose.

According to some embodiments, the present invention provides a pharmaceutical composition comprising EVs loaded with siRNA molecules inhibiting the expression of TNR comprising a guide strand comprising or consisting of a nucleic acid sequence selected from SEQ ID NO: 30-43 and 58-71. According to another embodiment, the siRNA comprises a complementary strand comprising a nucleic acid sequence selected from SEQ ID NO: 44-57 and 72-85 wherein the complementary strand comprises nucleic acids at positions 1 and 19 that are complementary to the nucleic acids at the corresponding positions in the sequence of said guide strand. According to some embodiments, the present invention provides a pharmaceutical composition comprising isolated EVs loaded with siRNA molecules comprising a pair of oligonucleotides comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the present invention provides a pharmaceutical composition comprising isolated EVs loaded with siRNA molecules comprising a pair of oligonucleotides comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (vi) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides a pharmaceutical composition comprising isolated EVs loaded with siRNA molecules comprising a guide strand comprising the nucleic acid sequences SEQ ID NOs: 58 and a complementary strand comprising the nucleic acid sequences SEQ ID NO: 72. According to some embodiments, the present invention provides a pharmaceutical composition comprising isolated EVs loaded with siRNA molecules comprising a guide strand comprising the nucleic acid sequences SEQ ID NOs: 61 and a complementary strand comprising the nucleic acid sequences SEQ ID NO: 75. According to some embodiments, the present invention provides a pharmaceutical composition comprising isolated EVs loaded with siRNA molecules comprising a guide strand comprising the nucleic acid sequences SEQ ID NOs: 62 and a complementary strand comprising the nucleic acid sequences SEQ ID NO: 76. According to some embodiments, the siRNA is conjugated with cholesterol. According to some embodiments, the siRNA is conjugated with glucose. According to some embodiments, the EVs are exosomes, microvesicles or a combination thereof. According to some embodiments, the EVs are derived from mesenchymal stem cells.

According to some embodiments, the present invention provides a pharmaceutical composition comprising isolated EVs loaded with shRNA molecules comprising a pair of oligonucleotides comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the present invention provides a pharmaceutical composition comprising isolated EVs loaded with shRNA molecules comprising a pair of oligonucleotides comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (v) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides a pharmaceutical composition comprising isolated EVs loaded with shRNA molecules comprising a guide strand comprising the nucleic acid sequences SEQ ID NOs: 58 and a complementary strand comprising the nucleic acid sequences SEQ ID NO: 72. According to some embodiments, the present invention provides a pharmaceutical composition comprising isolated EVs loaded with shRNA molecules comprising a guide strand comprising the nucleic acid sequences SEQ ID NOS: 61 and a complementary strand comprising the nucleic acid sequences SEQ ID NO: 75. According to some embodiments, the present invention provides a pharmaceutical composition comprising isolated EVs loaded with shRNA molecules comprising a guide strand comprising the nucleic acid sequences SEQ ID NOs: 62 and a complementary strand comprising the nucleic acid sequences SEQ ID NO: 76. According to some embodiments, the siRNA is conjugated with cholesterol. According to some embodiments, the siRNA is conjugated with glucose. According to some embodiments, the EVs are exosomes, microvesicles or a combination thereof. According to some embodiments, the EVs are derived from mesenchymal stem cells.

According to some embodiments, the present invention provides a pharmaceutical composition comprising isolated EVs loaded with RNAi oligonucleotides of the present invention inhibiting the expression of the protein NCAN and EVs loaded with RNAi oligonucleotides of present invention inhibiting the expression of the protein TNR.

The siRNA and shRNA molecules promote sequence-specific degradation of mRNA by RNAi to achieve inhibition of the expression of the desired protein, e.g., NCAN or TNR, or reduction of the expression level of the desired gene, e.g., NCAN or TNR by 95%, 90%, 85%, 80%, 75%, 70%, 60%, 50%, 40%, 30%, 20%, 10%, or 5%.

The term “pharmaceutical composition” as used herein refers to a composition comprising siRNA, shRNA or EVs loaded with the RNAi of the present invention, in particular EVs such as exosomes, formulated together with one or more pharmaceutically acceptable carriers.

Formulation of the pharmaceutical composition may be adjusted according to applications. In particular, the pharmaceutical composition may be formulated using a method known in the art so as to provide a rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, sprays, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. solid carriers or excipients such as, for example, lactose, starch or talcum or liquid carriers such as, for example, water, fatty oils or liquid paraffins. Other examples of the carrier include culture medium such as DMEM or RPMI; hypothermic storage medium containing components that scavenge free radicals, provide pH buffering, oncotic/osmotic support, energy substrates and ionic concentrations that balance the intracellular state at low temperatures; and mixtures of organic solvents with water.

According to any one of the above embodiments, the pharmaceutical composition is formulated for administration via an administration route selected from intranasal, intra-lesion, intrathecal, intravenous, intramuscular, subcutaneous, sublingual, oral, and intracerebral administration routes. According to one embodiment, the pharmaceutical composition is formulated for intranasal administration. According to some embodiment, such pharmaceutical composition is in a form of liquid solution, nasal drops, spray, measured stray or powder. According to other embodiment, the pharmaceutical composition is formulated for injection, e.g., intra-lesion, intrathecal or intravenous injection. According to such embodiments, the pharmaceutical composition is in a form of sterile solution of injection.

According to some embodiments, the pharmaceutical composition is formulated for administration via an administration route selected from intranasal, intra-lesion, intrathecal, intravenous, intramuscular, subcutaneous, sublingual, oral, and intracerebral administration routes.

According to one embodiment, the pharmaceutical composition is formulated for intranasal administration.

According to some embodiments, the present invention provides an intranasal pharmaceutical composition comprising EVs loaded with siRNA or shRNA molecules inhibiting the expression of NCAN comprising a guide strand comprising or consisting of a nucleic acid sequence selected from SEQ ID NO: 1-5, 11-15 and 86-95, wherein the complementary strand comprises nucleic acids at positions 1 and 19 that are complementary to the nucleic acids at the corresponding positions in the sequence of said guide strand. According to another embodiment, the siRNA or shRNA inhibiting the expression of NCAN comprises a complementary strand comprising a nucleic acid sequence selected from SEQ ID NO: 6-10, 16-20 and 96-105, wherein the complementary strand comprises nucleic acids at positions 1 and 19 that are complementary to the nucleic acids at the corresponding positions in the sequence of said guide strand. According to some embodiments, the present invention provides an intranasal pharmaceutical composition comprising EVs loaded with siRNA or shRNA molecules inhibiting the expression of NCAN comprising a guide strand comprising or consisting of nucleic acid sequences from (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the present invention provides an intranasal pharmaceutical composition comprising EVs loaded with siRNA or shRNA molecules inhibiting the expression of NCAN comprising a guide strand comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (Xv) SEQ ID NOs: 95 and 105. According to some embodiments, the present invention provides an intranasal pharmaceutical composition comprising isolated EVs loaded with siRNA molecules comprising a guide strand and a complementary strand comprising the nucleic acid sequences SEQ ID NOs: 11 and 16, respectively. According to some embodiments, the present invention provides an intranasal pharmaceutical composition comprising isolated EVs loaded with siRNA molecules comprising a guide strand and a complementary strand comprising the nucleic acid sequences SEQ ID NOs: 12 and 17, respectively. According to some embodiments, the present invention provides an intranasal pharmaceutical composition comprising isolated EVs loaded with siRNA molecules comprising a guide strand and a complementary strand comprising the nucleic acid sequences SEQ ID NOs: 13 and 18, respectively. According to some embodiments, the siRNA or shRNA is conjugated with cholesterol. According to some embodiments, the siRNA or shRNA is conjugated with glucose. According to some embodiments, the EVs are exosomes, microvesicles or a combination thereof. According to some embodiments, the EVs are derived from mesenchymal stem cells.

According to some embodiments, the present invention provides an intranasal pharmaceutical composition comprising EVs loaded with siRNA or shRNA molecules inhibiting the expression of TNR comprising a guide strand comprising or consisting of a nucleic acid sequence selected from SEQ ID NO: 30-43 and 58-71. According to another embodiment, the siRNA or shRNA comprises a complementary strand comprising a nucleic acid sequence selected from SEQ ID NO: 44-57 and 72-85 wherein the complementary strand comprises nucleic acids at positions 1 and 19 that are complementary to the nucleic acids at the corresponding positions in the sequence of said guide strand. According to some embodiments, the present invention provides an intranasal pharmaceutical composition comprising EVs loaded with siRNA or shRNA molecules inhibiting the expression of TNR comprising a guide strand comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the present invention provides an intranasal pharmaceutical composition comprising EVs loaded with siRNA or shRNA molecules inhibiting the expression of TNR comprising a guide strand comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (v) SEQ ID NO: 63 and 77;
(vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the present invention provides an intranasal pharmaceutical composition comprising isolated EVs loaded with siRNA molecules comprising a guide strand comprising the nucleic acid sequences SEQ ID NOs: 58 and a complementary strand comprising the nucleic acid sequences SEQ ID NO: 72. According to some embodiments, the present invention provides an intranasal pharmaceutical composition comprising isolated EVs loaded with siRNA molecules comprising a guide strand comprising the nucleic acid sequences SEQ ID NOS: 61 and a complementary strand comprising the nucleic acid sequences SEQ ID NO: 75. According to some embodiments, the present invention provides an intranasal pharmaceutical composition comprising isolated EVs loaded with siRNA molecules comprising a guide strand comprising the nucleic acid sequences SEQ ID NOs: 62 and a complementary strand comprising the nucleic acid sequences SEQ ID NO: 76. According to some embodiments, the siRNA or shRNA is conjugated with cholesterol. According to some embodiments, the siRNA or shRNA is conjugated with glucose According to some embodiments, the EVs are exosomes, microvesicles or a combination thereof. According to some embodiments, the EVs are derived from mesenchymal stem cells.

According to some embodiments, the isolated EVs of the present invention further comprise chondroitinase ABC (chABC) or a nucleic acid molecule encoding thereof. According to some embodiments, the isolated EVs of the present invention further comprise a compound selected from, matrix metalloproteinases (MMPs), a disintegrin and metalloproteinase with thrombospondin motifs (ADAMTS), a nucleic acid molecule encoding thereof and hyaluronic acid (HA).

According to any one of the above embodiments, the pharmaceutical composition of the present invention is for use in inducing neuroregeneration after neural damage. According to any one of the above embodiments, the pharmaceutical composition of the present invention is for use in prevention of the inhibition of neuroregeneration after neural damage. According to any one of the above embodiments, the pharmaceutical composition of the present invention is for use in treating neurological disease, disorder, damage or condition. According to one embodiment, the neurological condition is spinal cord injury.

The pharmaceutical composition according to any one of the above embodiments, is for use in treating neurological disease, disorder or condition. According to some embodiments, the pharmaceutical composition is for use in treating neuronal injury or damage in a subject.

The term “neurological disease, disorder or condition” refers to a disease, disorder or condition of the brain, spine and/or the nerves that connect them.

According to a particular embodiment, the condition is due to an injury. According to one embodiment, the injury is to spinal cord, i.e., spinal cord injury (SCI). According to other embodiment, the neurological disease, disorder or condition is a neuronal damage.

The terms “spinal cord injury” and “SCI” are used herein interchangeably and refer to an injury to the spinal cord. According to one embodiment, the injury is a result of a trauma. According to another embodiment, the injury or a damage is a result of a degeneration or a disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. Thus, according to one embodiment, the SCI is selected from a complete and incomplete SCI. According to some embodiment, the spinal cord injury is selected from an acute or chronic SCI. The spinal cord injury may be susceptible to secondary tissue injury, including but not limited to: glial scarring, myelin inhibition, demyelination, cell death, lack of neurotrophic support, ischemia, free-radical formation, and excitotoxicity.

Diseases of the spinal cord include but are not limited to autoimmune diseases (e.g. multiple sclerosis), inflammatory diseases (e.g. Arachnoiditis), neurodegenerative diseases, polio, spina bifida and spinal tumors.

Subjects that may be treated according to the teaching of the present invention include mammalian subjects, such as humans, mice, rats, monkeys, dogs and cats. In one embodiment, the subject is a human subject.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s). According to some embodiments, the term “treating” comprises neural regeneration, axonal propagation, decreased astrogliosis and microgliosis at the injury site. According to other embodiments, the term encompasses improvement in symptoms associated with the disease or condition. According to one embodiment, the term “treating” comprises improvement in locomotor parameters. According to one embodiment, improvement in locomotor parameters comprises improvement in 20%, 30%, 40%, 50%, 60%, 70%, 80% or 90% of locomotor parameters in comparison to untreated subject. According to some embodiment, treating comprises reducing astrogliosis and/or microgliosis at the injury site. According to one embodiment, reducing astrogliosis and/or microgliosis comprises reduction of 10%, 20%, 30%, 40%, 50%, 60%, 70%, or 80% of astrogliosis and/or microgliosis in comparison to untreated subject.

The pharmaceutical composition of the present invention may be administered using any known method. The terms “administering” or “administration of” a substance, a compound or an agent to a subject can be carried out using one of a variety of methods known to those skilled in the art. For example, a compound or an agent can be administered intranasally (e.g., by inhalation), intrathecally (into the spinal canal, or into the subarachnoid space), arterially, intradermally, intramuscularly, intraperitoneally, intravenously, subcutaneously, ocularly, sublingually, orally (by ingestion), intracerebrally, and trans-dermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administrations, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the pharmaceutical composition of the present invention is administered intranasally. According to another embodiment, the pharmaceutical composition of the present invention is administered intra-lesion. According to another embodiment, the pharmaceutical composition of the present invention is administered in proximity to the damage or injury. According to one embodiment, the pharmaceutical composition is administered orally.

According to one embodiment, the pharmaceutical composition is administered intranasally.

An exemplary dose of membrane vesicles (e.g. exosomes) that may be administered (e.g. intranasally) per treatment may be between 1×10⁶-1×10²⁰ and or between 1×10⁹-1×10¹⁵ for a 70 kg human.

According to some embodiments, the pharmaceutical composition according to any one of the above embodiments, is for use in enhancing neural regeneration. According to some embodiments, the pharmaceutical composition according to any one of the above embodiments, is for use in enhancing the plasticity of neurons.

According to another aspect, the present invention provides a method of treating a neuronal injury or damage in a subject in need thereof, comprising administering to the subject a therapeutically effective amount of isolated EVs comprising an inhibitor of expression of a protein selected from Neurocan (NCAN), Tenascin-R (TNR), Aggrecan (ACAN), Versican (VCAN), Brevican (BCAN) and a combination thereof. According to some embodiments, the present invention provides a method of inducing neuroregeneration after a neural damage in a subject in need thereof, comprising administering to the subject a therapeutically effective amount of isolated EVs comprising an inhibitor of expression of a protein selected from Neurocan (NCAN), Tenascin-R (TNR), Aggrecan (ACAN), Versican (VCAN), Brevican (BCAN) and a combination thereof. According to some embodiments, wherein the administering is intranasal. According to other embodiments, the method further comprises administering chondroitinase ABC. According to other embodiments, the method further comprises administering matrix metalloproteinases (MMPs), a disintegrin metalloproteinase with thrombospondin motifs (ADAMTS), or HA.

The term “therapeutically effective amount” of the EVs, when administered to a subject will have the intended therapeutic effect, e.g. treating neuronal injury or damage such as SCI. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation.

In another aspect, the present disclosure provides a method for inhibiting or reducing the expression level of the NCAN gene in a cell in vivo or in vitro, comprising introducing into the cell the siRNA or shRNA molecules, the EVs or the pharmaceutical composition according to any of the above embodiments and aspects, such that the expression level of the NCAN gene is inhibited or reduced by at least 95%, at least 90%, at least 85%, at least 80%, at least 75%, at least 70%, at least 60%, at least 50%, at least 40%, at least 30%, at least 20%, at least 10%, or at least 5%. According to some embodiments, the siRNA or shRNA inhibiting the expression of NCAN molecules comprise a guide strand comprising or consisting of a nucleic acid sequence selected from SEQ ID NO: 1-5, 11-15 and 86-95. According to another embodiment, the siRNA or shRNA comprises a complementary strand comprising a nucleic acid sequence selected from SEQ ID NO: 6-10, 16-20 and 96-105 wherein the complementary strand comprises nucleic acids at positions 1 and 19 that are complementary to the nucleic acids at the corresponding positions in the sequence of said guide strand. According to some embodiments, the siRNA or shRNA inhibiting the expression of NCAN comprises a pair of oligonucleotides comprising or consisting of nucleic acid sequences (i) SEQ ID NO: 1 and 6; (ii) SEQ ID NO: 2 and 7; (iii) SEQ ID NO: 3 and 8; or (iv) SEQ ID NO: 4 and 9; (v) SEQ ID NO: 5 and 10, wherein N nucleotides in the guide strand are complementary to N in the complementary strand at the corresponding positions. According to some embodiments, the siRNA or shRNA inhibiting the expression of NCAN comprises a pair of oligonucleotides comprising or consisting of nucleic acid sequences (i) SEQ ID NO: 11 and 16; (ii) SEQ ID NO: 12 and 17; (iii) SEQ ID NO: 13 and 18; or (iv) SEQ ID NO: 14 and 9; (v) SEQ ID NO: 15 and 20; (vi) SEQ ID NOs: 86 and 96; (vii) SEQ ID NOs: 87 and 77; (viii) SEQ ID NO: 88 and 88; (ix) SEQ ID NOs: 89 and 99; (x) SEQ ID NOs: 90 and 100; (xi) SEQ ID NOs: 91 and 101; (xii) SEQ ID NOs: 92 and 102; (xiii) SEQ ID NOs: 93 and 103; (xiv) SEQ ID NOs: 94 and 104; or (xv) SEQ ID NOs: 95 and 105. According to some embodiments, the siRNA or shRNA inhibiting the expression of NCAN comprises a pair of oligonucleotides comprising or consisting of nucleic acid sequences SEQ ID NO: 11 and 16. According to some embodiments, the siRNA or shRNA inhibiting the expression of NCAN comprises a pair of oligonucleotides comprising or consisting of nucleic acid sequences SEQ ID NO: 12 and 17. According to some embodiments, the siRNA or shRNA inhibiting the expression of NCAN comprises a pair of oligonucleotides comprising or consisting of nucleic acid sequences SEQ ID NO: 13 and 18. According to some embodiments, the siRNA or shRNA is conjugated with cholesterol. According to some embodiments, the siRNA or shRNA is conjugated with glucose

In another aspect, the present disclosure provides a method for inhibiting or reducing the expression level of the TNR gene in a cell in vivo or in vitro, comprising introducing into the cell the siRNA or shRNA molecules, the EVs or the pharmaceutical composition according to any of the above embodiments and aspects, such that the expression level of the TNR gene is inhibited or reduced by at least 95%, at least 90%, at least 85%, at least 80%, at least 75%, at least 70%, at least 60%, at least 50%, at least 40%, at least 30%, at least 20%, at least 10%, or at least 5%. According to some embodiments, the siRNA or shRNA inhibiting the expression of TNR molecules comprise a guide strand comprising or consisting of a nucleic acid sequence selected from SEQ ID NO: 30-43 and 58-71. According to another embodiment, the siRNA or shRNA inhibiting the expression of TNR comprises a complementary strand comprising a nucleic acid sequence selected from SEQ ID NO: 44-57 and 72-85 wherein the complementary strand comprises nucleic acids at positions 1 and 19 that are complementary to the nucleic acids at the corresponding positions in the sequence of said guide strand. According to some embodiments, the siRNA or shRNA inhibiting the expression of TNR comprises a pair of oligonucleotides comprising or consisting of nucleic acid sequences (i) SEQ ID NO: 30 and 44; (ii) SEQ ID NO: 31 and 45; (iii) SEQ ID NO: 32 and 46 (iv) SEQ ID NO: 33 and 47; (v) SEQ ID NO: 34 and 48; (vi) SEQ ID NO: 45 and 49; (vii) SEQ ID NO: 56 and 50; (viii) SEQ ID NO: 37 and 51; (ix) SEQ ID NO: 38 and 52; (x) SEQ ID NO: 39 and 53; (xi) SEQ ID NO: 40 and 54; (xii) SEQ ID NO: 41 and 55; (xiii) SEQ ID NO: 42 and 56; or (xiv) SEQ ID NO: 43 and 57, wherein N nucleotides in the guide strand are complementary to N in the complementary strand at the corresponding positions. According to some embodiments, the siRNA or shRNA inhibiting the expression of TNR comprises a pair of oligonucleotides comprising or consisting of nucleic acid sequences (i) SEQ ID NO: 58 and 72; (ii) SEQ ID NO: 59 and 73; (iii) SEQ ID NO: 60 and 74; (iv) SEQ ID NO: 61 and 75; (v) SEQ ID NO: 62 and 76; (v) SEQ ID NO: 63 and 77; (vii) SEQ ID NO: 64 and 78; (viii) SEQ ID NO: 65 and 79; (ix) SEQ ID NO: 66 and 80; (x) SEQ ID NO: 67 and 81; (xi) SEQ ID NO: 68 and 82; (xii) SEQ ID NO: 69 and 83; (xiii) SEQ ID NO: 70 and 84; or (xiv) SEQ ID NO: 71 and 85. According to some embodiments, the siRNA or shRNA inhibiting the expression of TNR comprises a pair of oligonucleotides comprising or consisting of nucleic acid sequences SEQ ID NOs: 58 and 72. According to some embodiments, the siRNA or shRNA inhibiting the expression of TNR comprises a pair of oligonucleotides comprising or consisting of nucleic acid sequences SEQ ID NOs: 61 and 75. According to some embodiments, the siRNA or shRNA inhibiting the expression of TNR comprises a pair of oligonucleotides comprising or consisting of nucleic acid sequences SEQ ID NOs: 62 and 76. According to some embodiments, the siRNA or shRNA is conjugated with cholesterol. According to some embodiments, the siRNA or shRNA is conjugated with glucose

TABLE 1
Sequences of the present invention

| | | | |
|---|---|---|---|
| | Antisense | | |
| | (Guide) | | |
| siRNA duplex | (A)/ | SEQ-ID | |
| ID (name) | Sense (S) | No | Sequence (5′-3′) |

Anti-NCAN

| | | | |
|---|---|---|---|
| sirna_3816-g | A | 1 | NACAGUGGCAUGGACAUUNUA |
| | S | 6 | NAAUGUCCAUGCCACUGUN |
| sirna_1172-g | A | 2 | NGAGCUCGGAAGCAGUAG NCG |
| | S | 7 | NCUACUGCUUCCGAGCUC N |
| sirna_632-g | A | 3 | NGGGCCUCAGCGAAGGUC NGU |
| | S | 8 | NGACCUUCGCUGAGGCCC N |
| sirna_1014-g | A | 4 | NGGGUCGCACUGGUCCAG NCC |
| | S | 9 | NCUGGACCAGUGCGACCC N |
| sirna_55-g | A | 5 | NCCGGGCACAAAGGACGC NGC |
| | S | 10 | NGCGUCCUUUGUGCCCGG N |
| sirna_3816G | A | 11 | UACAGUGGCAUGGACAUUCUA |
| | S | 16 | GAAUGUCCAUGCCACUGUA |
| sirna_1172GA | A | 12 | UGAGCUCGGAAGCAGUAGCCG |
| | S | 17 | GCUACUGCUUCCGAGCUCA |
| sirna_632GA | A | 13 | UGGGCCUCAGCGAAGGUCCGU |
| | S | 18 | GGACCUUCGCUGAGGCCCA |
| sirna_1014GA | A | 14 | UGGGUCGCACUGGUCCAGCCC |
| | S | 19 | GCUGGACCAGUGCGACCCA |
| sirna_55GA | A | 15 | UCCGGGCACAAAGGACGCCGC |
| | S | 20 | GGCGUCCUUUGUGCCCGGA |
| sirna_3816GT | A | 86 | AACAGUGGCAUGGACAUUCua |
| | S | 96 | GAATGTCCATGCCACTGTT |
| sirna_3816T | A | 87 | AACAGUGGCAUGGACAUUGua |
| | S | 97 | CAATGTCCATGCCACTGTT |
| sirna_1172 | A | 88 | UGAGCUCGGAAGCAGUAGGcg |
| | S | 98 | CCTACTGCTTCCGAGCTCA |
| sirna_1172GT | A | 89 | AGAGCUCGGAAGCAGUAGCcg |
| | S | 99 | GCTACTGCTTCCGAGCTCT |
| sirna_1172T | A | 90 | AGAGCUCGGAAGCAGUAGGcg |
| | S | 100 | CCTACTGCTTCCGAGCTCT |
| sirna_632GT | A | 91 | AGGGCCUCAGCGAAGGUCCgu |
| | S | 101 | GGACCTTCGCTGAGGCCCT |
| sirna_632C | A | 92 | UGGGCCUCAGCGAAGGUCGgu |
| | S | 102 | CGACCTTCGCTGAGGCCCA |
| sirna_632CT | A | 93 | AGGGCCUCAGCGAAGGUCGgu |
| | S | 103 | CGACCTTCGCTGAGGCCCT |
| sirna_632 | A | 94 | UGGGCCUCAGCGAAGGUCAgu |
| | S | 104 | TGACCTTCGCTGAGGCCCA |
| sirna_632T | A | 95 | AGGGCCUCAGCGAAGGUCAgu |
| | S | 105 | TGACCTTCGCTGAGGCCCT |

Anti-TNR

| | | | |
|---|---|---|---|
| sirna_1715-g | A | 30 | NUAAUGACAGCGUAGACGNUG |
| | S | 44 | NCGUCUACGCUGUCAUUAN |
| sirna_1718-g | A | 31 | NUGCUAAUGACAGCGUAGNCG |
| | S | 45 | NCUACGCUGUCAUUAGCAN |
| sirna_1709-g | A | 32 | NCAGCGUAGACGCUGAUGNUG |
| | S | 46 | NCAUCAGCGUCUACGCUGN |
| sirna_4436-g | A | 33 | NUGGUCCGGUGGCAGUUCNUA |
| | S | 47 | NGAACUGCCACCGGACCAN |
| sirna_1809-g | A | 34 | NACGGUGGUCUCUGUGAUNGU |
| | S | 48 | NAUCACAGAGACCACCGUN |
| sirna_1710-g | A | 35 | NACAGCGUAGACGCUGAUNUU |
| | S | 49 | NAUCAGCGUCUACGCUGUN |
| sirna_1719-g | A | 36 | NUUGCUAAUGACAGCGUANAC |
| | S | 50 | NUACGCUGUCAUUAGCAAN |
| sirna_1716-g | A | 37 | NCUAAUGACAGCGUAGACNCU |
| | S | 51 | NGUCUACGCUGUCAUUAGN |
| sirna_1720-g | A | 38 | NGUUGCUAAUGACAGCGUNGA |
| | S | 52 | NACGCUGUCAUUAGCAACN |
| sirna_4495-g | A | 39 | NCCAAUGGUACCAGUUGANGC |
| | S | 53 | NUCAACUGGUACCAUUGGN |
| sirna_4432-g | A | 40 | NCCGGUGGCAGUUCUUAUNCC |
| | S | 54 | NAUAAGAACUGCCACCGGN |
| sirna_4431-g | A | 41 | NCGGUGGCAGUUCUUAUANCA |
| | S | 55 | NUAUAAGAACUGCCACCGN |
| sirna_1708-g | A | 42 | NAGCGUAGACGCUGAUGUNGU |
| | S | 56 | NACAUCAGCGUCUACGCUN |
| sirna_4494-g | A | 43 | NCAAUGGUACCAGUUGAUNCC |
| | S | 57 | NAUCAACUGGUACCAUUGN |
| sirna_1715 | A | 58 | CUAAUGACAGCGUAGACGCUG |
| | S | 72 | GCGUCUACGCUGUCAUUAG |
| sirna_1718 | A | 59 | UUGCUAAUGACAGCGUAGACG |
| | S | 73 | UCUACGCUGUCAUUAGCAA |
| sirna_1709 | A | 60 | ACAGCGUAGACGCUGAUGUUG |
| | S | 74 | ACAUCAGCGUCUACGCUGU |
| sirna_4436 | A | 61 | UUGGUCCGGUGGCAGUUCUUA |
| | S | 75 | AGAACUGCCACCGGACCAA |
| sirna_1809 | A | 62 | CACGGUGGUCUCUGUGAUCGU |
| | S | 76 | GAUCACAGAGACCACCGUG |
| sirna_1710 | A | 63 | GACAGCGUAGACGCUGAUGUU |
| | S | 77 | CAUCAGCGUCUACGCUGUC |
| sirna_1719 | A | 64 | GUUGCUAAUGACAGCGUAGAC |
| | S | 78 | CUACGCUGUCAUUAGCAAC |
| sirna_1716 | A | 65 | GCUAAUGACAGCGUAGACGCU |
| | S | 79 | CGUCUACGCUGUCAUUAGC |
| sirna_1720 | A | 66 | UGUUGCUAAUGACAGCGUAGA |
| | S | 80 | UACGCUGUCAUUAGCAACA |
| sirna_4495 | A | 67 | UCCAAUGGUACCAGUUGAUGC |
| | S | 81 | AUCAACUGGUACCAUUGGA |
| sirna_4432 | A | 68 | UCCGGUGGCAGUUCUUAUACC |
| | S | 82 | UAUAAGAACUGCCACCGGA |
| sirna_4431 | A | 69 | CCGGUGGCAGUUCUUAUACCA |
| | S | 83 | GUAUAAGAACUGCCACCGG |
| sirna_1708 | A | 70 | CAGCGUAGACGCUGAUGUUGU |
| | S | 84 | AACAUCAGCGUCUACGCUG |
| sirna_4494 | A | 71 | CCAAUGGUACCAGUUGAUGCC |
| | S | 85 | CAUCAACUGGUACCAUUGG |

N may be any nucleotide, wherein N at position 1 of the guide strand is complementary to N at position 19 of the corresponding sense strand, and N at position 19 of the guide strand is complementary to N at position 1 of the corresponding sense strand, the position is counted from the 5′ to the sequence. In the sequence listing file, T represents U in the sequences of RNA molecules.

The terms “a,” “an,” and “the” are used herein interchangeably and mean one or more. The term “and/or” is used to indicate one or both stated cases may occur, for example A and/or B includes. (A and B) and (A or B).

The term “or,” as used herein, denotes alternatives that may, where appropriate, be combined; that is, the term “or” includes each listed alternative separately as well as their combination if the combination is not mutually exclusive.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### Examples

### Example 1. Design of siRNA Inhibiting Neurocan Expression

An extended bioinformatic analysis was performed to find the best sequences that can be used for designing the siRNA to inhibit the expression of Neurocan (NCAN, the sequence of mRNA is in SEQ ID NO: 21). Sense and antisense (guide) sequences of the resulted siRNAs are presented in Table 2. In some cases, the sequence complementary to Guide (antisense) polynucleotides comprises from 14 to 19 nucleotides. siRNA to three particular regions on the sequence of NCAN gene provided the most prominent effect. The regions are nucleotides No. 1000-1300; 3700-4000, 500-800 and 1-150.

TABLE 2
Sequences used for the preparation of NCAN siRNA

| siRNA duplex | | SEQ | | | |
|---|---|---|---|---|---|
| ID | (A)/(S) | ID No | Sequence (5′-3′) | From | To |
| sirna_3816G | A | 11 | UACAGUGGCAUGGACAUUCUA | 3816 | 3834 |
| | S | 16 | GAAUGUCCAUGCCACUGUA | | |
| sirna_1172 | A | 12 | UGAGCUCGGAAGCAGUAGCCG | 1172 | 1190 |
| | S | 17 | GCUACUGCUUCCGAGCUCA | | |
| sirna_632 | A | 13 | UGGGCCUCAGCGAAGGUCCGU | 632 | 650 |
| | S | 18 | GGACCUUCGCUGAGGCCCA | | |
| sirna_1014 | A | 14 | UGGGUCGCACUGGUCCAGCCC | 1014 | 1032 |
| | S | 19 | GCUGGACCAGUGCGACCCA | | |
| sirna_55 | A | 15 | UCCGGGCACAAAGGACGCCGC | 55 | 73 |
| | S | 20 | GGCGUCCUUUGUGCCCGGA | | |
| sirna_3816GT | A | 86 | AACAGUGGCAUGGACAUUCua | 3816 | 3834 |
| | S | 96 | GAATGTCCATGCCACTGTT | | |
| sirna_3816T | A | 87 | AACAGUGGCAUGGACAUUGua | 3816 | 3834 |
| | S | 97 | CAATGTCCATGCCACTGTT | | |
| sirna_1172 | A | 88 | UGAGCUCGGAAGCAGUAGGcg | 1172 | 1190 |
| | S | 98 | CCTACTGCTTCCGAGCTCA | | |
| sirna_1172GT | A | 89 | AGAGCUCGGAAGCAGUAGCcg | 1172 | 1190 |
| | S | 99 | GCTACTGCTTCCGAGCTCT | | |
| sirna_1172T | A | 90 | AGAGCUCGGAAGCAGUAGGcg | 1172 | 1190 |
| | S | 100 | CCTACTGCTTCCGAGCTCT | | |
| sirna_632GT | A | 91 | AGGGCCUCAGCGAAGGUCCgu | 632 | 650 |
| | S | 101 | GGACCTTCGCTGAGGCCCT | | |
| sirna_632C | A | 92 | UGGGCCUCAGCGAAGGUCGgu | 632 | 650 |
| | S | 102 | CGACCTTCGCTGAGGCCCA | | |
| sirna_632CT | A | 93 | AGGGCCUCAGCGAAGGUCGgu | 632 | 650 |
| | S | 103 | CGACCTTCGCTGAGGCCCT | | |
| sirna_632 | A | 94 | UGGGCCUCAGCGAAGGUCAgu | 632 | 650 |
| | S | 104 | TGACCTTCGCTGAGGCCCA | | |
| sirna_632T | A | 95 | AGGGCCUCAGCGAAGGUCAgu | 632 | 650 |
| | S | 105 | TGACCTTCGCTGAGGCCCT | | |

S-sense; A-antisense; from/to refers to the position on the gene having a nucleic acid sequence SEQ ID NO: 21.

These sequences were chosen according to maximal homology between multiple species including human, mice, rats and rhesus (monkey) and their best inhibition probabilities after considering mismatches.

### Example 2. Design of siRNA Inhibiting Tenascin-R Expression

An extended bioinformatic analysis was performed to find the best sequences that can be used for designing the siRNA to inhibit the expression of Tenascin-R (TNR, the sequence of mRNA is in SEQ ID NO: 22). Sense and antisense (guide) sequences of the resulted siRNAs are presented in Table 3. In some cases, the sequence complementary to Guide (antisense) polynucleotides comprises from 14 to 19 nucleotides. siRNA to two particular regions on the sequence of TNR gene provided the most prominent effect. The regions are nucleotides No. 1600-2000 and 4300-4700.

TABLE 3
Sequences used for the preparation of TNR siRNA

| | Antisense | | | | |
|---|---|---|---|---|---|
| siRNA | (A)/ | SEQ-ID | | | |
| duplex ID | Sense (S) | No | Sequence (5′-3′) | From | to |
| sirna_1715 | A | 58 | CUAAUGACAGCGUAGACGCUG | 1715 | 1733 |
| | S | 72 | GCGUCUACGCUGUCAUUAG | | |
| sirna_1718 | A | 59 | UUGCUAAUGACAGCGUAGACG | 1718 | 1736 |
| | S | 73 | UCUACGCUGUCAUUAGCAA | | |
| sirna_1709 | A | 60 | ACAGCGUAGACGCUGAUGUUG | 1709 | 1727 |
| | S | 74 | ACAUCAGCGUCUACGCUGU | | |
| sirna_4436 | A | 61 | UUGGUCCGGUGGCAGUUCUUA | 4436 | 4454 |
| | S | 75 | AGAACUGCCACCGGACCAA | | |
| sirna_1809 | A | 62 | CACGGUGGUCUCUGUGAUCGU | 1809 | 1827 |
| | S | 76 | GAUCACAGAGACCACCGUG | | |
| sirna_1710 | A | 63 | GACAGCGUAGACGCUGAUGUU | 1710 | 1728 |
| | S | 77 | CAUCAGCGUCUACGCUGUC | | |
| sirna_1719 | A | 64 | GUUGCUAAUGACAGCGUAGAC | 1719 | 1737 |
| | S | 78 | CUACGCUGUCAUUAGCAAC | | |
| sirna_1716 | A | 65 | GCUAAUGACAGCGUAGACGCU | 1719 | 1737 |
| | S | 79 | CGUCUACGCUGUCAUUAGC | | |
| sirna_1720 | A | 66 | UGUUGCUAAUGACAGCGUAGA | 1720 | 1738 |
| | S | 80 | UACGCUGUCAUUAGCAACA | | |
| sirna_4495 | A | 67 | UCCAAUGGUACCAGUUGAUGC | 4495 | 4513 |
| | S | 81 | AUCAACUGGUACCAUUGGA | | |
| sirna_4432 | A | 68 | UCCGGUGGCAGUUCUUAUACC | 4432 | 4450 |
| | S | 82 | UAUAAGAACUGCCACCGGA | | |
| sirna_4431 | A | 69 | CCGGUGGCAGUUCUUAUACCA | 4431 | 4449 |
| | S | 83 | GUAUAAGAACUGCCACCGG | | |
| sirna_1708 | A | 70 | CAGCGUAGACGCUGAUGUUGU | 1708 | 1726 |
| | S | 84 | AACAUCAGCGUCUACGCUG | | |
| sirna_4494 | A | 71 | CCAAUGGUACCAGUUGAUGCC | 4494 | 4512 |
| | S | 85 | CAUCAACUGGUACCAUUGG | | |

S-sense; A-antisense; from/to refers to the position on the gene having a nucleic acid sequence SEQ ID NO: 22.

These sequences were chosen on their basis of the best homology between multiple species including human, mice, rats and rhesus (monkey) and their best inhibition probabilities after considering mismatches.

### Example 3

### Exosome Purification Protocol

Human MSCs were purchased from Lonza (Basel, Switzerland). Cells were cultured and expanded. Cells were cultured with exosome-free platelets lysate (Rabin Medical Center, Israel), and 3 days later, the medium was collected. The exosomes were purified using a standard differential centrifugation protocol, which involved isolating the culture fluid and centrifuging for 10 min at 300 g. The supernatant was recovered and centrifuged for 10 min at 2.000 g and then re-centrifuged for 30 min at 10,000 g. The supernatant was then passed through a 0.22 μm filter, and centrifuged for 70 min at 100,000 g. The pellet, containing the exosomes and proteins, was washed in PBS and then centrifuged for 70 min at 100,000 g. The pellet was re-suspended in 200 μl sterile PBS. All centrifugations were performed at 4° C. Exosomes were characterized using NanoSight technology, electron microscopy and Western blotting for calnexin, as a negative marker, and CD9 and CD81, as positive marker.

Loading of NCAN OR TNR siRNA to MSC-Exo

The siRNA, e.g., those obtained in Examples 1 and 2, are conjugated to cholesterol-TEG in the 3′ of the sense (guide) strand. Cholesterol is used as a loading reagent/enhancer of the siRNA into the exosomes. The loading protocol is performed by co-incubation of the cholesterol-teg-siRNA molecules (1 μl of 100 μM) with 40 μl of 10⁶-10⁸ exosomes per μl at 37° C. for 2-4 hours.

After the incubation, the free cholesterol-teg-siRNA is washed, e.g. by 30 kDa Amicon and/or ultracentrifugation of 100G for 2-4 hours. The pellet, containing the loaded exosomes is used for in-vivo and in-vitro experiments.

To quantify the amount of siRNA in the loaded exosomes, the fluorescent marker (cy3 for example) is conjugated to the 5′ of the sense strand, and the fluorescent signal is compared to the calibration curve.

Specifically, EVs were loaded with the following anti-NCAN siRNAs: sirna_3816G (comprising oligonucleotides having sequences UACAGUGGCAUGGACAUUCUA and GAAUGUCCAUGCCACUGUA), sirna_1172GA (comprising oligonucleotides having sequences UGAGCUCGGAAGCAGUAGCCG and GCUACUGCUUCCGAGCUCA) and sirna_632GA (comprising oligonucleotides having sequences UGGGCCUCAGCGAAGGUCCGU and GGACCUUCGCUGAGGCCCA); and anti-TNR siRNAs: sirna_1715 (comprising oligonucleotides having sequences CUAAUGACAGCGUAGACGCUG and GCGUCUACGCUGUCAUUAG), sirna_4436 (comprising oligonucleotides having sequences UUGGUCCGGUGGCAGUUCUUA and AGAACUGCCACCGGACCAA) and sirna_1809 (comprising oligonucleotides having sequences CACGGUGGUCUCUGUGAUCGU and GAUCACAGAGACCACCGUG).

An additional analytic method to determine the loading efficacy is HPLC which allows detecting the siRNA in the exosomes by its molecular characteristic and provide an analytical measurement of amounts.

### Example 4

The efficacy of several sequences with the best SVM scores, and their ability to decrease TNR gene expression was tested on SK—N-SH cells (neuroblastoma cell line). The results were obtained after transfection of the cells using Lipofectamine reagent and 20 nM siRNA molecules against TNR for 24 hours. From these results, we chose the most potent duplexes for further studies. The tested antiTNR siRNAs are presented in Table 4. Some of the siRNA were conjugated with cholesterol (chol), cy3 or FAM, as described in the Table. In all siRNA the guide oligonucleotide comprises 21 nt and the sense oligonucleotide 19 nt except for TNR6 comprising 20 and 15 nt, respectively. This first screening revealed the most effective molecules.

TABLE 4
Anti-TNR siRNA (duplexes)used in the experiments

| Name | Sequence from report | 3′ end | 5′ end |
|---|---|---|---|
| TNR 1 | TNR_1715 (SEQ ID NOs: 58 and 72) | | |
| TNR 2 | TNR_1715 | chol | |
| TNR 3 | TNR_1715 | | cy3 |
| TNR 4 | TNR_1715 | chol | cy3 |
| TNR 5 | TNR_1715 | chol | FAM |
| TNR 6* | TNR_1715 | | |
| TNR 7 | TNR_1809 (SEQ ID NOs: 62 and 76) | | |
| TNR 8 | TNR_1809 | chol | |
| TNR 9 | TNR_4436 (SEQ ID NOs: 61 and 75) | | |
| TNR 10 | TNR_4436 | chol | |

TNR6* - shorter siRNA

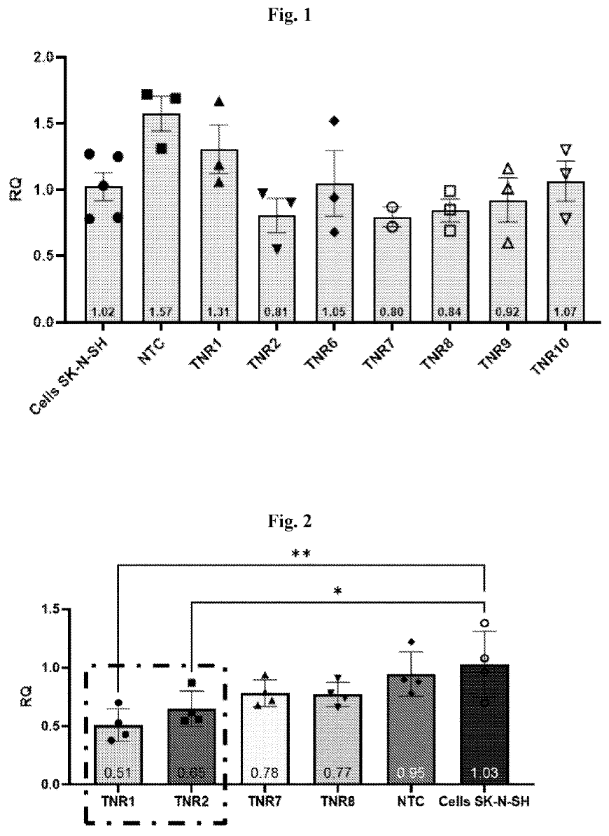

The effect of different anti-TNR siRNAs (20 nM) on the expression of TNR in SK—N-SH cells transfected with using Lipofectamine™ 3000 reagent is presented in FIG. 1. Relative expression was evaluated with TaqMan probes by qRT-PCR. Expression of GAPDH gene was used as an internal control.

As follows from the results, TNR2 (sirna_1715, sequences CUAAUGACAGCGUAGACGCUG and GCGUCUACGCUGUCAUUAG) and TNR7 (sirna_1809, sequences CACGGUGGUCUCUGUGAUCGU and GAUCACAGAGACCACCGUG) showed the best results. These siRNAs aim at two different sites on the TnR gene. TNR2 is cholesterol conjugated and TNR7 is not conjugated.

To find the optimal conditions for analysis, we pursued with sirna_1715 and sirna_1809 siRNAs, either conjugated with cholesterol or not; sirna_1715-TNR 1 and TNR2 (with cholesterol) and sirna_1809-TNR7 and TNR8 (with cholesterol). Different time points and a different siRNA concentration were tested to find the most effective siRNA duplex and the optimal conditions of the transfection.

The results of 24-hour transfection are presented in FIG. 2. Relative expression was evaluated with TaqMan probes by qRT-PCR. Expression of GAPDH gene was used as an internal control. It can be seen from these results that TNR1 and TNR2 (sirna_1715) provided the best results with the reduction in expression of TNR by 40-50%. The results are statistically significant. These siRNAs are further investigated to determine their ability to reduce TnR expression in cells when loaded into EVs.

Protein expression of TNR following transfection and following the addition of siTNR-loaded EVs are assessed using western blot or ELISA (of lysates and medium) using an antibody specific to TNR.

### Example 5

The efficacy of several sequences with the best SVM scores, and their ability to decrease NCAN gene expression was tested on SK—N-SH cells. We first evaluated the efficacy of NCAN siRNA novel duplexes on decreasing NCAN-RNA levels as follows: SK—N-SH cells were transfected with si-NCAN duplexes (20 nM) using Lipofectamine™ 3000 reagent for 24 hours. Relative expression was evaluated with TaqMan probes by qRT-PCR. Expression of GAPDH gene was used as an internal control. Then, the most potent duplexes were chosen for further studies. The tested anti NCAN siRNAs are presented in Table 5. Some of the siRNA sequences were conjugated with cholesterol (chol), cy3 or FAM, as described in the Table.

TABLE 5
anti-NCAN siRNA (duplexes)used in the experiments

| Duplex | | | |
|---|---|---|---|
| # | Sequence from report | 3′ end | 5′ end |
| NCAN 1 | NCAN_3816 (SEQ ID NOs: 11 and | — | — |
| | 16) | | |
| NCAN 2 | NCAN_3816 | Cholesterol | — |
| NCAN 3 | NCAN_3816 | — | cy3 |
| NCAN 4 | NCAN_3816 | Cholesterol | cy3 |
| NCAN 5 | NCAN_1172 (SEQ ID NOs: 12 and | — | — |
| | 17) | | |
| NCAN 6 | NCAN_1172 | Cholesterol | — |
| NCAN 7 | NCAN_632 (SEQ ID NO: 13 and | — | — |
| | 18) | | |
| NCAN 8 | NCAN_632 | Cholesterol | — |

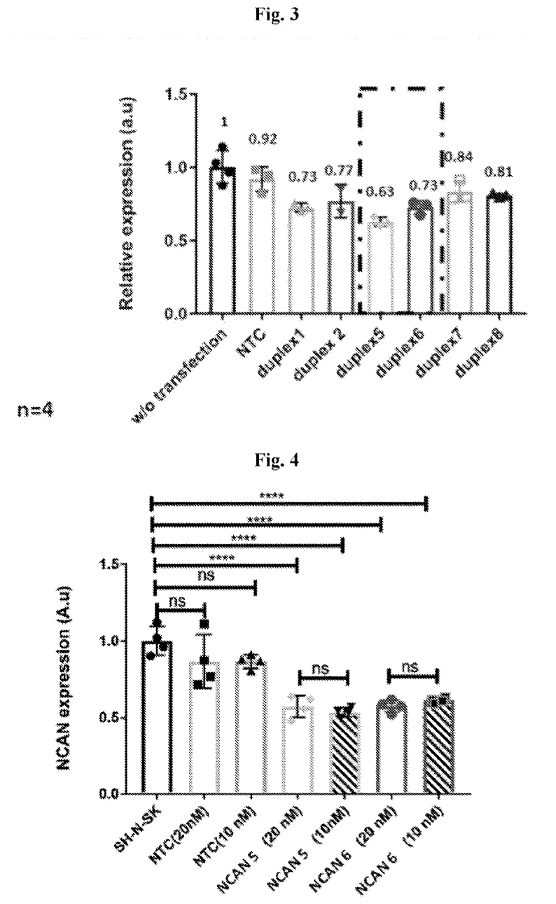

The results are presented in FIG. 3. NCAN1 siRNA is denoted in the Figure as duplex 1, NCAN2 as duplex 2 and so on. It can be seen that siRNA sirna_1172 either (sequences UGAGCUCGGAAGCAGUAGCCG and GCUACUGCUUCCGAGCUCA) without or with cholesterol (NCAN5 and NCAN6, respectively) provided the best results.

To find the optimal conditions for analysis, we checked another time point and a different siRNA concentration (10 nM). We found that at 48 hours post-transfection RNA expression levels were decreased more than at 24 hours post-transfection and that NCAN relative expression was not significantly different after transfection with 10 nM compared to 20 nM (FIG. 4). Relative expression was evaluated with TaqMan probes by qRT-PCR. Expression of GAPDH gene was used as an internal control.

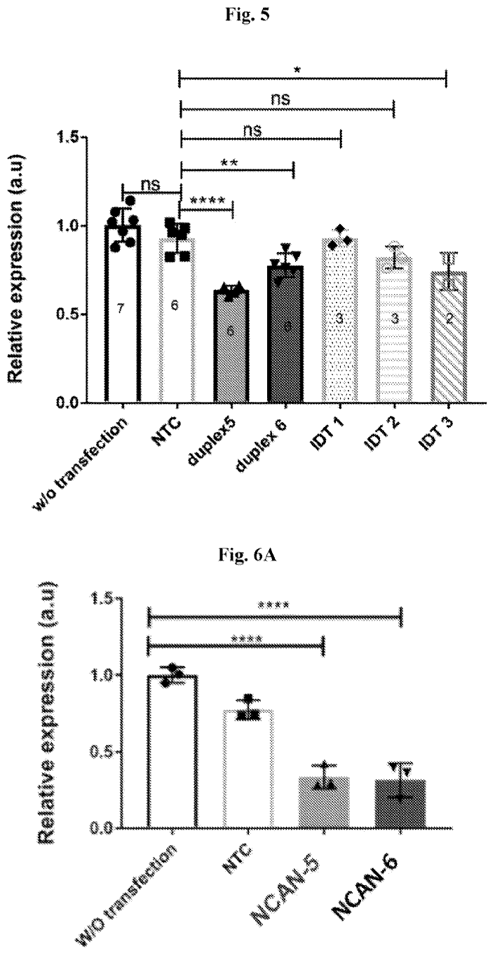

Further, the efficacy of NCAN5 and NCAN6 was compared with the efficacy of commercially available anti-NCAN siRNAs purchased from IDT (Integrated DNA Technologies) and denoted as IDT1, IDT2 and IDT3. SK—N-SH cells were transfected with 20 nM anti-NCAN siRNA duplexes (NurExone or IDT) using Lipofectamine™ 3000 reagent with reverse transfection. Relative expression was evaluated with TaqMan probes by qRT-PCR 48 hr post-transfection. Expression of GAPDH gene was used as an internal control. As follows from FIG. 5, NCAN5 and NCAN6 have higher efficacy than the commercial siRNAs.

### Example 6

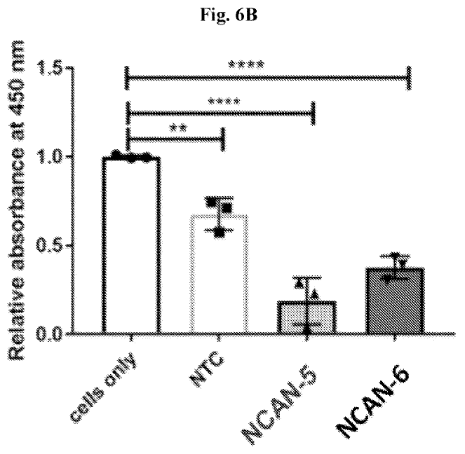

Since NCAN is a secreted chondroitin sulfate proteoglycan, the effect of different anti-NCAN siRNA duplexes on secretion of NCAN protein in the media was assessed. SK—N-SH cells were transfected with si-NCAN duplexes (20 nM) using Lipofectamine™ 3000 reagent. 48 hr post-transfection RNA was isolated and the media was collected. Protein levels were evaluated with NCAN ELISA kit. Relative expression was evaluated with TaqMan probes by qRT-PCR. The results are presented in FIGS. 6A and 6B. It can be seen that gene expression was reduced by NCAN5 and NCAN6 by more than 50% and protein expression was reduced by NCAN5 by about 80% and by NCAN6 by about 60%

### Example 7

ReN cells VM (cat #SCC008, Millipore) are cultured at a seeding density of 5×10⁴ cells/cm², with regular medium changes every 48 hours. The ReN cells proliferation medium consists of ReNcell NSC Maintenance Medium (cat #SCM005, Sigma) supplemented with 20 ng/ml hEGF (cat #GF001, Merck) and hbFGF (cat #GF003, Merck). Once the cells reach a monolayer, about 4 days post-seeding, the proliferation medium is replaced with the differentiation medium, which consists of the maintenance medium without supplements. Cells are continuously monitored. The medium is replaced every 48 hours. After 2 weeks, as per the manufacturer's instructions, the cells become completely differentiated into adult neurons, accompanied by the formation of PNN (Perineuronal Nets).

To confirm the successful differentiation into neural cells, Wisteria floribunda agglutinin (WFA) (ca #FL-1351-2, Vector laboratories) staining of the PNN as per the manufacturer's instructions and NeuN (1:500) (Ab177487, Abcam) labeling of the neurons is performed.

Furthermore, the level of neural markers' enrichment is evaluated using RT-qPCR with the NeuN Taqman Assay (cat #AB431182, Rhenium). The assessment of enrichment is calculated in comparison to non-differentiated ReN cells according to relative quantification calculations (AACT).

Following this, the cells are treated with EVs (2×10⁸ particles/cm²) as previously optimized for 48 hours. The treatments will include Naïve EVs, anti-NCAN-siRNA-loaded EVs, or anti-NCAN-siRNA-loaded EVs. The used anti-NCAN-siRNA are sirna_3816G (SEQ ID NOs: 11 and 16), sirna_1172GA (SEQ ID NOs: 12 and 17) and sirna_632GA (SEQ ID NOs: 13 and 18). The used anti-TNR-siRNA are sirna_1715 (SEQ ID NOs: 58 and 72), sirna_4436 (SEQ ID NOs: 51 and 75) and sirna_1809 (SEQ ID NOs: 62 and 76). Subsequently, RNA isolation is carried out with QIAGEN RNeasy Mini Kit (cat #74104), and the levels of NCAN and TNR RNA is assessed using RT-qPCR. Additionally, fluorescent staining using WFA antibodies is performed to visualize any decrease in PNN formation as per the manufacturer's instructions. It should be noted that the protocol may be further optimized.

Additionally, a similar assay will be performed on differentiated REN cells following a scratch injury of the cells, and sprouting and proliferation of the neuronal cells will be observed and quantified by confocal microscopy in order to determine the effect of the treatment on neural regeneration.

### Example 8

The PNN structural integrity may be measured by histological staining of the spinal cord and brain, by labeling on or more of its structural proteins, e.g. as described in Christensen et al., Nat Commun. 2021; 12 (1): 1-17. doi: 10.1038/s41467-020-20241-w or Lensjø et al., J Neurosci. 2017; 37 (5): 1269-1283. doi: 10.1523/JNEUROSCI.2504-16.2016). For this procedure, rats or mice are treated with 20 μl intranasal administration of siRNA of the chosen target as is, and loaded to exosomes (10⁷-10⁸ exosomes/μl). In some examples, the mice or rats are treated with exosomes loaded with siRNA inhibiting the production of NCAN. The exosomes are, e.g., as described in Example 3. Specifically, exosomes comprising siRNA as described in Table 1 and in Table 2 are used. After the treatment, the animals are sacrificed and the brain/spinal cord is fixated using 5% Paraformaldehyde (PFA) and the relevant structural proteins of the PNN are labeled using primary and secondary antibodies. Comparison analysis is performed between the structure of the PNN from treated vs untreaded animals to measure the inhibitory effect of the siRNA on the PNN formation/integrity/disintegration.

Another efficacy experiment is done by in-vivo for testing the efficacy of the treatment as described above after complete transection and/or impaction of the spinal cord. The motor functions are examined. The efficacy of the treatment is being measured by motor improvement of a scale named BBB score and also sensory recovery.

### Example 9. In vivo efficacy of anti-NCAN and anti-TNR siRNAs

Rats are operated on for a complete transection of the spinal cord a T10 and divided into four treatments groups:
1. Exosomes loaded with anti-NCAN siRNA sirna_3816G (sequences UACAGUGGCAUGGACAUUCUA and GAAUGUCCAUGCCACUGUA);
2. Exosomes loaded with anti-NCAN siRNA sirna_1172GA (sequences UGAGCUCGGAAGCAGUAGCCG and GCUACUGCUUCCGAGCUCA);
3. Exosomes anti-NCAN siRNA sirna_632GA (sequences UGGGCCUCAGCGAAGGUCCGU and GGACCUUCGCUGAGGCCCA);
4. Exosomes loaded with anti-TNR siRNA sirna_1715 sequences CUAAUGACAGCGUAGACGCUG and GCGUCUACGCUGUCAUUAG)
5. Exosomes loaded with anti-TNR siRNA sirna_4436 (sequences UUGGUCCGGUGGCAGUUCUUA and AGAACUGCCACCGGACCAA)
6. Exosomes loaded with anti-TNR siRNA sirna_1809 (comprising oligonucleotides having sequences CACGGUGGUCUCUGUGAUCGU and GAUCACAGAGACCACCGUG).
7. siRNA sirna_3816G
8. siRNA sirna_1172GA
9. siRNA sirna_632GA
10. siRNA sirna_1715
11. siRNA sirna_4436
12. Exosomes only (n=4)
13. Saline (n=6)

Rats receive treatment for 5 days starting on the day of surgery, have 2 days break, and then receive treatment for another 5 days.

From 1 week after the surgery, rats are exercised on a treadmill 5 days a week and were tested for Dorsal Von Frey, weighed, and recorded walking for BBB scoring weekly. During the 10th week, rats' spinal cords were scanned by MRI.

Reflexes recovery is tested by tail and paw pinch 2 weeks after surgery.

The well-being of rats is evaluated by the self-eating tendency of treated rats following the injury. Rats operated on for SCI are prone to self-eating.

Sagittal MRI images, axial sections, and cross-sectional area images 4 mm caudal and rostral to the T10 epicenter in healthy rats or to the injury epicenter in untreated, and treated rats are analyzed and the ratio of caudal to rostral area is calculated, representing the regeneration of the tissue downstream the injury.

### Example 10. Loading of NCAN OR TNR siRNA to MSC-exo

The siRNA, e.g., those obtained in Examples 1 and 2, are conjugated to glucose or sucrose in the 3′ of the sense (guide) strand. Glucose or sucrose is used as a loading reagent/enhancer of the siRNA into the exosomes. The loading protocol is performed by co-incubation of the glucose-siRNA molecules (1 μl of 100 μM) with 40 μl of 10⁶-10⁸ exosomes per μl at 37° C. for 2-4 hours.

After the incubation, the free glucose-siRNA or sucrose-siRNA is washed, e.g. by 30 kDa Amicon and/or ultracentrifugation of 100G for 2-4 hours. The pellet, containing the loaded exosomes is used for in-vivo and in-vitro experiments.

To quantify the amount of siRNA in the loaded exosomes, the fluorescent marker (cy3 for example) is conjugated to the 5′ of the sense strand, and the fluorescent signal is compared to the calibration curve.

Specifically, EVs are loaded with the following anti-NCAN siRNAs conjugated with glucose: sirna_3816G (comprising oligonucleotides having sequences UACAGUGGCAUGGACAUUCUA and GAAUGUCCAUGCCACUGUA), sirna_1172GA (comprising oligonucleotides having sequences UGAGCUCGGAAGCAGUAGCCG and GCUACUGCUUCCGAGCUCA) and sirna_632GA (comprising oligonucleotides having sequences UGGGCCUCAGCGAAGGUCCGU and GGACCUUCGCUGAGGCCCA); and anti-TNR siRNAs: sirna_1715 (comprising oligonucleotides having sequences CUAAUGACAGCGUAGACGCUG and GCGUCUACGCUGUCAUUAG), sirna_4436 (comprising oligonucleotides having sequences UUGGUCCGGUGGCAGUUCUUA and AGAACUGCCACCGGACCAA) and sirna_1809 (comprising oligonucleotides having sequences CACGGUGGUCUCUGUGAUCGU and GAUCACAGAGACCACCGUG).

Although the present invention has been described herein above by way of preferred embodiments thereof, it can be modified, without departing from the spirit and nature of the subject invention as defined in the appended claims.

1. An RNA interference (RNAi) oligonucleotide selected from siRNA and shRNA comprising a guide strand comprising a nucleic acid sequence selected from SEQ ID NO: 1-5 and 30-43 for inhibiting expression of a protein of a perineuronal network selected from Neurocan (NCAN) and Tenascin-R (TNR).

2. (canceled)

3. The RNAi oligonucleotide according to claim 1, wherein RNAi oligonucleotide inhibits expression of (i) Neurocan protein and the guide strand comprises a nucleic acid sequence selected from SEQ ID NO: 1-5, 11-15 and 86-95; or (ii) Tenascin-R (TNR) and the guide strand comprises a nucleic acid sequence selected from SEQ ID NO: 30-43 and 58-71.

4. (canceled)

5. The RNAi oligonucleotide according to claim 3, wherein (i) the RNAi is siRNA and wherein the guide strand consists of a nucleic acid sequence selected from SEQ ID NO: 1-5, 11-15, and 86-95 or (ii) wherein the RNAi is siRNA and wherein the guide strand consists of a nucleic acid sequence selected from SEQ ID NO: 30-43 and 58-71.

6. The RNAi oligonucleotide according to claim 3, comprising a strand complementary to said guide strand, wherein the complementary strand is complementary to at least 14, 15, 16, 17, 18, 19, 20 or 21 contiguous nucleotides of the guide strand.

7. The RNAi oligonucleotide according to claim 6, wherein (i) the RNAi oligonucleotide inhibits expression of Neurocan protein and the complementary strand comprises a nucleic acid sequence selected from SEQ ID NOs: 6-10, 16-20 and 96-105, or (ii) the RNAi oligonucleotide inhibits expression of TNR and the complementary strand comprises a nucleic acid sequence selected from SEQ ID NO: 44-57 and 72-85, and wherein the complementary strand comprises at positions 1 and 19 nucleic acids that are complementary to the nucleic acids at the corresponding positions in the sequence of said guide strand.

8. (canceled)

9. The RNAi oligonucleotide according to claim 37, wherein
(i) the RNAi oligonucleotide is siRNA comprising a guide strand and a complementary strand, wherein the guide strand comprises a nucleic acid sequence selected from SEQ ID NO: 11-15 and 86-95 and the complementary strand comprises a nucleic acid sequence selected from SEQ ID NO: 16-20 and 96-105 or (ii) wherein the RNAi is siRNA comprising a guide strand and a complementary strand, wherein the guide strand comprises a nucleic acid sequence selected from SEQ ID NO: 44-57 and the complementary strand comprises a nucleic acid sequence selected from SEQ ID NOs: 72-85.

10. The RNAi oligonucleotide according to claim 7, wherein (i) the RNAi is siRNA inhibiting the expression of Neurocan protein and comprising a pair of a guide strand and a complementary strand, wherein the pair comprises or consists of nucleic acid sequences SEQ ID NOs: 11 and 16; SEQ ID NOs: 12 and 17; SEQ ID NO: 13 and 18, SEQ ID NOs: 86 and 96, SEQ ID NOs: 87 and 77, SEQ ID NO: 88 and 88, SEQ ID NOs: 89 and 99, SEQ ID NOs: 90 and 100, SEQ ID NOs: 91 and 101, SEQ ID NOs: 92 and 102, SEQ ID NOS: 93 and 103, SEQ ID NOs: 94 and 104, or SEQ ID NOs: 95 and 105, or
(ii) the RNAi the RNAi is siRNA inhibiting the expression of TNR and comprising a pair of a guide strand and a complementary strand, wherein the pair comprises or consists of nucleic acid sequences SEQ ID NOs: 58 and 72; SEQ ID NOs: 61; and 75 or SEQ ID NO: 62 and 76.

11. (canceled)

12. (canceled)

13. (canceled)

14. (canceled)

15. (canceled)

16. (canceled)

17. (canceled)

18. (canceled)

19. (canceled)

20. The RNAi oligonucleotide according to claim 1, conjugated with a hydrophobic molecule or a carbohydrate.

21. The RNAi oligonucleotide according to claim 20, wherein said hydrophobic moiety is selected from the group consisting of a sterol, a ganglioside, a lipid, a vitamin, a fatty acid, a hydrophobic peptide, and a combination thereof, optionally said hydrophobic moiety is cholesterol and said carbohydrate is selected from glucose and sucrose.

22. (canceled)

23. (canceled)

24. (canceled)

25. Isolated extracellular vesicles (EVs) comprising RNA interference (RNAi) oligonucleotides selected from siRNA and shRNA inhibiting expression of a protein of a perineuronal network.

26. The isolated EVs according to claim 25, characterized by at least one of:
(i) the protein is selected from Neurocan (NCAN), Tenascin-R, Aggrecan (ACAN), Versican (VCAN), Brevican (BCAN) and any combination thereof;
(ii) the EVs are selected from exosomes, microvesicles, and a combination thereof;
(iii) the extracellular vesicles are exosomes
(iv) said extracellular vesicles are derived from adherent cells expressing mesenchymal markers;
(v) said extracellular vesicles are derived from adherent cells expressing mesenchymal markers selected from mesenchymal stem cells (MSC) and olfactory ensheathing cells;
(vi) the EVs further comprising a compound selected from chondroitinase ABC, matrix metalloproteinases (MMPs), a disintegrin metalloproteinase with thrombospondin motifs (ADAMTS), a nucleic acid molecule encoding thereof and hyaluronic acid (HA).

27. (canceled)

28. (canceled)

29. (canceled)

30. (canceled)

31. The isolated EVs according to claim 25, wherein the EVs comprise (i) RNAi oligonucleotides inhibiting expression of NCAN protein, (ii) RNAi oligonucleotides inhibiting expression of TNR, of (iii) both (i) and (ii), wherein: RNAi oligonucleotide inhibiting expression of NCAN protein comprises a guide strand comprising a nucleic acid sequence selected from SEQ ID NO: 1-5, 11-15 and 86-95; and the RNAi oligonucleotide inhibiting expression of Tenascin-R (TNR) comprise a guide strand comprising a nucleic acid sequence selected from SEQ ID NO: 30-43 and 58-71.

32. (canceled)

33. The isolated EVs according to claim 31, wherein the RNAi oligonucleotide inhibiting expression of NCAN protein is siRNA comprising a pair of a guide strand and a complementary strand, wherein the pair comprises or consists of nucleic acid sequences SEQ ID NOs: 11 and 16, SEQ ID NOs: 12 and 17, SEQ ID NO: 13 and 18, SEQ ID NOs: 86 and 96, SEQ ID NOs: 87 and 77, SEQ ID NO: 88 and 88, SEQ ID NOs: 89 and 99, SEQ ID NOs: 90 and 100, SEQ ID NOs: 91 and 101, SEQ ID NOs: 92 and 102, SEQ ID NOs: 93 and 103, SEQ ID NOs: 94 and 104, or SEQ ID NOs: 95 and 105 and RNAi oligonucleotide inhibiting expression of TNR sequences is siRNA comprising a pair of a guide strand and a complementary strand SEQ ID NOs: 58 and 72, SEQ ID NOs: 61 and 75, or SEQ ID NO: 62 and 76.

34. (canceled)

35. (canceled)

36. (canceled)

37. (canceled)

38. (canceled)

39. (canceled)

40. (canceled)

41. (canceled)

42. (canceled)

43. (canceled)

44. A pharmaceutical composition comprising RNAi oligonucleotides according to claim 1 or isolated EVs comprising said RNAi oligonucleotides, and a pharmaceutically acceptable excipient.

45. The pharmaceutical composition according to claim 44, formulated for administration via an administration route selected from intranasal, intra-lesion, intrathecal, intravenous, intramuscular, subcutaneous, sublingual, oral, and intracerebral administration route, optionally wherein the composition further comprising a compound selected from chondroitinase ABC, HA, matrix metalloproteinases (MMPs), a disintegrin and metalloproteinase with thrombospondin motifs (ADAMTS).

46. (canceled)

47. (canceled)

48. (canceled)

49. (canceled)

50. (canceled)

51. A method of inducing neuroregeneration or treating a neuronal injury or damage in a subject in need thereof, comprising administering to the subject a therapeutically effective amount of isolated extracellular vesicles comprising RNAi oligonucleotides inhibiting the expression of a protein selected from Neurocan (NCAN), Tenascin-R, Aggrecan (ACAN), Versican (VCAN), Brevican (BCAN) and a combination thereof, wherein RNAi oligonucleotides are according to claim 1.

52. (canceled)

53. The method according to claim 51, wherein the administering is comprises intranasal or injectable administration.

54. The method according to claim 51, further comprising administering a compound selected from chondroitinase ABC matrix metalloproteinases (MMPs), a disintegrin metalloproteinase with thrombospondin motifs (ADAMTS), a nucleic acid molecule encoding thereof and hyaluronic acid (HA).


SEQUENCE LISTING (105 sequences)

SEQ ID NO: 1 - RNA, 21 nt, synthetic construct
  source 1..21: mol_type = other RNA
nacagtggcatggacattnta

SEQ ID NO: 2 - RNA, 21 nt, synthetic construct
  source 1..21: mol_type = other RNA
ngagctcggaagcagtagncg

SEQ ID NO: 3 - RNA, 21 nt, synthetic construct
  source 1..21: mol_type = other RNA
ngggcctcagcgaaggtcngt

SEQ ID NO: 4 - RNA, 21 nt, synthetic construct
  source 1..21: mol_type = other RNA
ngggtcgcactggtccagncc

SEQ ID NO: 5 - RNA, 21 nt, synthetic construct
  source 1..21: mol_type = other RNA
nccgggcacaaaggacgcngc

SEQ ID NO: 6 - RNA, 19 nt, synthetic construct
  source 1..19: mol_type = other RNA
naatgtccatgccactgtn

SEQ ID NO: 7 - RNA, 19 nt, synthetic construct
  source 1..19: mol_type = other RNA
nctactgcttccgagctcn

SEQ ID NO: 8 - RNA, 19 nt, synthetic construct
  source 1..19: mol_type = other RNA
ngaccttcgctgaggcccn

SEQ ID NO: 9 - RNA, 19 nt, synthetic construct
  source 1..19: mol_type = other RNA
nctggaccagtgcgacccn

SEQ ID NO: 10 - RNA, 19 nt, synthetic construct
  source 1..19: mol_type = other RNA
ngcgtcctttgtgcccggn

SEQ ID NO: 11 - RNA, 21 nt, synthetic construct
  source 1..21: mol_type = other RNA
tacagtggcatggacattcta

SEQ ID NO: 12 - RNA, 21 nt, synthetic construct
  source 1..21: mol_type = other RNA
tgagctcggaagcagtagccg

SEQ ID NO: 13 - RNA, 21 nt, synthetic construct
  source 1..21: mol_type = other RNA
tgggcctcagcgaaggtccgt

SEQ ID NO: 14 - RNA, 21 nt, synthetic construct
  source 1..21: mol_type = other RNA
tgggtcgcactggtccagccc

SEQ ID NO: 15 - RNA, 21 nt, synthetic construct
  source 1..21: mol_type = other RNA
tccgggcacaaaggacgccgc

SEQ ID NO: 16 - RNA, 19 nt, synthetic construct
  source 1..19: mol_type = other RNA
gaatgtccatgccactgta

SEQ ID NO: 17 - RNA, 19 nt, synthetic construct
  source 1..19: mol_type = other RNA
gctactgcttccgagctca

SEQ ID NO: 18 - RNA, 19 nt, synthetic construct
  source 1..19: mol_type = other RNA
ggaccttcgctgaggccca

SEQ ID NO: 19 - RNA, 19 nt, synthetic construct
  source 1..19: mol_type = other RNA
gctggaccagtgcgaccca

SEQ ID NO: 20 - RNA, 19 nt, synthetic construct
  source 1..19: mol_type = other RNA
ggcgtcctttgtgcccgga

SEQ ID NO: 21 - RNA, 6402 nt, Homo sapiens
  source 1..6402: mol_type = mRNA
ggagcgcagggcgcaggggctggacccggcgcggagctggctgagtcggagcgcagcgtcctttgtgcccggcggccgccccgggatgcgtccgagctaggagccagatccaggatgggggccccgtttgtctgggccttgggccttttgatgctgcagatgctgctctttgtggctggggaacagggcacacaggatatcaccgatgccagcgaaagggggctccacatgcagaagctggggtctgggtcagtgcaggctgcgctggcggagctggtggccctgccctgtctctttaccctgcagccacggccaagcgcagcccgagatgcccctcggataaagtggaccaaggtgcggactgcgtcgggccagcgacaggacttgcccatcctggtggccaaggacaatgtcgtgagggtggccaaaagctggcagggacgagtgtcactgccttcctacccccggcgccgagccaacgccacgctacttctggggccactgagggccagtgactctgggctgtaccgctgccaggtggtgaggggcatcgaggatgagcaggacctggtgcccttggaggtgacaggtgttgtgttccactaccgatcagcccgggaccgctatgcactgaccttcgctgaggcccaggaggcctgccgtctcagctcagccatcattgcagcccctcggcatctacaggctgcctttgaggatggctttgacaactgtgatgctggctggctctctgaccgcactgttcggtatcctatcacccagtcccgtcctggttgctatggcgaccgtagcagccttccaggggttcggagctatgggaggcgcaacccacaggaactctacgatgtgtattgctttgcccgggagctggggggcgaggtcttctacgtgggcccggcccgccgcctgacactggccggcgcgcgtgcacagtgccgccgccagggtgccgcgctggcctcggtgggacagctgcacctggcctggcatgagggcctggaccagtgcgacccgggctggctggccgacggcagcgtgcgctacccgatccagacgccgcgccggcgctgcgggggcccagccccgggcgtgcgcaccgtctaccgcttcgctaaccggaccggcttcccctcacccgccgagcgcttcgacgcctactgcttccgagctcatcaccccacgtcacaacatggagacctagagaccccatcctctggggatgagggggagattctgtcagcagaggggcccccagttagagaactggagcccaccctggaggaggaagaggtggtcacccctgacttccaggagcctctggtgtccagtggggaagaagaaaccctgattttggaggagaagcaggagtctcaacagaccctcagccctacccctggggaccccatgctggcctcatggcccactggggaagtgtggctaagcacggtggcccccagccctagcgacatgggggcaggcactgcagcaagttcacacacggaggtggccccaactgaccctatgcctaggagaagggggcgcttcaaagggttgaatgggcgctacttccagcagcaggaaccggagccggggctgcaaggggggatggaggccagcgcccagccccccacctcagaggctgcagtgaaccaaatggagcctccgttggccatggcagtcacagagatgttgggcagtggccagagccggagcccctgggctgatctgaccaatgaggtggatatgcctggagctggttctgctggtggcaagagctccccagagccctggctgtggccccctaccatggtcccacccagcatctcaggccacagcagggcccctgtcctggagctagagaaagccgagggccccagtgccaggccagccaccccagacctgttttggtcccccttggaggccactgtctcagctcccagccctgccccctgggaggcattccctgtggccacctccccagatctccctatgatggccatgctgcgtggtcccaaagagtggatgctaccacaccccacccccatctccaccgaggccaatagagttgaggcacatggtgaggccaccgccacggctccaccctcccctgctgcagagaccaaggtgtattccctgcctctctctttgaccccaacaggacagggtggagaggccatgcccacaacacctgagtcccccagggcagacttcagagaaactggggagaccagccctgctcaggtcaacaaagctgagcactccagctccagcccatggccttctgtaaacaggaatgtggctgtaggttttgtccccactgagactgccactgagccaacgggcctcaggggtatcccggggtctgagtctggggtcttcgacacagcagaaagccccacttctggcttgcaggccactgtagatgaggtgcaggacccctggccctcagtgtacagcaaagggctggatgcaagttccccatctgcccccctggggagccctggagtcttcttggtacccaaagtcaccccaaatttggagccttgggttgctacagatgaaggacccactgtgaatcccatggattccacagtcacgccggcccccagtgatgctagtggaatttgggaacctggatcccaggtgtttgaagaagccgaaagcaccaccttgagccctcaggtggccctggatacaagcattgtgacgcccctcacgaccctggagcagggggacaaggttggagttccagccatgtctacactgggctcctcaagctcccaaccccacccagagccagaggatcaggtggagacccagggaacatcaggagcttcagtgcctccgcatcagagcagtcccctagggaaaccggctgttcctcctgggacaccgactgcagccagtgtgggcgagtctgcctcagtttcctcaggggagcctacggtaccgtgggacccctccagcaccctgctgcctgtcaccctgggcatagaggacttcgaactggaggtcctggcagggagcccgggtgtagagagcttctgggaggaggtggcaagtggagaggagccagccctgccagggacccctatgaatgcaggtgcggaggaggtgcactcagatccctgtgagaacaacccttgtcttcatggagggacatgtaatgccaatggcaccatgtatggctgtagctgtgatcagggcttcgccggggagaactgtgagattgacattgatgactgcctctgcagcccctgtgagaatggaggcacctgtattgatgaggtcaatggctttgtctgcctttgcctccccagctatgggggcagcttttgtgagaaagacaccgagggctgtgaccgcggctggcataagttccagggccactgttaccgctattttgcccaccggagggcatgggaagatgccgagaaggactgccgccgccgctccggccacctgaccagcgtccactcaccggaggaacacagcttcattaatagctttgggcatgaaaacacgtggatcggcctgaacgacaggatcgtggagagagatttccagtggacggacaacaccgggctgcaatttgagaactggcgagagaaccagccggacaatttcttcgcgggtggcgaggactgtgtggtgatggtggcgcatgaaagcgggcgctggaacgatgtcccctgcaactacaacctaccctatgtctgcaagaagggcacagtgctctgtggtccccctccggcagtggagaatgcctcactcatcggtgcccgcaaggccaagtacaatgtccatgccactgtaaggtaccagtgcaatgaaggatttgcccagcaccatgtggccaccattcgatgccggagcaatggcaagtgggacaggccccaaattgtctgcaccaaacccagacgttcacatcggatgcggcgacaccaccaccaccaccaacaccaccaccagcatcaccaccacaaatcccgcaaggagcgcagaaaacacaagaaacacccaacggaggactgggagaaggacgaagggaatttctgctgaagaaccagaaaaaagaaagcacaacacctttcccatgcctcctctggagccttcgcctggggagacagaacccagagagaaacaagagagtccagaagtccctgaaccccaaaccacatcccccacacacataatcctttgtacaaagctcctcttttccctttttttacatacacaagatcctcttggcaggtggagccaggtgtctgaaaagttcattctcgtctggctgaactctgggagtgtgtcccagctgagggaagcacaagtagcaaagctcattggtctggtctcttgtttgccaggctgattgaagcaggccttgatgagggtgcatgagtgtatgtttgcattcacatgaaggaattgcttttcacaccagaaattcagacttagtcaatgttggctgaattcctaaatccaggaagaagcctggacgtagggtcattagctttgggaatagaaggctacacagaagcacactgtttttgaacttgacaacagctctccctttaccctggacttcagcccaagttccgtctttggtcttggtggataaacacacagtgtggagatcccacgtactgcattttagggatgtttttaggacaacctccctccatgccttcagagttaggagtgagaatgatcaaagcaatatgtaggtgatggagggagagtgtattgctaacccttccaggtctagtccagcgctgagatttggtggttctgcatgtgtgatgaatctctttcacacaaatagacgagaggatatttagggctagatgagcccagatttcttccccctccatctctcagggagacaaagaacctccttcctggaccaaggaggtgctgccaagttttctagcccagtgcacatacccagtccttaagcagacattggtagtgcccctgccctgggtcccactcctgccccaccccacccttgtccctggccattgcctggtggtctagaaacacttaaaacttgaagtagtgacacctacctgcggtcatattgtagagagatgctcagtgttaaaactgaaacacacaaacacacacacacacacatttttctcttgtagattttaattttttaagtgggaaagaactcaccttgccttcctcccccaaatgtgcaacctgtaaaaggtctctccacaccaggggccaggatccagttccctcatctctggcaggaaagatccacagcttttcctccatgtctgttactcactttcagcagtccgggtaaaatctgtggatcagggttaaaaaagcaccgtggagaatggccctcttcaggaaagaaaaataagcaaatgaatggtccacctaggggttcagtaaagaaagaaatgtgttaactgagcctgaatcccttctgggaagtaataatgaccattgacaactaagaagtagacaccatgctaaagacttacatacaatctccttgaatcttctcaatagcccattgacttagaaactgttactttcccattttacacacagtgaaactgaggctcagatataaaggaaaggtactggcttgaagtcacaaccacgacaggagtaaggatttggaataaggatttggtcctgttttctggaccaaatccttactctggctctgcttacactttctctccatcaccaaatccttactccaaatccagaagtcagagccaactcccatcttggttctgacccaaatcctgctctggactctggagaggagattgaaatataattgcaccctcatacacatttaggaaatggttaagaagtgtaaactgaacccttatccttgtcttcaatcttcctccctgtagacatctatcttattatggttattattcagaaaacccagggatacaggtttgtcttcttactttgataactcttcttagtttaaaataataataataacacatctttggtcatctatgtcacacaaaaattttcctttgtttgcggggggctggggatgcagtgttttttggggggtcttggtttatgctccctgcccttgagcccctcagccgtttgccctgcccccacctcggctccatggtgggagggggctctggtcttttctaaagtgggcggtttgtcttttgatctttcccttttggatgtgcgtgtgtgtctgcgtgtgccatgtgcgtggcacgcatatgagtgtgtgtgcgtgtgaacggctttgggtcctgctggttttgctgtgagctgcagtgttctgtgggtctgtggtatctgacactgtggacattaatgtacttcttggacattttaataaattttttaacagttcaa

SEQ ID NO: 22 - RNA, 12774 nt, Homo sapiens
  source 1..12774: mol_type = mRNA
acgtcagaggcaggaaccgactgtgctaaggctgttggctcaacacccagaggagacgggcagacccagggagagtgagagaagggggtcctctctgacccaaggaattaccactagtggagtgaagccacctgactttttgatcttattttggttgcctcctcattctccttccacccgtagccctgacagcttgggtttcatttctttcgtggagccttgtctcttcctcccagaataggaggaagggaagagaagggaaagaggagggctctctaggtgagcgcatcagctggctccagcctgagcaagcaagaattttcttcccaggaagctcctctcgctccccggccgcccacccccagcctgggtggctgtatcgttttaactgcatagagggcaggtctcttttggaattaggattaaagaaagtgcagtaaagagaaagcatcgaagacaccatcacaaaagattcccacaactccatgctgtgtgctgcaggctggtcctgaacccagatctctggctgagaggatgggggcagatggggaaacagtggttctgaagaacatgctcattggcatcaacctgatccttctgggctccatgatcaagccttcagagtgtcagctggaggtcaccacagaaagggtccagagacagtcagtggaggaggagggaggcattgccaactacaacacatccagcaaagagcagcctgtggtcttcaaccacgtgtacaacattaacgtgcccttggacaacctctgctcctcagggctagaggcctctgctgagcaggaggtgagtgcagaagacgagactctggcagagtacatgggccagacctcagaccacgagagccaggtcacctttacacacaggatcaacttccccaaaaaggcctgtccatgtgccagttcagcccaggtgctgcaggagctgctgagccggatcgagatgctggagagggaggtgtcggtgctgcgagaccagtgcaacgccaactgctgccaagaaagtgctgccacaggacaactggactatatccctcactgcagtggccacggcaactttagctttgagtcctgtggctgcatctgcaacgaaggctggtttggcaagaattgctcggagccctactgcccgctgggttgctccagccggggggtgtgtgtggatggccagtgcatctgtgacagcgagtacagcggggatgactgttccgaactccggtgcccaacagactgcagctcccgggggctctgcgtggacggggagtgtgtctgtgaagagccctacactggcgaggactgcagggaactgaggtgccctggggactgttcggggaaggggagatgtgccaacggtacctgtttatgcgaggagggctacgttggtgaggactgcggccagcggcagtgtctgaatgcctgcagtgggcgaggacaatgtgaggaggggctctgcgtctgtgaagagggctaccagggccctgactgctcagcagttgcccctccagaggacttgcgagtggctggtatcagcgacaggtccattgagctggaatgggacgggccgatggcagtgacggaatatgtgatctcttaccagccgacggccctggggggcctccagctccagcagcgggtgcctggagattggagtggtgtcaccatcacggagctggagccaggtctcacctacaacatcagcgtctacgctgtcattagcaacatcctcagccttcccatcactgccaaggtggccacccatctctccactcctcaagggctacaatttaagacgatcacagagaccaccgtggaggtgcagtgggagcccttctcattttccttcgatgggtgggaaatcagcttcattccaaagaacaatgaagggggagtgattgctcaggtccccagcgatgttacgtcctttaaccagacaggactaaagcctggggaggaatacattgtcaatgtggtggctctgaaagaacaggcccgcagcccccctacctcggccagcgtctccacagtcattgacggccccacgcagatcctggttcgcgatgtctcggacactgtggcttttgtggagtggattccccctcgagccaaagtcgatttcattcttttgaaatatggcctggtgggcggggaaggtgggaggaccaccttccggctgcagcctcccctgagccaatactcagtgcaggccctgcggcctggctcccgatacgaggtgtcagtcagtgccgtccgagggaccaacgagagcgattctgccaccactcagttcacaacagagatcgatgcccccaagaacttgcgagttggttctcgcacagcaaccagccttgacctcgagtgggataacagtgaagccgaagttcaggagtacaaggttgtgtacagcaccctggcgggtgagcaatatcatgaggtactggtccccaggggcattggtccaaccaccagggccaccctgacagatctggtacctggcactgagtatggagttggaatatctgccgtcatgaactcacagcaaagcgtgccagccaccatgaatgccaggactgaacttgacagtccccgagacctcatggtgacagcctcctcggagacctccatctccctcatctggaccaaggccagtggccccattgaccactaccgaattacctttaccccatcctctgggattgcctcagaagtcaccgtacccaaggacaggacctcatacacactaacagatctagagcctggggcagagtacatcatttccgtcactgctgagaggggtcggcagcagagcttggagtccactgtggatgctttcacaggcttccgtcccatctctcatctgcacttttctcatgtgacctcctccagtgtgaacatcacttggagtgatccatctcccccagcagacagactcattcttaactacagccccagggatgaggaggaagagatgatggaggtctccctggatgccaccaagaggcatgctgtcctgatgggcctgcaaccagccacagagtatattgtgaaccttgtggctgtccatggcacagtgacctctgagcccattgtgggctccatcaccacaggaattgatcccccaaaagacatcacaattagcaatgtgaccaaggactcagtgatggtctcctggagccctcctgttgcatctttcgattactaccgagtatcatatcgacccacccaagtgggacgactagacagctcagtggtgcccaacactgtgacagaattcaccatcaccagactgaacccagctaccgaatacgaaatcagcctcaacagcgtgcggggcagggaggaaagcgagcgcatctgtactcttgtgcacacagccatggacaaccctgtggatctgattgctaccaatatcactccaacagaagccctgctgcagtggaaggcaccagtgggtgaggtggagaactacgtcattgttcttacacactttgcagtcgctggagagaccatccttgttgacggagtcagtgaggaatttcggcttgttgacctgcttcctagcacccactatactgccaccatgtatgccaccaatggacctctcaccagtggcaccatcagcaccaacttttctactctcctggaccctccggcaaacctgacagccagtgaagtcaccagacaaagtgccctgatctcctggcagcctcccagggcagagattgaaaattatgtcttgacctacaaatccaccgatggaagccgcaaggagctgattgtggatgcagaagacacctggattcgactggagggcctgttggagaacacagactacacggtgctcctgcaggcagcacaggacaccacgtggagcagcatcacctccaccgctttcaccacaggaggccgggtgttccctcatccccaagactgtgcccagcatttgatgaatggagacactttgagtggggtttaccccatcttcctcaatggggagctgagccagaaattacaagtgtactgtgatatgaccaccgacgggggcggctggattgtattccagaggcggcagaatggccaaactgattttttccggaaatgggctgattaccgtgttggcttcgggaacgtggaggatgagttctggctggggctggacaatatacacaggatcacatcccagggccgctatgagctgcgcgtggacatgcgggatggccaagaggccgccttcgcctcctacgacaggttctctgtcgaggacagcagaaacctgtacaaactccgcataggaagctacaacggcactgcgggggactccctcagctatcatcaaggacgccctttctccacagaggatagagacaatgatgttgcagtgactaactgtgccatgtcgtacaagggagcatggtggtataagaactgccaccggaccaacctcaatgggaagtacggggagtccaggcacagtcagggcatcaactggtaccattggaaaggccatgagttctccatcccctttgtggaaatgaagatgcgcccctacaaccaccgtctcatggcagggagaaaacggcagtccttacagttctgagcagtgggcggctgcaagccaaccaatattttctgtcatttgtttgtattttataatatgaaacaaggggggagggtaatagcaatgtgttttgcaacatattaagagtatgtgaaggaagcagggatgtcgcaggaatccgctggctaacatctgctcttggtttctgctgccctggagcctgaccctcagtctccattctccctcctacccaggcctcctcaaccttcacctcctttcccaccaaggaggagaagtaggaagttttcttaaagggccaattcaaagccaagtcgtggggtgcagattgttatggtgacaggcacacacatttttctacccttcttctgagatgtcctctgccttccaggtatttgtgattttgtcacagcctgacatggccaggttctcacactggcccagagaaaagagcctcagcaagagagttttgccaacaattccccttaaaaggaaacagatcaactacaccgcatcccaacaacccaggttcttttccttccttccttccttcctcccttccttctttcctgccttccctccttccctcctttctttcttgacagtatctcactcttgctcagtctggagtgcagtggtgcaatctcagctcactacagcctcgacttcctgagcttaagtgatcctctcgtctcagcctcttgagtagctgggactacaggcatgagccaccatacctggctgatttttcttttattttttgtagagatgggggtctcactgtgttgtccaggctggtcacaaactcctggccttgaacaatcctcccacctcagcctcccaaaatgctgggattacaggctacagccactgcatccagccccaagtcctttataatgggccaaagattctcatcttctgacactcttattataccagccacatgtcctgttacctttcaagaacagtggctcttttctggccctgatggagtcttttgcctaatcccatcctgtcttagaccccagagcctcaaaagcccaaccagcattttggaatctagttcccaaggcattgcctgctcctggccctgggtggcacagtggctatgtgcctgtgagtcatcagccactgcccccaccctcaggtcagacagacagtcactttctgcaaaggtaggagggggagatggttgtagtttttagcacttttgggtcaagcattgaggaaaggactgaagaagtcaaaatcagggcactgccatagagattatagtttccatgggaaacacagatcttccctagccctgttttctccatttctggtcctatttgtcacagattctttttttctgaattgcaaaccgaaggcctcatatctatcttcacccaagccctccttagtgtaggtggcttaataagagtcaggagaagaaggtatatgcccagagagctatacccctgactgataatcctggaaagaggttgattttttttttcttttttggtcctatgggaaacaggaatgaccttcttaatgaggtgagatcatgagaagcctttccatcttcttttattattctgccttgcaaaggaaaaatgacaatagccaaaggaaattacatccagaaatccaaaggtgaataggtgataactgtgggcctacttcccattttatgagatgcactgtcttcactagcacaatttgatcttaagcccaagagagctagatgctctagaagccgtgacctttcaatttctcctccaaatccaatatcagcaaccatccccgcaacatctgcccagatccatgcccagttccctgagctgtcttctccctttcctctcccattcctcaaggcccagcaccattttctgtagtgcctatgtttgtctgaccagagcagtgtgtacctttgccaaccagttactggatgagccttagtgtcggcaattttctaatggaaatatcctcactcttaccaagctttgagaaccaagatgaaaagtcccttgggctgaccatcaggcctcggacctcatggggatgcagccagccaccattgaatggaggcgcttcccttgccgcaggcttctaggctttgttcactgcggcagtgtggatgtgacaggcataaccctgaaaaacctaggcaccacttgcatttctcagttaaccatgggcttcaacaaactgggaggaggaaaaaacagaaacagatcctttcaacaagcatgtaaattacagaagacttacctagagacggggcaacaatgaatagtaggaagcacattgcatgggaaacttgagggccgaggtacaggccaacagctgtcatcttagctacgacatgacatcggctgggttgtttcacctctctgggccagtttcttcatctgtcagatgagagattagactagaatgagttctaatttccctttcagttttgacggtcaatgatgctaatctgtgatctgaaaacctaagtagaaattggcatggtgggacagcacaattaggtaatgaaaattcactagactccctgtttaatgaggaaagaaggaaagaaggaaggaaggaaggaaggaaagaaggaaggggagggagggagggaaagaaaagagagagattgattcaggagtcataagtaaaacacacacacacacacacacacacacacgaaaatactgcagaaaagatcatgccgagtgagaggctgaactctttttgtccataaaaacagagaccaggtacctgggtggtgccagcacgcccacttcatcaaggaagttgaaatggaaaaagattatgtgttgtcaattcaggtgaaagtcaagaaacatcagtggaaacaaaaatcttccttttattgaaaccattctacactatggaacatttttataaatagtaccagcggcattgcatagctttattttgaagcaagaaagagacttcaaagatcagcttattcaaccctctataaaaggatttatagattccttttatttaatagggtgattttctcatgagttttcaccatgtttaaaagacactgtgccccactctcatcccaaggtctatggtcaacttgactcaatgctcataactttcctcgctgattcctgcttatcctgctcctccaagcacaactgcatttcccaaaggtagatccagtaagcaatactctcaaagcacccaccctatgcacaccagtttccctcaagggactcaaaaggaatcgaggtagacccttctcagaggctgtttaactacagacacatcaggatatgtactaatacccaaaaccaaaaccaccaccaatgactgcaatagattcactgtctagacccaaggtccaggcagtttcccttttttgcatttccccaacatataaagataatttccaacatatcagatgcacattcaccagggttatttctggaaggtgaaggggtcaggtgtagaagaggtggcaagaatgacaagtctgtggaaaactgttggccttgagaccacagtgaactggtggaaggggacttgctaagtgtggccacaaggtggcagtcacctcctgaaaccccgtgtgaaacgtcagcggggttgagaaaggccacgtctccaagtttccaggagagccaaaccaggactcaggaaaaccggccagctgatttgtatgggagattgatttgtaaaagaacaacaacaacatatggcagcaaggaaatgcaaagcaaacatctacaaatatttgaatccacctgtccctcacctaccaacttccccctctactactcataaagtggtaggactcctaatgccccgcttccttccacctgagaaccaaccagcaccctgatatttcaaagccagacctaaagaaccatggttgccaataacctcaatgccaaaccaaacaatcctaatcacatttttacttttcaaatccagtctcagagcagagctgtagattaagggacaaaaagtcattgtggaatcttttcaaatagctcttgtaaaagcatcagttgggaaagagtttatgagttcctctttgtcctctcctctggagaaaagacagaacatgcaagaacactagatacaccaagctatgggtgttgaaatagactagctggtgacctggatttgcaaataccctagggacatgaaaacattttttctttttctttttcttttttgtaacctccacccagaatttcaggaaaggtggggccacatgcccaggatatatggtattctactgcttgctttcttatcaccagaatactagtcaatctgatttcttagttccatgtcttttcttccaaggaggtagggcatggggttgggggacactttccgccttccttgcatctcactgtccactcttaaatttttattaagaaacctctgaaagattcaaaattcagttcagtttcaataatcccaaagaagttgggataatttgttttcacttgcttcaaacttgccacggctgcaggaggagtctcaggacctcagaggaatggggcagatgtgtgaagtctgggagtctgcctcccacccagatgccaggagctgcatactcagctctgcctcacttccctgatggtctgctttcatccaggttaccatgacagagagggtctatcctctaaagaaagctgagttacaggctggcccctggggttgttcctgtagcctctctttgaccacattcctgaagatgtttagctgaatgcctcctcaaacctttgcttcttccataacccaatcctacacatatgtgataaagctttcttcaacaatactgggtcatttggacactaccagtccagatgagaactcacagaccaaatgaaaactttcaaaagcaagcagggaaagtaactcctatatgctttaagatgatgtattggtgtagattcaggtccaatttgaaaaaatgtctctggttctttctaattgtatccactctggatttgtttatgccagttctacattcatcaagccttgagaaacaggtggaaagaagaaaaatataaaattaagaagaagctgatggatcattggagattcaaaacaagccttctaccggtggcaccacactgaaaggcagagacaaggcaggagcggccatgggagaggaagcagcattggcttctcacgttctggcctccagcctcccaccagagcttctgccattcacccacttaacttgaggagaaactaaggcaccaaggcctgtcactgcaccgaaaaccagaagcagaaggactttctttctttgctcaaccagttctgcctggatctgacttttgcgtcaccagtagtcacaagcctgatttgtcatctccagcattctgtttatttttttaaggtcaatctttgcctgaaacatgacttcattgcacctgcagcacttcattgtccaaatgtccttggcaaacctcacagtctttggacagtgttgcattttctccaaaggggaaatcctctctccttcctcaagcttaataaggctcttgcagtgcatccaaataacacagctcttgttcctcagacacactcagccccctctgataacttcataaaacagctccttattgtctgggccatgaagccaggctaatgctaaagttccagttcaaaaacatttaactctattatccttatgaggccagctctcctgggtactggcactcactcactggcaccatggattcaggtcaggtggtgcaaacatcttccctgagaagcaagccatacacccaacaggtctagaacacctaggtcacaagaaagggtttcagtccagatgcactgtcagtaagtgaacacaaggacaagaatgcatgaactgttattttcttcccatcactggtttagcagcccctagggagaccctgtgttgctctttaatattctgacatggagcactaatatgctcaacactgcctccaacaggcaaatgttggaaatacactttcagagctggatgggactcaagatcatctaatataatcctctcattttatttcctctcaaggaaaccaagggcaagaaagtttaggcaatttgctcacattcacacaggcaattagtaacagagctgagttcgggtctggtataggctttttaatttgattctccacaaatgaacactaagataaatagcatttattgcatacccactatttaccagatactgtgcaaagggctttacatatattactttactaaatcctcaccataaactttaagtcaggtttaaccattttatagaagagggaagggagggccaggaaggttaagtaacatgctcaaagtcacacagctactaaatagagccataatctgaacctttgtctaattttgaagcccttgatttttccacttcatgaggctgctctttatatttatttagctgagtgaccaccatcttggattttactaatattcttacaagggggctttgatgccacttcttctgacttttaagaaaaggaaattcttgcattatttttgccatgtactccaggggaccctcaatctcatcatcatggggaataagaaacattctgacatctctttccttttctttctttttaaatttggtagacgagggagagggtccccaacagaatgagagggggaagataagaaccagaacataaacccaataccccccaccccactcttttgggaacatcccaacattagaagagtgggatatccagtcatttttttcaagctggagcaggcagtgtagccaggattgaggaaggagaagccacagaataaacatggcacatgttctcagagcatcaattttactccagaatttgggaagaaaggattatattattagtgtattcaaaccagtatggatagcacaaatgcagatatcctataaattttgaggctaactccagcaggttgtagactgatttaggctacctccttccagactcccaggggtacaaacaccttcacagtcccctgcgggagtacatcagtggaaatatttggggaatgaggctagaaggcaggagatacattcagtttacttatccccctggtttcttttccccactccttacctgccccacatccctagtgggtgggaaggcatcagaagacagttggctacaatatcagctttctctgctactttggggaatttagaaggaaatcagtacttggcaacctggtatttttagctgccaggccaaaactccagacataaaccccttccctttaattgaatgatgtttatatataaaggaatagatctccccaagaaagaaccccagaacactggttccattccagtgtgggggctgcttgggcagcagtgacacctgtgcattagatgcaatagacacttcaattcctgcaaaaatgtcggccaactaagtgctcctttaccaactgcggaggcccagactcacaccatggtcccacagactcaaggtttagaatctatgtgagtctattttggttaacacacaggctcctccttccaatcattcacacacacacacacacacacacacacacacacatgcacacacacacatgcatgcacacatacacaggtgcacatgcacaaacacacacatctaatgattccaacctcgaatactccccaggcccatggggatgcccaagtaccctaagcaaatcactgtgacactgagggggtcattaactggggctccgagcagctgaaacccaccctggtggtctctagattgttgttctgagaaatcataaacaccaaatcctccaatccctttgaatttgagtttgtcattttaacctttaacagccttttcaggtaaaaataacaaagaccaatttattgactttgtagctacaatgagcttgaaaaagcgcttacttaaaagaaagactggaaggaagaaaaaaaaaagaaaatggagtacatactcctcagacgatgcacactttaattagctaaaataaaaataaaaatgtacagaggaagggagaggaaaaaccaacttctctgatcctttcatggcactgtagctcaaagactggaatctctgcttgttaaccattgttaactagtatttttagtcctggttgcttttagcctctgtatagttccttttttaaacacattttctatacgttaataaaagatcctgaaagaaa

SEQ ID NO: 23-29 - skipped (7 entries)

SEQ ID NO: 30 - RNA, 21 nt, synthetic construct
  source 1..21: mol_type = other RNA
ntaatgacagcgtagacgntg

SEQ ID NO: 31 - RNA, 21 nt, synthetic construct
  source 1..21: mol_type = other RNA
ntgctaatgacagcgtagncg

SEQ ID NO: 32 - RNA, 21 nt, synthetic construct
  source 1..21: mol_type = other RNA
ncagcgtagacgctgatgntg

SEQ ID NO: 33 - RNA, 21 nt, synthetic construct
  source 1..21: mol_type = other RNA
ntggtccggtggcagttcnta

SEQ ID NO: 34 - RNA, 21 nt, synthetic construct
  source 1..21: mol_type = other RNA
nacggtggtctctgtgatngt

SEQ ID NO: 35 - RNA, 21 nt, synthetic construct
  source 1..21: mol_type = other RNA
nacagcgtagacgctgatntt

SEQ ID NO: 36 - RNA, 21 nt, synthetic construct
  source 1..21: mol_type = other RNA
nttgctaatgacagcgtanac

SEQ ID NO: 37 - RNA, 21 nt, synthetic construct
  source 1..21: mol_type = other RNA
nctaatgacagcgtagacnct

SEQ ID NO: 38 - RNA, 21 nt, synthetic construct
  source 1..21: mol_type = other RNA
ngttgctaatgacagcgtnga

SEQ ID NO: 39 - RNA, 21 nt, synthetic construct
  source 1..21: mol_type = other RNA
nccaatggtaccagttgangc

SEQ ID NO: 40 - RNA, 21 nt, synthetic construct
  source 1..21: mol_type = other RNA
nccggtggcagttcttatncc

SEQ ID NO: 41 - RNA, 21 nt, synthetic construct
  source 1..21: mol_type = other RNA
ncggtggcagttcttatanca

SEQ ID NO: 42 - RNA, 21 nt, synthetic construct
  source 1..21: mol_type = other RNA
nagcgtagacgctgatgtngt

SEQ ID NO: 43 - RNA, 21 nt, synthetic construct
  source 1..21: mol_type = other RNA
ncaatggtaccagttgatncc

SEQ ID NO: 44 - RNA, 19 nt, synthetic construct
  source 1..19: mol_type = other RNA
ncgtctacgctgtcattan

SEQ ID NO: 45 - RNA, 19 nt, synthetic construct
  source 1..19: mol_type = other RNA
nctacgctgtcattagcan

SEQ ID NO: 46 - RNA, 19 nt, synthetic construct
  source 1..19: mol_type = other RNA
ncatcagcgtctacgctgn

SEQ ID NO: 47 - RNA, 19 nt, synthetic construct
  source 1..19: mol_type = other RNA
ngaactgccaccggaccan

SEQ ID NO: 48 - RNA, 19 nt, synthetic construct
  source 1..19: mol_type = other RNA
natcacagagaccaccgtn

SEQ ID NO: 49 - RNA, 19 nt, synthetic construct
  source 1..19: mol_type = other RNA
natcagcgtctacgctgtn

SEQ ID NO: 50 - RNA, 19 nt, synthetic construct
  source 1..19: mol_type = other RNA
ntacgctgtcattagcaan

SEQ ID NO: 51 - RNA, 19 nt, synthetic construct
  source 1..19: mol_type = other RNA
ngtctacgctgtcattagn

SEQ ID NO: 52 - RNA, 19 nt, synthetic construct
  source 1..19: mol_type = other RNA
nacgctgtcattagcaacn

SEQ ID NO: 53 - RNA, 19 nt, synthetic construct
  source 1..19: mol_type = other RNA
ntcaactggtaccattggn

SEQ ID NO: 54 - RNA, 19 nt, synthetic construct
  source 1..19: mol_type = other RNA
nataagaactgccaccggn

SEQ ID NO: 55 - RNA, 19 nt, synthetic construct
  source 1..19: mol_type = other RNA
ntataagaactgccaccgn

SEQ ID NO: 56 - RNA, 19 nt, synthetic construct
  source 1..19: mol_type = other RNA
nacatcagcgtctacgctn

SEQ ID NO: 57 - RNA, 19 nt, synthetic construct
  source 1..19: mol_type = other RNA
natcaactggtaccattgn

SEQ ID NO: 58 - RNA, 21 nt, synthetic construct
  source 1..21: mol_type = other RNA
ctaatgacagcgtagacgctg

SEQ ID NO: 59 - RNA, 21 nt, synthetic construct
  source 1..21: mol_type = other RNA
ttgctaatgacagcgtagacg

SEQ ID NO: 60 - RNA, 21 nt, synthetic construct
  source 1..21: mol_type = other RNA
acagcgtagacgctgatgttg

SEQ ID NO: 61 - RNA, 21 nt, synthetic construct
  source 1..21: mol_type = other RNA
ttggtccggtggcagttctta

SEQ ID NO: 62 - RNA, 21 nt, synthetic construct
  source 1..21: mol_type = other RNA
cacggtggtctctgtgatcgt

SEQ ID NO: 63 - RNA, 21 nt, synthetic construct
  source 1..21: mol_type = other RNA
gacagcgtagacgctgatgtt

SEQ ID NO: 64 - RNA, 21 nt, synthetic construct
  source 1..21: mol_type = other RNA
gttgctaatgacagcgtagac

SEQ ID NO: 65 - RNA, 21 nt, synthetic construct
  source 1..21: mol_type = other RNA
gctaatgacagcgtagacgct

SEQ ID NO: 66 - RNA, 21 nt, synthetic construct
  source 1..21: mol_type = other RNA
tgttgctaatgacagcgtaga

SEQ ID NO: 67 - RNA, 21 nt, synthetic construct
  source 1..21: mol_type = other RNA
tccaatggtaccagttgatgc

SEQ ID NO: 68 - RNA, 21 nt, synthetic construct
  source 1..21: mol_type = other RNA
tccggtggcagttcttatacc

SEQ ID NO: 69 - RNA, 21 nt, synthetic construct
  source 1..21: mol_type = other RNA
ccggtggcagttcttatacca

SEQ ID NO: 70 - RNA, 21 nt, synthetic construct
  source 1..21: mol_type = other RNA
cagcgtagacgctgatgttgt

SEQ ID NO: 71 - RNA, 21 nt, synthetic construct
  source 1..21: mol_type = other RNA
ccaatggtaccagttgatgcc

SEQ ID NO: 72 - RNA, 19 nt, synthetic construct
  source 1..19: mol_type = other RNA
gcgtctacgctgtcattag

SEQ ID NO: 73 - RNA, 19 nt, synthetic construct
  source 1..19: mol_type = other RNA
tctacgctgtcattagcaa

SEQ ID NO: 74 - RNA, 19 nt, synthetic construct
  source 1..19: mol_type = other RNA
acatcagcgtctacgctgt

SEQ ID NO: 75 - RNA, 19 nt, synthetic construct
  source 1..19: mol_type = other RNA
agaactgccaccggaccaa

SEQ ID NO: 76 - RNA, 19 nt, synthetic construct
  source 1..19: mol_type = other RNA
gatcacagagaccaccgtg

SEQ ID NO: 77 - RNA, 19 nt, synthetic construct
  source 1..19: mol_type = other RNA
catcagcgtctacgctgtc

SEQ ID NO: 78 - RNA, 19 nt, synthetic construct
  source 1..19: mol_type = other RNA
ctacgctgtcattagcaac

SEQ ID NO: 79 - RNA, 19 nt, synthetic construct
  source 1..19: mol_type = other RNA
cgtctacgctgtcattagc

SEQ ID NO: 80 - RNA, 19 nt, synthetic construct
  source 1..19: mol_type = other RNA
tacgctgtcattagcaaca

SEQ ID NO: 81 - RNA, 19 nt, synthetic construct
  source 1..19: mol_type = other RNA
atcaactggtaccattgga

SEQ ID NO: 82 - RNA, 19 nt, synthetic construct
  source 1..19: mol_type = other RNA
tataagaactgccaccgga

SEQ ID NO: 83 - RNA, 19 nt, synthetic construct
  source 1..19: mol_type = other RNA
gtataagaactgccaccgg

SEQ ID NO: 84 - RNA, 19 nt, synthetic construct
  source 1..19: mol_type = other RNA
aacatcagcgtctacgctg

SEQ ID NO: 85 - RNA, 19 nt, synthetic construct
  source 1..19: mol_type = other RNA
catcaactggtaccattgg

SEQ ID NO: 86 - RNA, 21 nt, synthetic construct
  source 1..21: mol_type = other RNA
aacagtggcatggacattcta

SEQ ID NO: 87 - RNA, 21 nt, synthetic construct
  source 1..21: mol_type = other RNA
aacagtggcatggacattgta

SEQ ID NO: 88 - RNA, 21 nt, synthetic construct
  source 1..21: mol_type = other RNA
tgagctcggaagcagtaggcg

SEQ ID NO: 89 - RNA, 21 nt, synthetic construct
  source 1..21: mol_type = other RNA
agagctcggaagcagtagccg

SEQ ID NO: 90 - RNA, 21 nt, synthetic construct
  source 1..21: mol_type = other RNA
agagctcggaagcagtaggcg

SEQ ID NO: 91 - RNA, 21 nt, synthetic construct
  source 1..21: mol_type = other RNA
agggcctcagcgaaggtccgt

SEQ ID NO: 92 - RNA, 21 nt, synthetic construct
  source 1..21: mol_type = other RNA
tgggcctcagcgaaggtcggt

SEQ ID NO: 93 - RNA, 21 nt, synthetic construct
  source 1..21: mol_type = other RNA
agggcctcagcgaaggtcggt

SEQ ID NO: 94 - RNA, 21 nt, synthetic construct
  source 1..21: mol_type = other RNA
tgggcctcagcgaaggtcagt

SEQ ID NO: 95 - RNA, 21 nt, synthetic construct
  source 1..21: mol_type = other RNA
agggcctcagcgaaggtcagt

SEQ ID NO: 96 - RNA, 19 nt, synthetic construct
  source 1..19: mol_type = other RNA
gaatgtccatgccactgtt

SEQ ID NO: 97 - RNA, 19 nt, synthetic construct
  source 1..19: mol_type = other RNA
caatgtccatgccactgtt

SEQ ID NO: 98 - RNA, 19 nt, synthetic construct
  source 1..19: mol_type = other RNA
cctactgcttccgagctca

SEQ ID NO: 99 - RNA, 19 nt, synthetic construct
  source 1..19: mol_type = other RNA
gctactgcttccgagctct

SEQ ID NO: 100 - RNA, 19 nt, synthetic construct
  source 1..19: mol_type = other RNA
cctactgcttccgagctct

SEQ ID NO: 101 - RNA, 19 nt, synthetic construct
  source 1..19: mol_type = other RNA
ggaccttcgctgaggccct

SEQ ID NO: 102 - RNA, 19 nt, synthetic construct
  source 1..19: mol_type = other RNA
cgaccttcgctgaggccca

SEQ ID NO: 103 - RNA, 19 nt, synthetic construct
  source 1..19: mol_type = other RNA
cgaccttcgctgaggccct

SEQ ID NO: 104 - RNA, 19 nt, synthetic construct
  source 1..19: mol_type = other RNA
tgaccttcgctgaggccca

SEQ ID NO: 105 - RNA, 19 nt, synthetic construct
  source 1..19: mol_type = other RNA
tgaccttcgctgaggccct